SEQUENCE LISTING (16 sequences)

SEQ ID NO: 1 - protein, 8 aa, Adeno-associated virus
FHCHFSPR

SEQ ID NO: 2 - protein, 9 aa, Adeno-associated virus
NWLPGPCYR

SEQ ID NO: 3 - protein, 11 aa, Adeno-associated virus
QLDSGDNPYLK

SEQ ID NO: 4 - protein, 12 aa, Adeno-associated virus
TNTPSGTTTQSR

SEQ ID NO: 5 - protein, 12 aa, Adeno-associated virus
LQFSQAGASDIR

SEQ ID NO: 6 - protein, 11 aa, Adeno-associated virus
YNHADAEFQER

SEQ ID NO: 7 - protein, 13 aa, Adeno-associated virus
LKPGPPPPKPAER

SEQ ID NO: 8 - protein, 13 aa, Adeno-associated virus
LKEDTSFGGNLGR

SEQ ID NO: 9 - protein, 14 aa, Adeno-associated virus
RVLEPLGLVEEPVK

SEQ ID NO: 10 - protein, 15 aa, Adeno-associated virus
KRVLEPLGLVEEPVK

SEQ ID NO: 11 - protein, 16 aa, Adeno-associated virus
LQFSQAGASDIRDQSR

SEQ ID NO: 12 - protein, 19 aa, Adeno-associated virus
LKEDTSFGGNLGRAVFQAK

SEQ ID NO: 13 - protein, 19 aa, Adeno-associated virus
TTNPVATEQYGSVSTNLQR

SEQ ID NO: 14 - protein, 20 aa, Adeno-associated virus
YHLNGRDSLVNPGPAMASHK

SEQ ID NO: 15 - protein, 26 aa, Adeno-associated virus
YLGPFNGLDKGEPVNEADAAALEHDK

SEQ ID NO: 16 - protein, 31 aa, Adeno-associated virus
DSLVNPGPAMASHKDDEEKFFPQSGVLIFGK

(19) United States
(12) Patent Application Publication
(10) Pub. No.: US 2009/0017062 A1
(43) Pub. Date: Jan. 15, 2009

(54) Methods and compounds to alter virus infection

(75) Inventors: John F. Engelhardt, Iowa City, IA (US); Liang Zhang, Toronto (CA)

Correspondence Address: SCHWEGMAN, LUNDBERG & WOESSNER, P.A., P.O. BOX 2938, MINNEAPOLIS, MN, 55402, US

(73) Assignee: Iowa Research Foundation Iowa Centers for Enterpri, Iowa City, IA (US)

(21) Appl. No.: 11/796,605

(22) Filed: Apr. 27, 2007

Related U.S. Application Data
(60) Provisional application No. 60/796,109, filed on Apr. 28, 2006.
(60) Provisional application No. 60/857,349, filed on Nov. 7, 2006.

(51) Int. Cl. A61K 39/23; C12Q 1/70; A61P 31/20

(52) U.S. Cl. 424/204.1; 435/5

(57) ABSTRACT

The invention provides a method to identify an agent that alters parvovirus transduction of mammalian cells. Also provided is a method to enhance transgene expression in a mammalian cell, as well as a method to identify an agent that alters NADPH oxidase activity in parvovirus transduced mammalian cells.

### CROSS-REFERENCE TO RELATED APPLICATIONS

This application claims the benefit of the filing date of U.S. application Ser. No. 60/796,109, filed Apr. 28, 2006 and of U.S. application Ser. No. 60/857,349, filed Nov. 7, 2006, the disclosures of which are incorporated by reference herein.

### STATEMENT OF GOVERNMENT RIGHTS

The invention was made with a grant from the Government of the United States of America (grant HL58340 from the National Institutes of Health). The Government has certain rights in the invention.

### BACKGROUND

Reactive oxygen species (ROS) play essential roles in a variety of cell signaling processes by modulating protein phosphatases and thiol-regulated protein/protein interactions (Lambeth, 2004; Rhee et al., 2000). In phagocytes, pathogen-induced activation of the phagocytic NADPH oxidase (Nox2^{gp91phox}) complex leads to high levels of ROS in phagosomes that assist in the destruction of phagocytosed pathogens. Moreover, in a broad range of other cell types, ROS play important roles in mediating cellular signaling in response to a variety of ligands, such as platelet-derived growth factor (PDGF), tumor necrosis factor alpha (TNF-α), insulin, interleukin beta (IL-1β), and the like (Lambeth, 2004; Rhee et al., 2000). The mechanisms by which ROS facilitate cellular signaling involve reversible modification of thiol groups on the active site of proteins, among which a well studied example is protein tyrosine phosphatases (PTPs) (Rhee et al., 2000). Depending on the number of electrons transferred, redox modification of thiol groups can results in various products including disulfide bonds, sulfenic acid, sulfinic acid, sulfonic acid in addition to others (Paget et al., 2003).

Due to their highly reactive properties, cells compartmentalize ROS to restrict their sites of action to specific locations involved in signaling. For example, studies have implicated mitochondrial superoxide as a source of H₂O₂ responsible for the oxidative inactivation of JNK phosphatases important in TNF-mediated apoptosis (Kamata et al., 2005). Similarly, peroxiredoxin II (Prx II) has been shown to act as a negative regulator of PDGF signaling by controlling the activity of PTPs important in PDGF receptor inactivation (Choi et al., 2005). More recently, studies have also demonstrated that receptor-mediated endocytosis of ligand bound IL-1R1 stimulates Nox2-mediated endosomal ROS production and spatially restricts redox activation of the receptor complex (Li et al., 2006a; Li et al., 2006b).

In addition to the well established importance of ROS in cell signaling, increasing evidence suggests that ROS also play critical roles in the pathogenesis of many types of viral infections (McFadden, 1998; Schwarz, 1996; Shisler et al., 1998). In this context, many viruses are known to induce ROS generation during infection and as such also lead to the induction of genes responsible for clearing cellular ROS. Adenovirus and tumorigenic poxviruses can induce a cellular redox imbalance, which these viruses depend on to replicate (Rannan et al., 2004; Teoh et al., 2005). For example HIV, influenza virus, and hepatitis viruses are known to induce oxidative stress and antioxidant treatments have been reported to ameliorate the morbidity caused by these viruses (Cai et al., 2003; Loguercio et al., 2003; Nakamura et al., 2002; Oda et al., 1989; Newman et al., 1994). In an in vivo study of influenza A infection (Buffinton et al., 1992), the airway microenvironment of infected animals displayed signs of oxidative stress including increased superoxide generation and H₂O₂ formation, as well as decreased ascorbate levels. However, the antioxidant capacity of the infected lung was not impaired as compared with uninfected animals, suggesting a primary effect of influenza A on the generation of ROS. Antioxidant therapy against influenza A using conjugated SOD had proven to be effective, but only if the administration was within a specific period (Oda et al., 1989). In the case of HIV, it is generally thought that the oxidative stress facilitates its replication, and the mechanism involves redox-activated NF-κB, which could enhance viral gene expression (Baruchel et al., 1992; Pollard et al., 1994; Schreck et al., 1992; Schwarz, 1996). Studies using in vitro models have indicated the efficacy of some antioxidants in ameliorating morbidity from HIV infection (Droge et al., 1992; Mihm et al., 1991; Newman et al., 1994).

In contrast, the molluscum contagiosum virus (MCV) genome encodes for a glutathione peroxidase (Gpx)-like protein that helps to prevent oxidative stress-induced apoptosis, which is a defensive mechanism cells adapt to limit viral infection (McFadden, 1998; Shisler et al., 1998). Despite the fact that numerous viruses are known to induce cellular ROS following infection, the mechanisms by which changes in the cellular redox state either facilitate or inhibit viral infection/replication remain poorly understood.

### SUMMARY OF THE INVENTION

The invention provides methods and compounds to alter virus transduction by viruses that have redox sensitive intracellular pathways, and methods to modify viruses to alter their redox sensitivity. In one embodiment, methods to enhance virus transduction of mammalian cells are provided. In one embodiment, the invention provides a method to enhance the transduction of recombinant parvovirus, e.g., recombinant adeno-associated virus (rAAV), using a compound that in an effective amount enhances ROS production, e.g., by enhancing endosomal NADPH oxidase activity, thereby enhancing gene transfer by those viruses. In another embodiment, methods to inhibit virus transduction of mammalian cells are provided. In one embodiment, the invention provides a method to inhibit parvovirus transduction using a compound that in an effective amount inhibits ROS production, for instance, by inhibiting endosomal NADPH oxidase activity. Further provided are methods to identify agents that enhance or inhibit redox sensitive intracellular virus processing pathways.

As described hereinbelow, adeno-associated virus type 2 (AAV2) has evolved to both stimulate endosomal ROS production during its infection and utilize the resultant hydrogen peroxide to facilitate endosomal processing of the virion. Infection of HeLa cells, IB3 cells, or primary mouse fibroblasts with rAAV2 stimulated endosomal NADPH-dependent superoxide production 3- to 4-fold. Removal of hydrogen peroxide from within the endosomal compartment by catalase loading significantly decreased transduction by rAAV2 about 80-fold. Given that Rac1 is important for rAAV2 transduction and is an activator of two NADPH oxidases (Nox1 and Nox2), Nox1 or Nox2 knockout (KO) and littermate wild type primary dermal fibroblasts were infected with AAV2. Results from these experiments demonstrated that Nox2^{-/-} fibroblasts failed to induce endosomal ROS following rAAV2 infection and had an 18-fold lower level of transduction as compared to wild type littermate fibroblasts. In contrast, no differences in rAAV2-induced endosomal ROS or transduction were observed in Nox1 KO and wild type littermate fibroblasts. These results suggested that AAV2 infection induces Nox2 to produce ROS in the endosomal compartment and that endosomal exposure of virus to H₂O₂ is important for productive intracellular processing of the virus.

As also described herein, a subclass of parvoviruses (e.g., AAV2) stimulates endosomal Nox2 during early stages of infection and utilizes the resultant H₂O₂ to promote sulfonic acid oxidation of Cys289 in capsid VPs. This redox event led to the partial unfolding of the AAV2 virion and activation of capsid VP1 phospholipase A₂ (PLA₂) activity required for endosomal escape of virions.

The invention thus provides a method to identify an agent that alters virus transduction of mammalian cells. The method includes contacting mammalian cells, one or more agents and virus suspected of having a redox sensitive intracellular pathway, and identifying one or more of the agents that alter endosomal NADPH oxidase activity relative to corresponding mammalian cells contacted with virus but not the one or more agents. Agents that inactivate the Nox complex that generates ROS in the endosomal compartment may be useful as anti-virals while agents that enhance ROS production through Nox may be useful to augment infection and so useful with gene therapy vectors or viral vaccines, i.e., to enhance their efficacy.

Accordingly, also provided are methods to enhance virus infection of mammalian cells, which include contacting mammalian cells with redox sensitive virus and an agent selected to enhance NADPH oxidase activity. Further provided are methods to inhibit virus infection of mammalian cells, which include contacting mammalian cells with redox sensitive virus and an agent selected to inhibit NADPH oxidase activity, e.g., apocynin or other compounds that target the multi-subunit Nox complex. In one embodiment, the virus is a pathogenic virus such as a pathogenic parvovirus, e.g., B19. In one embodiment, the agent is not a proteosome inhibitor or modulator.

As AAV2 enters into Rac1 containing endosomes, other viruses that show redox-dependent transduction or that utilize the Nox complex for transduction may have Rac1 dependent transduction pathways (since Rac1 is a co-activator of Nox). Hence, the findings that demonstrate that Rac1 co-localizes to the same endosome as AAV2 allows for the identification of new receptors responsible for entry of the virus using proteomic approaches of isolated HA-Rac1 tagged endosomes.

Thus, further provided are methods in which molecules in Rac containing endosomes from virally infected cells are identified. In one embodiment, Rac is labeled with a tag so that Rac containing endosomes may be identified and isolated. Once isolated, the proteomes of Rac containing endosomes with virus are compared to the proteomes of Rac containing endosomes from controls. Molecules that are present in the virus containing endosomes are candidates for receptors or co-receptors.

As also described herein, ROS-mediated endosomal processing of rAAV2 might involve redox-mediated changes to cysteine or other redox sensitive residues on capsids. Structural changes to purified virions exposed to H₂O₂ were mapped using MALDI TOFF MS. Results from these experiments suggest that nM quantities of H₂O₂ can enhance trypsin sensitivity of intact capsids. Thus, ROS may help to unfold the capsid while in the endosome and aid in activating certain biological function(s) of the virus. Treatment of intact rAAV2 virions with nM quantities of H₂O₂ also stimulated phospholipase A₂ activity resident in the viral capsids. These results suggest that AAV2 has evolved to both induce and utilize Nox2-derived ROS productively to process its virion during infection.

As modulation of parvovirus capsids is redox-sensitive, the viral capsid may be a target for improving parvovirus vectors, and redox modulation of capsid proteins in other types of viruses that have protein capsids may likewise improve viral vectors. Redox-modulation of a capsid with PLA₂ activity may involve the creation of new disulfide bonds through oxidation, and/or covalent modification of the capsid, e.g., modification of capsid residues including cysteines (sulfinic acid, sulfonic acid, sulfenic acid, and the like). Once cysteines or other redox modulatable amino acids, e.g., histidine, methionine, and the like, are identified, then amino acid substitutions, or other covalent modifications, may be engineered into redox-regulated portions of the capsid, which may improve infectivity in cells that fail to activate Nox following infection and/or improve virus production. Alternatively, identification of redox-modulated components in pathogenic parvovirus virions, e.g., in the capsids of pathogenic parvoviruses, may be useful to identify antiviral drugs with redox chemistries that inactivate virions.

Thus, the invention provides a method to identify viral capsid modifications that enhance virus transduction of mammalian cells. The method includes contacting mammalian cells and a virus having a modified viral capsid, wherein at least one modification is an alteration in the number or position (i.e., location) of redox-sensitive residues in the capsid or a post translational alteration that alters redox sensitivity of the capsid (e.g., abundance or placement of cysteines, methionines, lysines, histidines and other redox modifiable amino acids and disulfide bonds), and identifying whether the transduction of the mammalian cells by the modified virus is altered relative to transduction of corresponding mammalian cells by a corresponding unmodified virus. In another embodiment, mammalian cells are contacted with a library of viruses with capsid alterations and viruses with altered redox sensitivities, e.g., reduced sensitivity to redox stress, identified and characterized. Accordingly, the present invention provides for improved vector-design strategies for gene therapy to circumvent cellular barriers to viral transduction.

### BRIEF DESCRIPTION OF THE FIGURES

FIG. 1. Catalase loading does not affect AAV2 uptake. A) HeLa cells were treated with medium containing 1 mg/mL bovine catalase for 20 minutes prior to vesicular isolation. The vesicular fractions were then incubated with PBS (lane 1), pronase (lane 2), or pronase plus 0.5% Triton X-100 (lane 3) at 37° C. for 30 minutes. The samples were then resolved by SDS-PAGE and assayed by Western blot with anti-catalase antibody. B) HeLa cells were preincubated with AV2Luc (5×10³ particles/cell) for 1 hour at 4° C. in the absence or presence of 1 mg/mL catalase. Following washing, the infection was chased at 37° C. for indicated periods in control medium or medium containing 1 mg/mL catalase. Cells were then homogenized and the viral genome in the PNS quantified using Taqman PCR.

FIG. 2. AAV2 transduction is dependent on endosomal H₂O₂ and viral infection stimulates NADPH-dependent superoxide production in the endosomal compartment. A) HeLa or IB3 cells were pretreated with or without 1 mg/mL catalase 20 minutes before infection with AV2Luc (10³ particles/cell) in the absence or presence of proteasome inhibitors (40 μM LLnL and 5 μM doxorubincin). B) HeLa cells were preincubated with AV2Luc (5×10³ particles/cell) for 1 hour at 4° C. prior to removal of virus, shifting cells to 37° C., and chasing with catalase-containing medium (1 mg/mL) at various times post-infection. C) and D) HeLa and IB3 cells were treated with control medium, medium containing biotin-transferin (10 μg/ml), or AV2Luc (10³ particles/cell) for 20 minutes. Cells were then homogenized and the PNS loaded onto iodixanol-gradients for endosomal fractionation. Nox activity in each fraction was then determined. The Western blot at the bottom of C) depicts Rab5 (an early endosomal marker) distribution in the corresponding fractions. E) HeLa cells were treated combinations of SOD (1 mg/mL), catalase (1 mg/mL) and/or proteasome inhibitors (PI) [40 μM LLnL and 5 μM doxorubincin] prior to infection of AV2Luc (10³ particles/cell). In A) and E), catalase and/or SOD were continuously present during AAV2 infection. Relative luciferase activity was measured for each group 24 hours post-viral infection. Values represent mean±s.e.m. (n=4). Significant differences were analyzed using the Student t test for the marked comparisons.

FIG. 3. AAV2 co-localizes with Rac1-positive endosomes. HeLa cells were transfected with pEGFP-Rac1 for 24 hours prior to (A) no AAV2 infection, or (B-D) the binding of Alexa546-labeled AAV2 at 10⁴ particles/cell for 1 hour at 4° C. Virus was then removed by washing and cells were shifted to 37° C. for (B) 2 minutes, (C) 10 minutes, or (D) 30 minutes prior to fixation and analysis by confocal microscopy. Nuclei were stained with DAPI. b₁ and d₁ are magnification of boxed regions in panel B and D. Black and white panels to the right of color images are the corresponding green (EGFP-Rac1) or red (Alexa546-labeled AAV2) single channel images. Arrowheads depict several endosomes with colocalized Rac1 and AAV2. (E) The degree of AAV2-Rac1 colocalization in HeLa cells at different time points post-infection was determined using NIH ImageJ as described in the methods. Values represent mean+/−s.e.m. (n=10 cells at each time point).

FIG. 4. Nox2 is the primary source of endosomal ROS induced by AAV2 infection and is required for efficient transduction. a, Nox1 and Nox2 wild type (WT) and knockout (KO) PMDFs were infected with AV2Luc at an MOI of 10³ particles/cell in the presence or absence or catalase (1 mg/ml) and/or proteasome inhibitors (PI) [40 μM LLnL and 5 μM doxorubincin] added to the media as indicated. At 24 hours post-infection, relative luciferase activity was measured. Values represent mean±s.e.m. (n=4). b, Uptake of viral genomes in Nox2 KO and WT PMDFs was assessed following a 1 h 4° C. binding of 5×10³ particles/cell, washing of cells, and then the indicated chase period at 37° C. At the end of the chase period, cells were harvested and viral genomes in the PNS were quantified using Taqman PCR. c, NADPH-dependent superoxide production in the endosomal fraction of AV2Luc infected Nox2 WT and KO PMDFs. Vesicular fractions were isolated at 20 minutes post-infection with 10³ particles/cell. d, Total Nox activity in the endosomal fractions (fraction 2-4) are plotted, values represent the mean±s.e.m. (n=3). e, In vivo infection of Nox2 KO and WT mice lungs with 1×10¹¹ particles of AV2Luc in 5 μM Doxil (40 μl volume) using nasal aspiration. At 2 weeks post-infection, the relative luciferase activity in lung homogenates were assayed. Values represent mean±s.e.m. (n=4 independent animals for each time point). f, HeLa cells were infected with AV2Luc at an MOI of 1 particles/cell in the presence or absence of DPI (10 μM) and/or proteasome inhibitors (PI) in the media as indicated. 16 hours post-infection relative luciferase activity was measured. Values represent mean+/−s.e.m. (n=3). g. HeLa cells were infected with AV2Luc at an MOI of 103 particles/cell in control medium (vehicle) or in medium containing antimycin A (inhibitor of mitochondrial complex III, 10 μM), N^{G}-monomethyl-L-arginine acetate (L-NMMA, an inhibitor of NO synthases, 5 mM), or rotenone (inhibitor of mitochondria complex 1, 2 nM) as indicated. At 16 hours post-infection, the relative luciferase activity was measured. Values represent mean+/−s.e.m. (n=3). Significant differences were analyzed using the Student t test for the marked comparisons (†, *, p<0.001; for all other comparisons p value is given).

FIG. 4. H₂O₂ induces conformational changes in the AAV2 capsid accompanied by a sulfonic modification of a single cysteine residue in the capsid. 10¹⁰ purified virions of AAV2 were treated with (A, D) control buffer, (B, E) heat denatured at 70° C. for 5 minutes, (C, F) treated with 100 nM or (G) 1,000 nM H₂O₂ for 15 minutes, prior to overnight trypsin digestion at 37° C., DTT treatment and iodoacetamide labeling, and MALDI-TOF MS analysis. A-C) MALDI-TOF MS spectra (m/z range 1,000-4,000) of tryptic capsid peptides following the indicated treatments. D-G) Expanded MALDI-TOF MS spectra of the second cysteine on the AAV2 capORF (marked as a green colored diamond in H), which is located in the tryptic peptide FHCHFSPR (C289 relative to VP1 sequence). The detected m/z values for this peptide with different modifications on the cysteine residue are labeled at the top of the peaks. The theoretical m/z values are 1030.47 without modification, 1087.49 with iodoacetamide modification, and 1078.47 with sulfonic modification. H) The specific regions of AAV2 capsid exposed by H₂O₂-treatment are highlighted in different colors (blue, green, and pink) in the schematic illustration of the Cap ORFs. Arrows indicate the starting codons of VP1, 2 and 3; brown triangles: amino acid residues with proposed high surface accessibility²²; orange diamonds: location of cysteine residues. I) AAV2 virions treated with the indicated concentration of H₂O₂ for 15 minutes (lane 1-6) were assayed for PLA₂ activity using thin layer chromatography. Controls included heat-treated virions (lane 7), Bee venom PLA₂ (lane 8), intact untreated AAV2 virions (lane 9), buffer control (lane 10). Arrows indicate reaction products of PLA₂ cleavage (left) and a schematic structure of the C14-labeled (*) phosphatidylcholine precursor and products of cleavage are given to the right.

FIG. 5. Rac1, Nox2, AAV2 genomes, and exogenously loaded catalase all fractionate to the endosomal compartment following AAV2 infection. HeLa cells were treated with control medium (no virus or catalase) (left panel), medium containing AV2Luc (10³ particles/cell) (middle panel) or medium containing AV2Luc (10³ particles/cell) and catalase (1 mg/mL) (right panel) for 20 minutes. Cells were then homogenized and the PNS was loaded onto iodixanol-gradients for endosomal fractionation. Nox activity in each fraction was then determined using an NADPH-dependent lucigenin-based assay as described in the methods section. The amount of virus in each fraction was also determined by quantification of vector genomes using TaqMan PCR as described in the methods section. The Western blots at the bottom of each panel depict the distribution of catalase, Rac1, and Nox2 in each corresponding fraction. Vesicular fractions were concentrated by high-speed centrifugation at 100,000×g for 1 hour prior to SDS-PAGE and Western analysis.

FIG. 6. H₂O₂ induces conformational changes in the AAV2 capsid and sulfonic acid modification of a single cysteine residue in the capsid. 10¹⁰ purified virions of AAV2 were treated with (A, D) control buffer, (B, E) heat denatured at 70° C. for 5 minutes, (C, F) treated with 100 nM or (G) 1,000 nM H₂O₂ for 15 minutes, prior to overnight trypsin digestion at 37° C., DTT treatment and iodoacetamide labeling, and MALDI-TOF MS analysis. A-C) MALDI-TOF MS spectra (m/z range 1,000-4,000) of tryptic capsid peptides following the indicated treatments. D-G) Expanded MALDI-TOF MS spectra of the second cysteine on the AAV2 cap ORF (marked as a green colored diamond in H), which is located in the tryptic peptide FHCHFSPR (C289 relative to VP1 sequence). The detected m/z values for this peptide with different modifications on the cysteine residue are labeled at the top of the peaks. The theoretical m/z values are 1030.47 without modification, 1087.49 with iodoacetamide modification, and 1078.47 with sulfonic modification. H, The specific regions of AAV2 capsid exposed by H₂O₂-treatment are highlighted in different colors (blue, green, and pink) in the schematic illustration of the cap ORFs. Arrows indicate the starting codons of VP1, 2 and 3; brown triangles: amino acid residues with proposed high surface accessibility (Xie et al., 2002); orange diamonds: location of cysteine residues. I) AAV2 virions treated with the indicated concentration of H₂O₂ for 15 minutes (lanes 1-6) were assayed for PLA₂ activity. Controls included heat-treated virions (lane 7), Bee venom PLA₂ (lane 8), intact untreated AAV2 virions (lane 9), and buffer control (lane 10). Arrows indicate reaction products of PLA₂ cleavage (left) and a schematic structure of the C¹⁴-labeled (*) phosphatidylcholine precursor and products of cleavage are given to the right.

FIG. 7. Tryptic peptide masses of AAV capsid proteins liberated by H₂O₂ treatment. Following trypsin digestion and MALDI-TOF MS, the peptide masses visualized by MS in H₂O₂-treated virions (FIG. 6C), but not in the intact virions (FIG. 6A) are summarized. The parameters include their m/z values, exact amino acid sequences, and residue localizations on the cap ORF starting from VP1. The relative positions of these peptides are plotted on the schematic diagram of the cap ORFs (top) with corresponding colors. Arrows indicate the starting codons of VP1, 2, and 3; brown triangles: amino acid residues with proposed high surface accessibility; orange diamonds: location of cysteine residues.

FIG. 8. H₂O₂ induces exposure, but not oxidative modification, of C482 in the AAV2 capsid. 10¹⁰ purified virions of AAV2 were treated with (A) control buffer, (B) heat denatured at 70° C. for 5 minutes, (C) 100 nM H₂O₂ for 15 minutes, or (D) 1,000 nM H₂O₂ for 15 minutes, prior to overnight trypsin digestion at 37° C. in the presence of DTT, iodoacetamide labeling, and then MALDI-TOF MS analysis. MS spectra of the fifth cysteine in the AAV2 capORF (last orange colored diamond in FIG. 6H) are depicted. This cysteine is located in the tryptic peptide NWLPGPCYR (C482 relative to VP1 sequence). The corresponding signal for this peptide matched the expected m/z (1062.55) for iodoacetamide modification on cysteine C482 (marked by arrows). Expected m/z for the unmodified (1105.52, not marked) and sulfonic acid modified (1153.50, marked by arrow head) cysteine C482 in this peptide were not observed.

FIG. 9. The status of cysteine residues in AAV2 capsid following H₂O₂ treatment. The profiles of the corresponding AAV2 tryptic peptides that contain the individual cysteine residues are summarized. The parameters include their amino acid locations on the cap ORF as references from VP1, 2 and 3, the expected m/z value, and their detected m/z value. The conditions include intact (Ctrl), heat denatured (HD) or 100 nM H₂O₂ treated virions. N/D—not detected.

FIG. 10. H₂O₂-mediated capsid PLA₂ activation is essential for AAV2 endosomal escape. A) Top panel: Approach to separate free virions in the cytoplasm from virions inside endosomes. Bottom panel: HeLa cells (2×10⁷) were preincubated with AV2Luc (10³ MOI) for 1 hour at 4° C. followed by chasing infection at 37° C. for 1 hour. Cells were then homogenized and 500 μl PNS was collected. Free AAV2 virions mixed with PBS, free AAV2 virions mixed with PNS from uninfected cells, PNS from AAV2-infected cells, or AAV2-infected PNS incubated with 0.1% Triton X-100, was loaded to the top of 250 μL 30% iodixanol, followed by centrifugation at 100,000×g for 1 hour. Viral genome within the supernatant and pellet were quantified by real-time PCR and their corresponding percentage of total genomes are plotted. Values represent mean±s.e.m. (n=4). B) HeLa cells (2×10⁷) were preincubated with AV2Luc (10³ MOI) for 1 hour at 4° C. followed by chasing infection at 37° C. for the indicated period. Viral escape was then analyzed (n=5 in each time point; †P<0.001, *p<0.005). C) AV2Luc (10³ particles/cell) encapsidated in wild-type capsid or C289S capsid were used to infect HeLa cells in the presence of absence of 1 mg/mL catalase. Relative luciferase activity (left panel) and viral endosomal escape (right panel) was measured for each group at 24 hours and 1 hour post-viral infection, respectively. Values represent mean±s.e.m. (n=5), †*p<0.001. Significant differences were analyzed using the Student t test for the marked comparisons. D) AAV2 virions encapsidated in wild-type (W) or C289S (M) capsids were treated with the indicated concentration of H₂O₂ for 15 minutes (lanes 7-16), and assayed for PLA₂ activity using thin layer chromatography. Controls included Bee venom PLA₂ (lane 1), buffer control (lane 2), intact untreated AAV2 virions (lanes 3 and 4), heat-treated virions (lane 5 and 6). Arrows indicate reaction products of PLA₂ cleavage. The bottom panel shows the quantification of % substrate cleavage in each lane using a phosphoimager. Results are representative of three independent experiments.

FIG. 11. Isolation of redox-active endosomes containing Rac1. MCF-7 mammary epithelial cells were infected with a recombinant adenovirus expressing HA-tagged wtRac1 at a multiplicity of infection of 500 particles/cell. This level of infection gave rise to approximately 85% of the cells expressing transgene as previously reported (Li et al., 2005). 48 hours following adenovirus infection, cells were stimulated with IL-1β (1 ng/mL) for 20 minutes, and vesicular fractions were isolated as previously described (Li et al., 2005). Half of the crude combined vesicular peak #2-4 fractions was used for immuno-affinity isolation of HA-Rac1-associated endosomes using anti-HA bound Dynabeads using a procedure developed for isolation of HA-Rab5 endosomes (Li et al., 2005). The crude vesicular sample (V), immuno-isolated pellets (P), and supernatants (S) were evaluated for NADPH-dependent .O₂ production and Western blotting for the indicated proteins. Values for .O₂ production give the total activity in each sample (V, P, or S). An equal percentage of each sample (V, P, or S) was loaded in each Western blot lane.

### DETAILED DESCRIPTION OF THE INVENTION

### Definitions

A “vector” as used herein refers to a macromolecule or association of macromolecules that comprises or associates with a polynucleotide and which can be used to mediate delivery of the polynucleotide to a cell, either in vitro or in vivo. Illustrative vectors include, for example, plasmids, viral vectors, liposomes and other gene delivery vehicles. The polynucleotide to be delivered, sometimes referred to as a “target polynucleotide” or “transgene,” may comprise a coding sequence of interest in gene therapy (such as a gene encoding a protein of therapeutic or interest), a coding sequence of interest in vaccine development (such as a polynucleotide expressing a protein, polypeptide or peptide suitable for eliciting an immune response in a mammal), and/or a selectable or detectable marker.

“Parvovirus” is a family of viruses including Parovirus, Dependovirus and Densovirus. Adeno-associated virus is an exemplary parvovirus.

“AAV” is adeno-associated virus, and may be used to refer to the naturally occurring wild-type virus itself or derivatives thereof. The term covers all subtypes, serotypes and pseudotypes, and both naturally occurring and recombinant forms, except where required otherwise. As used herein, the term “serotype” refers to an AAV which is identified by and distinguished from other AAVs based on capsid protein reactivity with defined antisera, e.g., there are ten serotypes of primate AAVs, AAV-1 to AAV-10. For example, serotype AAV2 is used to refer to an AAV which contains capsid proteins encoded from the cap gene of AAV 2 and a genome containing 5′ and 3′ ITR sequences from the same AAV2 serotype. Pseudotyped AAV as refers to an AAV that contains capsid proteins from one serotype and a viral genome including 5′-3′ ITRs of a second serotype. Pseudotyped rAAV would be expected to have cell surface binding properties of the capsid serotype and genetic properties consistent with the ITR serotype. Pseudotyped rAAV are produced using standard techniques described in the art. As used herein, for example, rAAV5 may be used to refer an AAV having both capsid proteins and 5′-3′ ITRs from the same serotype or it may refer to an AAV having capsid proteins from serotype 5 and 5′-3′ ITRs from a different AAV serotype, e.g., AAV serotype 2. For each example illustrated herein the description of the vector design and production describes the serotype of the capsid and 5′-3′ ITR sequences. The abbreviation “rAAV” refers to recombinant adeno-associated virus, also referred to as a recombinant AAV vector (or “rAAV vector”).

“Transduction” or “transducing” as used herein, are terms referring to a process for the introduction of an exogenous polynucleotide by a viral vector, e.g., a transgene in rAAV vector, into a host cell leading to expression of the polynucleotide, e.g., the transgene in the cell. For instance, for AAV, the process includes 1) endocytosis of the AAV after it has bound to a cell surface receptor, 2) escape from endosomes or other intracellular compartments in the cytosol of a cell, 3) trafficking of the viral particle or viral genome to the nucleus, 4) uncoating of the virus particles, and generation of expressible double stranded AAV genome forms, including circular intermediates. The rAAV expressible double stranded form may persist as a nuclear episome or optionally may integrate into the host genome. The alteration of endosomal activation and/or endosomal residence time by an agent of the invention, may result in altered expression levels or persistence of expression, altered trafficking to the nucleus, altered types or relative numbers of host cells or a population of cells expressing the introduced polynucleotide, and/or altered virus production. Altered expression or persistence of a polynucleotide introduced via a virus can be determined by methods well known to the art including, but not limited to, protein expression, e.g., by ELISA, flow cytometry and Western blot, measurement of and DNA and RNA production by hybridization assays, e.g., Northern blots, Southern blots and gel shift mobility assays. In one embodiment, an agent of the invention enhances or increases NADPH oxidase activity, e.g., ROS production, which may alter endosomal processing or escape from endosomes or other intracellular cytosolic compartments, so as to alter expression of the introduced polynucleotide, e.g., a transgene in a rAAV vector, in vitro or in vivo. Methods used for the introduction of the exogenous polynucleotide include well-known techniques such as transfection, lipofection, viral infection, transformation, and electroporation, as well as non-viral gene delivery techniques. The introduced polynucleotide may be stably or transiently maintained in the host cell.

“Increased transduction or transduction frequency”, “altered transduction or transduction frequency”, or “enhanced transduction or transduction frequency” refers to an increase in one or more of the activities described above in a treated cell relative to an untreated cell. Agents of the invention which increase transduction efficiency may be determined by measuring the effect on one or more transduction activities, which may include measuring the expression of the transgene, measuring the function of the transgene, or determining the number of particles necessary to yield the same transgene effect compared to host cells not treated with the agents.

“Proteosome modulator” refers to an agent or class of agents which alter or enhance rAAV transduction or rAAV transduction frequencies by interacting with, binding to, or altering the function of, and/or trafficking or location of the proteosome. Proteosome modulators may have other cellular functions as described in the art, e.g., such as doxyrubicin, an antibiotic. In one embodiment, proteosome modulators do not include proteosome inhibitors, e.g., such as tripeptidyl aldehydes (Z-LLL or LLnL), agents that inhibit calpains, cathepsins, cysteine proteases, and/or chymotrypsin-like protease activity of proteasomes (Wagner et al., 2002; Young et al., 2000; Seisenberger et al., 2001).

“Generation of double stranded expressible forms” or “conversion of single to double strand rAAV genomes” refers to the process of replicating in the nucleus of an rAAV infected host cell a complimentary strand of the rAAV single stranded vector DNA genome and annealing of the complimentary strand to the vector genome to produce a double stranded DNA rAAV genome. Agents of the invention described herein to increase, alter, or enhance rAAV transduction include agents which increase the rate of nuclear transport or the steady state of single stranded viral DNA genomes in the nucleus which can drive gene conversion events via steady state mechanisms. For the purposes of the invention described herein, agents which enhance conversion of single to double strands do not include agents which increase the concentration of DNA repair enzymes or activate alternate DNA repair mechanism described by Russel et al. (1995).

“Gene delivery” refers to the introduction of an exogenous polynucleotide into a cell for gene transfer, and may encompass targeting, binding, uptake, transport, localization, replicon integration and expression.

“Gene transfer” refers to the introduction of an exogenous polynucleotide into a cell which may encompass targeting, binding, uptake, transport, localization and replicon integration, but is distinct from and does not imply subsequent expression of the gene.

“Gene expression” or “expression” refers to the process of gene transcription, translation, and post-translational modification.

A “detectable marker gene” is a gene that allows cells carrying the gene to be specifically detected (e.g., distinguished from cells which do not carry the marker gene). A large variety of such marker genes are known in the art.

A “selectable marker gene” is a gene that allows cells carrying the gene to be specifically selected for or against, in the presence of a corresponding selective agent. By way of illustration, an antibiotic resistance gene can be used as a positive selectable marker gene that allows a host cell to be positively selected for in the presence of the corresponding antibiotic. A variety of positive and negative selectable markers are known in the art, some of which are described below.

An “rAAV vector” as used herein refers to an AAV vector comprising a polynucleotide sequence not of AAV origin (i.e., a polynucleotide heterologous to AAV), typically a sequence of interest for the genetic transformation of a cell. In preferred vector constructs of this invention, the heterologous polynucleotide is flanked by at least one, preferably two AAV inverted terminal repeat sequences (ITRs). The term rAAV vector encompasses both rAAV vector particles and rAAV vector plasmids.

An “AAV virus” or “AAV viral particle” refers to a viral particle composed of at least one AAV capsid protein (preferably by all of the capsid proteins of a wild-type AAV) and an encapsidated polynucleotide. If the particle comprises a heterologous polynucleotide (i.e., a polynucleotide other than a wild-type AAV genome such as a transgene to be delivered to a mammalian cell), it is typically referred to as “rAAV”.

A “viral vaccine” as used herein refers to a viral vector comprising a polynucleotide heterologous to that virus, that encodes a peptide, polypeptide, or protein capable of eliciting an immune response in a host contacted with the vector. Expression of the polynucleotide may result in generation of a neutralizing antibody response and/or a cell mediated response, e.g., a cytotoxic T cell response.

A “helper virus” for AAV refers to a virus that allows AAV (e.g., wild-type AAV) to be replicated and packaged by a mammalian cell. A variety of such helper viruses for AAV are known in the art, including adenoviruses, herpes viruses and poxviruses such as vaccinia. The adenoviruses encompass a number of different subgroups, although Adenovirus type 5 of subgroup C is most commonly used. Numerous adenoviruses of human, non-human mammalian and avian origin are known and available from depositories such as the ATCC. Viruses of the herpes family include, for example, herpes simplex viruses (HSV) and Epstein-Barr viruses (EBV), as well as cytomegaloviruses (CMV) and pseudorabies viruses (PRV); which are also available from depositories such as ATCC.

An “infectious” virus or viral particle is one that comprises a polynucleotide component which it is capable of delivering into a cell for which the viral species is trophic. The term does not necessarily imply any replication capacity of the virus.

A “replication-competent” virus (e.g., a replication-competent AAV, sometimes abbreviated as “RCA”) refers to a phenotypically wild-type virus that is infectious, and is also capable of being replicated in an infected cell (i.e., in the presence of a helper virus or helper virus functions). In the case of AAV, replication competence generally requires the presence of functional AAV packaging genes. Preferred rAAV vectors as described herein are replication-incompetent in mammalian cells (especially in human cells) by virtue of the lack of one or more AAV packaging genes. Preferably, such rAAV vectors lack any AAV packaging gene sequences in order to minimize the possibility that RCA are generated by recombination between AAV packaging genes and an incoming rAAV vector. Preferred rAAV vector preparations as described herein are those which contain few if any RCA (preferably less than about 1 RCA per 10² rAAV particles, more preferably less than about 1 RCA per 10⁴ RAAV particles, still more preferably less than about 1 RCA per 10⁸ rAAV particles, even more preferably less than about 1 RCA per 10¹² rAAV particles, most preferably no RCA).

The term “polynucleotide” refers to a polymeric form of nucleotides of any length, including deoxyribonucleotides or ribonucleotides, or analogs thereof. A polynucleotide may comprise modified nucleotides, such as methylated or capped nucleotides and nucleotide analogs, and may be interrupted by non-nucleotide components. If present, modifications to the nucleotide structure may be imparted before or after assembly of the polymer. The term polynucleotide, as used herein, refers interchangeably to double- and single-stranded molecules. Unless otherwise specified or required, any embodiment of the invention described herein that is a polynucleotide encompasses both the double-stranded form and each of two complementary single-stranded forms known or predicted to make up the double-stranded form.

A “gene” refers to a polynucleotide containing at least one open reading frame that is capable of encoding a particular protein after being transcribed and translated.

“Recombinant,” as applied to a polynucleotide means that the polynucleotide is the product of various combinations of cloning, restriction and/or ligation steps, and other procedures that result in a construct that is distinct from a polynucleotide found in nature. A recombinant virus is a viral particle comprising a recombinant polynucleotide. The terms respectively include replicates of the original polynucleotide construct and progeny of the original virus construct. A “control element” or “control sequence” is a nucleotide sequence involved in an interaction of molecules that contributes to the functional regulation of a polynucleotide, including replication, duplication, transcription, splicing, translation, or degradation of the polynucleotide. The regulation may affect the frequency, speed, or specificity of the process, and may be enhancing or inhibitory in nature. Control elements known in the art include, for example, transcriptional regulatory sequences such as promoters and enhancers. A promoter is a DNA region capable under certain conditions of binding RNA polymerase and initiating transcription of a coding region usually located downstream (in the 3′ direction) from the promoter. Promoters include AAV promoters, e.g., P5, P19, P40 and AAV ITR promoters, as well as heterologous promoters.

An “expression vector” is a vector comprising a region which encodes a polypeptide of interest, and is used for effecting the expression of the protein in an intended target cell. An expression vector also comprises control elements operatively linked to the encoding region to facilitate expression of the protein in the target. The combination of control elements and a gene or genes to which they are operably linked for expression is sometimes referred to as an “expression cassette,” a large number of which are known and available in the art or can be readily constructed from components that are available in the art.

“Genetic alteration” refers to a process wherein a genetic element is introduced into a cell other than by mitosis or meiosis. The element may be heterologous to the cell, or it may be an additional copy or improved version of an element already present in the cell. Genetic alteration may be effected, for example, by transfecting a cell with a recombinant plasmid or other polynucleotide through any process known in the art, such as electroporation, calcium phosphate precipitation, or contacting with a polynucleotide-liposome complex. Genetic alteration may also be effected, for example, by transduction or infection with a DNA or RNA virus or viral vector. Preferably, the genetic element is introduced into a chromosome or mini-chromosome in the cell; but any alteration that changes the phenotype and/or genotype of the cell and its progeny is included in this term. A cell is said to be “stably” altered, transduced or transformed with a genetic sequence if the sequence is available to perform its function during extended culture of the cell in vitro. In preferred examples, such a cell is “inheritably” altered in that a genetic alteration is introduced which is also inheritable by progeny of the altered cell.

The term “recombinant DNA molecule” as used herein refers to a DNA molecule that is comprised of segments of DNA joined together by means of molecular biological techniques.

A “transcriptional regulatory sequence” or “TRS,” as used herein, refers to a genomic region that controls the transcription of a gene or coding sequence to which it is operably linked. Transcriptional regulatory sequences of use in the present invention generally include at least one transcriptional promoter and may also include one or more enhancers and/or terminators of transcription. “Operably linked” refers to an arrangement of two or more components, wherein the components so described are in a relationship permitting them to function in a coordinated manner. By way of illustration, a transcriptional regulatory sequence or a promoter is operably linked to a coding sequence if the TRS or promoter promotes transcription of the coding sequence. An operably linked TRS is generally joined in cis with the coding sequence, but it is not necessarily directly adjacent to it.

A “terminator” refers to a polynucleotide sequence that tends to diminish or prevent read-through transcription (i.e., it diminishes or prevent transcription originating on one side of the terminator from continuing through to the other side of the terminator). The degree to which transcription is disrupted is typically a function of the base sequence and/or the length of the terminator sequence. In particular, as is well known in numerous molecular biological systems, particular DNA sequences, generally referred to as “transcriptional termination sequences” are specific sequences that tend to disrupt read-through transcription by RNA polymerase, presumably by causing the RNA polymerase molecule to stop and/or disengage from the DNA being transcribed. Typical example of such sequence-specific terminators include polyadenylation (“polyA”) sequences, e.g., SV40 polyA. In addition to or in place of such sequence-specific terminators, insertions of relatively long DNA sequences between a promoter and a coding region also tend to disrupt transcription of the coding region, generally in proportion to the length of the intervening sequence. This effect presumably arises because there is always some tendency for an RNA polymerase molecule to become disengaged from the DNA being transcribed, and increasing the length of the sequence to be traversed before reaching the coding region would generally increase the likelihood that disengagement would occur before transcription of the coding region was completed or possibly even initiated. Terminators may thus prevent transcription from only one direction (“uni-directional” terminators) or from both directions (“bi-directional” terminators), and may be comprised of sequence-specific termination sequences or sequence-non-specific terminators or both. A variety of such terminator sequences are known in the art; and illustrative uses of such sequences within the context of the present invention are provided below.

The term “polypeptide” and protein” are used interchangeably herein unless otherwise distinguished, to refer to polymers of amino acids of any length. The terms also encompass an amino acid polymer that has been modified; for example, disulfide bond formation, glycosylation, acetylation, phosphonylation, lipidation, or conjugation with a labeling component. Polypeptides such as “CFTR” and the like, when discussed in the context of gene therapy and compositions therefor, refer to the respective intact polypeptide, or any fragment or genetically engineered derivative thereof, that retains the desired biochemical function of the intact protein. Similarly, references to CFTR, and other such genes for use in gene therapy (typically referred to as “transgenes” to be delivered to a recipient cell), include polynucleotides encoding the intact polypeptide or any fragment or genetically engineered derivative possessing the desired biochemical function.

The term “recombinant protein” or “recombinant polypeptide” as used herein refers to a protein molecule that is expressed from a recombinant DNA molecule.

The term “isolated” when used in relation to a nucleic acid, peptide, polypeptide or virus refers to a nucleic acid sequence, peptide, polypeptide or virus that is identified and separated from at least one contaminant nucleic acid, polypeptide, virus or other biological component with which it is ordinarily associated in its natural source. For example, an isolated substance may be prepared by using a purification technique to enrich it from a source mixture. Enrichment can be measured on an absolute basis, such as weight per volume of solution, or it can be measured in relation to a second, potentially interfering substance present in the source mixture. Increasing enrichments of the embodiments of this invention are increasingly more preferred. For example, a 2-fold enrichment is preferred, 10-fold enrichment is more preferred, 100-fold enrichment is more preferred, 1000-fold enrichment is even more preferred. Thus, isolated nucleic acid, peptide, polypeptide or virus is present in a form or setting that is different from that in which it is found in nature. For example, a given DNA sequence (e.g., a gene) is found on the host cell chromosome in proximity to neighboring genes; RNA sequences, such as a specific mRNA sequence encoding a specific protein, are found in the cell as a mixture with numerous other mRNAs that encode a multitude of proteins. The isolated nucleic acid molecule may be present in single-stranded or double-stranded form. When an isolated nucleic acid molecule is to be utilized to express a protein, the molecule will contain at a minimum the sense or coding strand (i.e., the molecule may single-stranded), but may contain both the sense and anti-sense strands (i.e., the molecule may be double-stranded).

“Heterologous” means derived from a genotypically distinct entity from that of the rest of the entity to which it is compared. For example, a polynucleotide introduced by genetic engineering techniques into a different cell type is a heterologous polynucleotide (and, when expressed, can encode a heterologous polypeptide). Similarly, a TRS or promoter that is removed from its native coding sequence and operably linked to a different coding sequence is a heterologous TRS or promoter.

The term “exogenous,” when used in relation to a protein, gene, nucleic acid, or polynucleotide in a cell or organism refers to a protein, gene, nucleic acid, or polynucleotide which has been introduced into the cell or organism by artificial or natural means. An exogenous nucleic acid may be from a different organism or cell, or it may be one or more additional copies of a nucleic acid which occurs naturally within the organism or cell. By way of a non-limiting example, an exogenous nucleic acid is in a chromosomal location different from that of natural cells, or is otherwise flanked by a different nucleic acid sequence than that found in nature, e.g., an expression cassette which links a promoter from one gene to an open reading frame for a gene product from a different gene.

The term “sequence homology” means the proportion of base matches between two nucleic acid sequences or the proportion amino acid matches between two amino acid sequences. When sequence homology is expressed as a percentage, e.g., 50%, the percentage denotes the proportion of matches over the length of a selected sequence that is compared to some other sequence. Gaps (in either of the two sequences) are permitted to maximize matching; gap lengths of 15 bases or less are usually used, 6 bases or less are preferred with 2 bases or less more preferred. When using oligonucleotides as probes or treatments, the sequence homology between the target nucleic acid and the oligonucleotide sequence is generally not less than 17 target base matches out of 20 possible oligonucleotide base pair matches (85%); preferably not less than 9 matches out of 10 possible base pair matches (90%), and more preferably not less than 19 matches out of 20 possible base pair matches (95%).

Two amino acid sequences are homologous if there is a partial or complete identity between their sequences. For example, 85% homology means that 85% of the amino acids are identical when the two sequences are aligned for maximum matching. Gaps (in either of the two sequences being matched) are allowed in maximizing matching; gap lengths of 5 or less are preferred with 2 or less being more preferred. Alternatively and preferably, two protein sequences (or polypeptide sequences derived from them of at least 30 amino acids in length) are homologous, as this term is used herein, if they have an alignment score of at more than 5 (in standard deviation units) using the program ALIGN with the mutation data matrix and a gap penalty of 6 or greater. See Dayhoff, 1972. The two sequences or parts thereof are more preferably homologous if their amino acids are greater than or equal to 50% identical when optimally aligned using the ALIGN program.

The term “corresponds to” is used herein to mean that a polynucleotide sequence is structurally related to all or a portion of a reference polynucleotide sequence, or that a polypeptide sequence is structurally related to all or a portion of a reference polypeptide sequence, e.g., they have at least 80%, 85%, 90%, 95% or more, e.g., 99% or 100%, sequence identity. In contradistinction, the term “complementary to” is used herein to mean that the complementary sequence is homologous to all or a portion of a reference polynucleotide sequence. For illustration, the nucleotide sequence “TATAC” corresponds to a reference sequence “TATAC” and is complementary to a reference sequence “GTATA”.

The following terms are used to describe the sequence relationships between two or more polynucleotides: “reference sequence”, “comparison window”, “sequence identity”, “percentage of sequence identity”, and “substantial identity”. A “reference sequence” is a defined sequence used as a basis for a sequence comparison; a reference sequence may be a subset of a larger sequence, for example, as a segment of a full-length cDNA or gene sequence given in a sequence listing, or may comprise a complete cDNA or gene sequence. Generally, a reference sequence is at least 20 nucleotides in length, frequently at least 25 nucleotides in length, and often at least 50 nucleotides in length. Since two polynucleotides may each (1) comprise a sequence (i.e., a portion of the complete polynucleotide sequence) that is similar between the two polynucleotides, and (2) may further comprise a sequence that is divergent between the two polynucleotides, sequence comparisons between two (or more) polynucleotides are typically performed by comparing sequences of the two polynucleotides over a “comparison window” to identify and compare local regions of sequence similarity.

A “comparison window”, as used herein, refers to a conceptual segment of at least 20 contiguous nucleotides and wherein the portion of the polynucleotide sequence in the comparison window may comprise additions or deletions (i.e., gaps) of 20 percent or less as compared to the reference sequence (which does not comprise additions or deletions) for optimal alignment of the two sequences. Optimal alignment of sequences for aligning a comparison window may be conducted by the local homology algorithm of Smith and Waterman (1981), by the homology alignment algorithm of Needleman and Wunsch (1970), by the search for similarity method of Pearson and Lipman (1988), by computerized implementations of these algorithms (GAP, BESTFIT, FASTA, and TFASTA in the Wisconsin Genetics Software Package Release 7.0, Genetics Computer Group, 575 Science Dr., Madison, Wis.), or by inspection, and the best alignment (i.e., resulting in the highest percentage of homology over the comparison window) generated by the various methods is selected.

The term “sequence identity” means that two polynucleotide sequences are identical (i.e., on a nucleotide-by-nucleotide basis) over the window of comparison. The term “percentage of sequence identity” means that two polynucleotide sequences are identical (i.e., on a nucleotide-by-nucleotide basis) over the window of comparison. The term “percentage of sequence identity” is calculated by comparing two optimally aligned sequences over the window of comparison, determining the number of positions at which the identical nucleic acid base (e.g., A, T, C, G, U, or I) occurs in both sequences to yield the number of matched positions, dividing the number of matched positions by the total number of positions in the window of comparison (i.e., the window size), and multiplying the result by 100 to yield the percentage of sequence identity. The terms “substantial identity” as used herein denote a characteristic of a polynucleotide sequence, wherein the polynucleotide comprises a sequence that has at least 85 percent sequence identity, preferably at least 90 to 95 percent sequence identity, more usually at least 99 percent sequence identity as compared to a reference sequence over a comparison window of at least 20 nucleotide positions, frequently over a window of at least 20-50 nucleotides, wherein the percentage of sequence identity is calculated by comparing the reference sequence to the polynucleotide sequence which may include deletions or additions which total 20 percent or less of the reference sequence over the window of comparison.

As applied to polypeptides, the term “substantial identity” means that two peptide sequences, when optimally aligned, such as by the programs GAP or BESTFIT using default gap weights, share at least about 80 percent sequence identity, preferably at least about 90 percent sequence identity, more preferably at least about 95 percent sequence identity, and most preferably at least about 99 percent sequence identity.

“Packaging” as used herein refers to a series of subcellular events that results in the assembly and encapsidation of a viral vector. Thus, when a suitable vector is introduced into a packaging cell line under appropriate conditions, it can be assembled into a viral particle.

“Host cells,” “cell lines,” “cell cultures,” “packaging cell line” and other such terms denote higher eukaryotic cells, preferably mammalian cells, most preferably human cells, useful in the present invention. These cells can be used as recipients for recombinant vectors, viruses or other transfer polynucleotides, and include the progeny of the original cell that was transduced. It is understood that the progeny of a single cell may not necessarily be completely identical (in morphology or in genomic complement) to the original parent cell.

“Transfected,” “transformed” or “transgenic” is used herein to include any host cell or cell line, which has been altered or augmented by the presence of at least one recombinant DNA sequence.

A “therapeutic gene,” “prophylactic gene,” “target polynucleotide,” “transgene,” “gene of interest” and the like generally refer to a gene or genes to be transferred using a vector. Typically, in the context of the present invention, such genes are located within the viral vector (which can be replicated and encapsidated into particles). Target polynucleotides can be used in this invention to generate vectors for a number of different applications. Such polynucleotides include, but are not limited to: (i) polynucleotides encoding proteins useful in other forms of gene therapy to relieve deficiencies caused by missing, defective or sub-optimal levels of a structural protein or enzyme; (ii) polynucleotides that are transcribed into anti-sense molecules; (iii) polynucleotides that are transcribed into decoys that bind transcription or translation factors; (iv) polynucleotides that encode cellular modulators such as cytokines; (v) polynucleotides that can make recipient cells susceptible to specific drugs, such as the herpes virus thymidine kinase gene; and (vi) polynucleotides for cancer therapy, such as E1A tumor suppressor genes or p53 tumor suppressor genes for the treatment of various cancers. To effect expression of the transgene in a recipient host cell, it is preferably operably linked to a promoter, either its own or a heterologous promoter. A large number of suitable promoters are known in the art, the choice of which depends on the desired level of expression of the target polynucleotide; whether one wants constitutive expression, inducible expression, cell-specific or tissue-specific expression, etc. The viral vector may also contain a selectable marker.

A preparation of AAV is said to be “substantially free” of helper virus if the ratio of infectious AAV particles to infectious helper virus particles is at least about 10²:1; preferably at least about 10⁴:1, more preferably at least about 10⁶:1; still more preferably at least about 10⁸:1. Preparations are also preferably free of equivalent amounts of helper virus proteins (i.e., proteins as would be present as a result of such a level of helper virus if the helper virus particle impurities noted above were present in disrupted form). Viral and/or cellular protein contamination can generally be observed as the presence of Coomassie staining bands on SDS gels (e.g., the appearance of bands other than those corresponding to the AAV capsid proteins VP1, VP2 and VP3).

“Efficiency” when used in describing viral production, replication or packaging refers to useful properties of the method: in particular, the growth rate and the number of virus particles produced per cell. “High efficiency” production indicates production of at least 100 viral particles per cell; preferably at least about 10,000 and more preferably at least about 100,000 particles per cell, over the course of the culture period specified.

An “individual” or “subject” treated in accordance with this invention refers to vertebrates, particularly members of a mammalian species, and includes but is not limited to domestic animals, sports animals, and primates, including humans. “Treatment” of an individual or a cell is any type of intervention in an attempt to alter the natural course of the individual or cell at the time the treatment is initiated, e.g., eliciting a prophylactic, curative or other beneficial effect in the individual. As used herein, “treating” or “treat” includes (i) preventing a pathologic condition from occurring (e.g. prophylaxis); (ii) inhibiting the pathologic condition or arresting its development; (iii) relieving the pathologic condition; and/or diminishing symptoms associated with the pathologic condition. For example, treatment of an individual may be undertaken to decrease or limit the pathology caused by any pathological condition, including (but not limited to) an inherited or induced genetic deficiency, infection by a viral, bacterial, or parasitic organism, a neoplastic or aplastic condition, or an immune system dysfunction such as autoimmunity or immunosuppression. Treatment includes (but is not limited to) administration of a composition, such as a pharmaceutical composition, and administration of compatible cells that have been treated with a composition. Treatment may be performed either prophylactically or therapeutically; that is, either prior or subsequent to the initiation of a pathologic event or contact with an etiologic agent.

As used herein, “substantially pure” or “purified” means an object species is the predominant species present (i.e., on a molar basis it is more abundant than any other individual species in the composition), and preferably a substantially purified fraction is a composition wherein the object species comprises at least about 50 percent (on a molar basis) of all macromolecular species present. Generally, a substantially pure composition will comprise more than about 80 percent of all macromolecular species present in the composition, more preferably more than about 85%, about 90%, about 95%, and about 99%. Most preferably, the object species is purified to essential homogeneity (contaminant species cannot be detected in the composition by conventional detection methods) wherein the composition consists essentially of a single macromolecular species.

As used herein, “pharmaceutically acceptable salts” refer to derivatives of the disclosed compounds wherein the parent compound is modified by making acid or base salts thereof. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, and the like.

The pharmaceutically acceptable salts of compounds useful in the present invention can be synthesized from the parent compound, which contains a basic or acidic moiety, by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences (1985), the disclosure of which is hereby incorporated by reference.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication commensurate with a reasonable benefit/risk ratio.

One diastereomer of a compound disclosed herein may display superior activity compared with the other. When required, separation of the racemic material can be achieved by HPLC using a chiral column or by a resolution using a resolving agent such as camphonic chloride. A chiral compound of Formula I may also be directly synthesized using a chiral catalyst or a chiral ligand.

“Therapeutically effective amount” is intended to include an amount of a compound useful in the present invention or an amount of the combination of compounds claimed, e.g., to treat or prevent the disease or disorder, or to treat the symptoms of the disease or disorder, in a host. The combination of compounds is preferably a synergistic combination. Synergy occurs when the effect of the compounds when administered in combination is greater than the additive effect of the compounds when administered alone as a single agent. In general, a synergistic effect is most clearly demonstrated at suboptimal concentrations of the compounds. Synergy can be in terms of lower cytotoxicity, increased activity, or some other beneficial effect of the combination compared with the individual components.

“Stable compound” and “stable structure” are meant to indicate a compound that is sufficiently robust to survive isolation to a useful degree of purity from a reaction mixture, and formulation into an efficacious therapeutic agent. Only stable compounds are contemplated by the present invention.

“Substituted” is intended to indicate that one or more hydrogens on the atom indicated in the expression using “substituted” is replaced with a selection from the indicated group(s), provided that the indicated atom's normal valency is not exceeded, and that the substitution results in a stable compound. Suitable indicated groups include, e.g., alkyl, alkenyl alkoxy, halo, haloalkyl, hydroxy, hydroxyalkyl, aryl, heteroaryl, heterocycle, cycloalkyl, alkanoyl, alkoxycarbonyl, amino, imino, alkylamino, acylamino, nitro, trifluoromethyl, trifluoromethoxy, carboxy, carboxyalkyl, keto, thioxo, alkylthio, alkylsulfinyl, alkylsulfonyl, cyano, NR^{x}R^{y} and/or COOR^{x}, wherein each R^{x} and R^{y} are independently H, alkyl, alkenyl, aryl, heteroaryl, heterocycle, cycloalkyl or hydroxy. When a substituent is keto (i.e., ═O) or thioxo (i.e., ═S) group, then 2 hydrogens on the atom are replaced.

“Interrupted” is intended to indicate that in between two or more adjacent carbon atoms, and the hydrogen atoms to which they are attached (e.g., methyl (CH₃), methylene (CH₂) or methine (CH)), indicated in the expression using “interrupted” is inserted with a selection from the indicated group(s), provided that the each of the indicated atoms' normal valency is not exceeded, and that the interruption results in a stable compound. Such suitable indicated groups include, e.g., non-peroxide oxy (—O—), thio (—S—), carbonyl (—C(═O)—), carboxy (—C(═O)—), imine (C═NH), sulfonyl (SO) or sulfoxide (SO₂).

Specific and preferred values listed below for radicals, substituents, and ranges, are for illustration only; they do not exclude other defined values or other values within defined ranges for the radicals and substituents

“Alkyl” refers to a C₁-C₁₈ hydrocarbon containing normal, secondary, tertiary or cyclic carbon atoms. Examples are methyl (Me, —CH₃), ethyl (Et, —CH₂CH₃), 1-propyl (n-Pr, n-propyl, —CH₂CH₂CH₃), 2-propyl (i-Pr, i-propyl, —CH(CH₃)₂), 1-butyl (n-Bu, n-butyl, —CH₂CH₂CH₂CH₃), 2-methyl-1-propyl (i-Bu, i-butyl, —CH₂CH(CH₃)₂), 2-butyl (s-Bu, s-butyl, —CH(CH₃)CH₂CH₃), 2-methyl-2-propyl (t-Bu, t-butyl, —C(CH₃)₃), 1-pentyl (n-pentyl, —CH₂CH₂CH₂CH₂CH₃), 2-pentyl (—CH(CH₃)CH₂CH₂CH₃), 3-pentyl (—CH(CH₂CH₃)₂), 2-methyl-2-butyl (—C(CH₃)₂CH₂CH₃), 3-methyl-2-butyl (—CH(CH₃)CH(CH₃)₂), 3-methyl-1-butyl (—CH₂CH₂CH(CH₃)₂), 2-methyl-1-butyl (—CH₂CH(CH₃)CH₂CH₃), 1-hexyl (—CH₂CH₂CH₂CH₂CH₂CH₃), 2-hexyl (—CH(CH₃)CH₂CH₂CH₂CH₃), 3-hexyl (—CH(CH₂CH₃)(CH₂CH₂CH₃)), 2-methyl-2-pentyl (—C(CH₃)₂CH₂CH₂CH₃), 3-methyl-2-pentyl (—CH(CH₃)CH(CH₃)CH₂CH₃), 4-methyl-2-pentyl (—CH(CH₃)CH₂CH(CH₃)₂), 3-methyl-3-pentyl (—C(CH₃)(CH₂CH₃)₂), 2-methyl-3-pentyl (—CH(CH₂CH₃)CH(CH₃)₂), 2,3-dimethyl-2-butyl (—C(CH₃)₂CH(CH₃)₂), 3,3-dimethyl-2-butyl (—CH(CH₃)C(CH₃)₃.

The alkyl can optionally be substituted with one or more alkenyl alkoxy, halo, haloalkyl, hydroxy, hydroxyalkyl, aryl, heteroaryl, heterocycle, cycloalkyl, alkanoyl, alkoxycarbonyl, amino, imino, alkylamino, acylamino, nitro, trifluoromethyl, trifluoromethoxy, carboxy, carboxyalkyl, keto, thioxo, alkylthio, alkylsulfinyl, alkylsulfonyl, cyano, NR^{x}R^{y} and/or COOR^{x}, wherein each R^{x} and R^{y} are independently H, alkyl, alkenyl, aryl, heteroaryl, heterocycle, cycloalkyl or hydroxyl. The alkyl can optionally be interrupted with one or more non-peroxide oxy (—O—), thio (—S—), carbonyl (—C(═O)—), carboxy (—C(═O)O—), sulfonyl (SO) or sulfoxide (SO₂). Additionally, the alkyl can optionally be at least partially unsaturated, thereby providing an alkenyl.

“Alkenyl” refers to a C₂-C₁₈ hydrocarbon containing normal, secondary, tertiary or cyclic carbon atoms with at least one site of unsaturation, i.e., a carbon-carbon, sp² double bond. Examples include, but are not limited to: ethylene or vinyl (—CH═CH₂), allyl (—CH₂CH═CH₂), cyclopentenyl (—C₅H₇), and 5-hexenyl (—CH₂ CH₂CH₂CH₂CH═CH₂).

The alkenyl can optionally be substituted with one or more alkyl alkoxy, halo, haloalkyl, hydroxy, hydroxyalkyl, aryl, heteroaryl, heterocycle, cycloalkyl, alkanoyl, alkoxycarbonyl, amino, imino, alkylamino, acylamino, nitro, trifluoromethyl, trifluoromethoxy, carboxy, carboxyalkyl, keto, thioxo, alkylthio, alkylsulfinyl, alkylsulfonyl, cyano, NR^{x}R^{y} and/or COOR^{x}, wherein each R^{x} and R^{y} are independently H, alkyl, alkenyl, aryl, heteroaryl, heterocycle, cycloalkyl or hydroxyl. Additionally, the alkenyl can optionally be interrupted with one or more non-peroxide oxy (—O—), thio (—S—), carbonyl (—C(═O)—), carboxy (—C(═O)O—), sulfonyl (SO) or sulfoxide (SO₂).

“Alkylene” refers to a saturated, branched or straight chain or cyclic hydrocarbon radical of 1-18 carbon atoms, and having two monovalent radical centers derived by the removal of two hydrogen atoms from the same or different carbon atoms of a parent alkane. Typical alkylene radicals include, but are not limited to: methylene (—CH₂—) 1,2-ethyl (—CH₂CH₂—), 1,3-propyl (—CH₂CH₂CH₂—), 1,4-butyl (—CH₂CH₂CH₂CH₂—), and the like.

The alkylene can optionally be substituted with one or more alkyl, alkenyl alkoxy, halo, haloalkyl, hydroxy, hydroxyalkyl, aryl, heteroaryl, heterocycle, cycloalkyl, alkanoyl, alkoxycarbonyl, amino, imino, alkylamino, acylamino, nitro, trifluoromethyl, trifluoromethoxy, carboxy, carboxyalkyl, keto, thioxo, alkylthio, alkylsulfinyl, alkylsulfonyl, cyano, NR^{x}R^{y} and/or COOR^{x}, wherein each R^{x} and R^{y} are independently H, alkyl, alkenyl, aryl, heteroaryl, heterocycle, cycloalkyl or hydroxyl. Additionally, the alkylene can optionally be interrupted with one or more non-peroxide oxy (—O—), thio (—S—), carbonyl (—C(═O)—), carboxy (—C(═O)O—), sulfonyl (SO) or sulfoxide (SO₂). Moreover, the alkylene can optionally be at least partially unsaturated, thereby providing an alkenylene.

“Alkenylene” refers to an unsaturated, branched or straight chain or cyclic hydrocarbon radical of 2-18 carbon atoms, and having two monovalent radical centers derived by the removal of two hydrogen atoms from the same or two different carbon atoms of a parent alkene. Typical alkenylene radicals include, but are not limited to: 1,2-ethylene (—CH═CH—).

The alkenylene can optionally be substituted with one or more alkyl, alkenyl alkoxy, halo, haloalkyl, hydroxy, hydroxyalkyl, aryl, heteroaryl, heterocycle, cycloalkyl, alkanoyl, alkoxycarbonyl, amino, imino, alkylamino, acylamino, nitro, trifluoromethyl, trifluoromethoxy, carboxy, carboxyalkyl, keto, thioxo, alkylthio, alkylsulfinyl, alkylsulfonyl, cyano, NR^{x}R^{y} and/or COOR^{x}, wherein each R^{x} and R^{y} are independently H, alkyl, alkenyl, aryl, heteroaryl, heterocycle, cycloalkyl or hydroxyl. Additionally, The alkenylene can optionally be interrupted with one or more non-peroxide oxy (—O—), thio (—S—), carbonyl (—C(═O)—), carboxy (—C(═O)O—), sulfonyl (SO) or sulfoxide (SO₂).

The term “alkoxy” refers to the groups alkyl-O—, where alkyl is defined herein. Preferred alkoxy groups include, e.g., methoxy, ethoxy, n-propoxy, iso-propoxy, n-butoxy, tert-butoxy, sec-butoxy, n-pentoxy, n-hexoxy, 1,2-dimethylbutoxy, and the like.

The alkoxy can optionally be substituted with one or more alkyl halo, haloalkyl, hydroxy, hydroxyalkyl, aryl, heteroaryl, heterocycle, cycloalkyl, alkanoyl, alkoxycarbonyl, amino, imino, alkylamino, acylamino, nitro, trifluoromethyl, trifluoromethoxy, carboxy, carboxyalkyl, keto, thioxo, alkylthio, alkylsulfinyl, alkylsulfonyl, cyano, NR^{x}R^{y} and COOR^{x}, wherein each R^{x} and R^{y} are independently H, alkyl, aryl, heteroaryl, heterocycle, cycloalkyl or hydroxyl.

The term “aryl” refers to an unsaturated aromatic carbocyclic group of from 6 to 20 carbon atoms having a single ring (e.g., phenyl) or multiple condensed (fused) rings, wherein at least one ring is aromatic (e.g., naphthyl, dihydrophenanthrenyl, fluorenyl, or anthryl). Preferred aryls include phenyl, naphthyl and the like.

The aryl can optionally be substituted with one or more alkyl, alkenyl, alkoxy, halo, haloalkyl, hydroxy, hydroxyalkyl, heteroaryl, heterocycle, cycloalkyl, alkanoyl, alkoxycarbonyl, amino, imino, alkylamino, acylamino, nitro, trifluoromethyl, trifluoromethoxy, carboxy, carboxyalkyl, keto, thioxo, alkylthio, alkylsulfinyl, alkylsulfonyl, cyano, NR^{x}R^{y} and COOR^{x}, wherein each R^{x} and R^{y} are independently H, alkyl, aryl, heteroaryl, heterocycle, cycloalkyl or hydroxyl.

The term “cycloalkyl” refers to cyclic alkyl groups of from 3 to 20 carbon atoms having a single cyclic ring or multiple condensed rings. Such cycloalkyl groups include, by way of example, single ring structures such as cyclopropyl, cyclobutyl, cyclopentyl, cyclooctyl, and the like, or multiple ring structures such as adamantanyl, and the like.

The cycloalkyl can optionally be substituted with one or more alkyl, alkenyl, alkoxy, halo, haloalkyl, hydroxy, hydroxyalkyl, aryl, heteroaryl, heterocycle, alkanoyl, alkoxycarbonyl, amino, imino, alkylamino, acylamino, nitro, trifluoromethyl, trifluoromethoxy, carboxy, carboxyalkyl, keto, thioxo, alkylthio, alkylsulfinyl, alkylsulfonyl, cyano, NR^{x}R^{y} and COOR^{x}, wherein each R^{x} and R^{y} are independently H, alkyl, aryl, heteroaryl, heterocycle, cycloalkyl or hydroxyl.

The cycloalkyl can optionally be at least partially unsaturated, thereby providing a cycloalkenyl.

The term “halo” refers to fluoro, chloro, bromo, and iodo. Similarly, the term “halogen” refers to fluorine, chlorine, bromine, and iodine.

“Haloalkyl” refers to alkyl as defined herein substituted by 1-4 halo groups as defined herein, which may be the same or different. Representative haloalkyl groups include, by way of example, trifluoromethyl, 3-fluorododecyl, 12,12,12-trifluorododecyl, 2-bromooctyl, 3-bromo-6-chloroheptyl, and the like.

The term “heteroaryl” is defined herein as a monocyclic, bicyclic, or tricyclic ring system containing one, two, or three aromatic rings and containing at least one nitrogen, oxygen, or sulfur atom in an aromatic ring, and which can be unsubstituted or substituted, for example, with one or more, and in particular one to three, substituents, like halo, alkyl, hydroxy, hydroxyalkyl, alkoxy, alkoxyalkyl, haloalkyl, nitro, amino, alkylamino, acylamino, alkylthio, alkylsulfinyl, and alkylsulfonyl. Examples of heteroaryl groups include, but are not limited to, 2H-pyrrolyl, 3H-indolyl, 4H-quinolizinyl, 4nH-carbazolyl, acridinyl, benzo[b]thienyl, benzothiazolyl, β-carbolinyl, carbazolyl, chromenyl, cinnaolinyl, dibenzo[b,d]furanyl, furazanyl, furyl, imidazolyl, imidizolyl, indazolyl, indolisinyl, indolyl, isobenzofuranyl, isoindolyl, isoquinolyl, isothiazolyl, isoxazolyl, naphthyridinyl, naptho[2,3-b], oxazolyl, perimidinyl, phenanthridinyl, phenanthrolinyl, phenarsazinyl, phenazinyl, phenothiazinyl, phenoxathiinyl, phenoxazinyl, phthalazinyl, pteridinyl, purinyl, pyranyl, pyrazinyl, pyrazolyl, pyridazinyl, pyridyl, pyrimidinyl, pyrimidinyl, pyrrolyl, quinazolinyl, quinolyl, quinoxalinyl, thiadiazolyl, thianthrenyl, thiazolyl, thienyl, triazolyl, and xanthenyl. In one embodiment the term “heteroaryl” denotes a monocyclic aromatic ring containing five or six ring atoms containing carbon and 1, 2, 3, or 4 heteroatoms independently selected from the group non-peroxide oxygen, sulfur, and N(Z) wherein Z is absent or is H, O, alkyl, phenyl or benzyl. In another embodiment heteroaryl denotes an ortho-fused bicyclic heterocycle of about eight to ten ring atoms derived therefrom, particularly a benz-derivative or one derived by fusing a propylene, or tetramethylene diradical thereto.

The heteroaryl can optionally be substituted with one or more alkyl, alkenyl, alkoxy, halo, haloalkyl, hydroxy, hydroxyalkyl, aryl, heterocycle, cycloalkyl, alkanoyl, alkoxycarbonyl, amino, imino, alkylamino, acylamino, nitro, trifluoromethyl, trifluoromethoxy, carboxy, carboxyalkyl, keto, thioxo, alkylthio, alkylsulfinyl, alkylsulfonyl, cyano, NR^{x}R^{y} and COOR^{x}, wherein each R^{x} and R^{y} are independently H, alkyl, aryl, heteroaryl, heterocycle, cycloalkyl or hydroxyl.

The term “heterocycle” refers to a saturated or partially unsaturated ring system, containing at least one heteroatom selected from the group oxygen, nitrogen, and sulfur, and optionally substituted with alkyl or C(═O)OR^{b}, wherein R^{b} is hydrogen or alkyl. Typically heterocycle is a monocyclic, bicyclic, or tricyclic group containing one or more heteroatoms selected from the group oxygen, nitrogen, and sulfur. A heterocycle group also can contain an oxo group (═O) attached to the ring. Non-limiting examples of heterocycle groups include 1,3-dihydrobenzofuran, 1,3-dioxolane, 1,4-dioxane, 1,4-dithiane, 2H-pyran, 2-pyrazoline, 4H-pyran, chromanyl, imidazolidinyl, imidazolinyl, indolinyl, isochromanyl, isoindolinyl, morpholine, piperazinyl, piperidine, piperidyl, pyrazolidine, pyrazolidinyl, pyrazolinyl, pyrrolidine, pyrroline, quinuclidine, and thiomorpholine.

The heterocycle can optionally be substituted with one or more alkyl, alkenyl, alkoxy, halo, haloalkyl, hydroxy, hydroxyalkyl, aryl, heteroaryl, cycloalkyl, alkanoyl, alkoxycarbonyl, amino, imino, alkylamino, acylamino, nitro, trifluoromethyl, trifluoromethoxy, carboxy, carboxyalkyl, keto, thioxo, alkylthio, alkylsulfinyl, alkylsulfonyl, cyano, NR^{x}R^{y} and COOR^{x}, wherein each R^{x} and R^{y} are independently H, alkyl, aryl, heteroaryl, heterocycle, cycloalkyl or hydroxyl.

Examples of nitrogen heterocycles and heteroaryls include, but are not limited to, pyrrole, imidazole, pyrazole, pyridine, pyrazine, pyrimidine, pyridazine, indolizine, isoindole, indole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, phenoxazine, phenothiazine, imidazolidine, imidazoline, piperidine, piperazine, indoline, morpholino, piperidinyl, tetrahydrofuranyl, and the like as well as N-alkoxy-nitrogen containing heterocycles. In one specific embodiment of the invention, the nitrogen heterocycle can be 3-methyl-5,6-dihydro-4H-pyrazino[3,2,1-jk]carbazol-3-ium iodide.

Another class of heterocyclics is known as “crown compounds” which refers to a specific class of heterocyclic compounds having one or more repeating units of the formula [—(CH₂—)ₐA-] where a is equal to or greater than 2, and A at each separate occurrence can be O, N, S or P. Examples of crown compounds include, by way of example only, [—(CH₂)₃—NH—]₃, [—((CH₂)₂—O)₄—((CH₂)₂—NH)₂] and the like. Typically such crown compounds can have from 4 to 10 heteroatoms and 8 to 40 carbon atoms.

The term “alkanoyl” refers to C(═O)R, wherein R is an alkyl group as previously defined.

The term “acyloxy” refers to —O—C(═O)R, wherein R is an alkyl group as previously defined. Examples of acyloxy groups include, but are not limited to, acetoxy, propanoyloxy, butanoyloxy, and pentanoyloxy. Any alkyl group as defined above can be used to form an acyloxy group.

The term “alkoxycarbonyl” refers to C(═O)OR, wherein R is an alkyl group as previously defined.

The term “amino” refers to —NH₂, and the term “alkylamino” refers to —NR₂, wherein at least one R is alkyl and the second R is alkyl or hydrogen. The term “acylamino” refers to RC(═O)N, wherein R is alkyl or aryl.

The term “imino” refers to —C═NH.

The term “nitro” refers to —NO₂.

The term “trifluoromethyl” refers to —CF₃.

The term “trifluoromethoxy” refers to —OCF₃.

The term “cyano” refers to —CN.

The term “hydroxy” or “hydroxyl” refers to —OH.

The term “oxy” refers to —O—.

The term “thio” refers to —S—.

The term “thioxo” refers to (═S).

The term “keto” refers to (═O).

The term “carbohydrate” refers to an essential structural component of living cells and source of energy for animals; includes simple sugars with small molecules as well as macromolecular substances; are classified according to the number of monosaccharide groups they contain. The term refers to one of a group of compounds including the sugars, starches, and gums, which contain six (or some multiple of six) carbon atoms, united with a variable number of hydrogen and oxygen atoms, but with the two latter always in proportion as to form water; as dextrose, {C₆H₁₂O₆}. The term refers to a compound or molecule that is composed of carbon, oxygen and hydrogen in the ratio of 2H:1C:10. Carbohydrates can be simple sugars such as sucrose and fructose or complex polysaccharide polymers such as chitin and starch.

The carbohydrate can optionally be substituted with one or more alkyl, alkenyl alkoxy, halo, haloalkyl, hydroxy, hydroxyalkyl, aryl, heteroaryl, heterocycle, cycloalkyl, alkanoyl, alkoxycarbonyl, amino, imino, alkylamino, acylamino, nitro, trifluoromethyl, trifluoromethoxy, carboxy, carboxyalkyl, keto, thioxo, alkylthio, alkylsulfinyl, alkylsulfonyl, cyano, NR^{x}R^{y} and/or COOR^{x}, wherein each R^{x} and R^{y} are independently H, alkyl, alkenyl, aryl, heteroaryl, heterocycle, cycloalkyl or hydroxy.

The sugar can be a monosaccharide, disaccharide, oligosaccharide, or polysaccharide. The sugar can have a beta (β) or alpha (α) stereochemistry, can have an (R) or (S) relative configuration, can exist as the (+) or (−) isomer, and can exist in the D or L configuration. For example, the sugar can be β-D-glucose.

The term “saccharide” refers to any sugar or other carbohydrate, especially a simple sugar or carbohydrate. Saccharides are an essential structural component of living cells and source of energy for animals. The term includes simple sugars with small molecules as well as macromolecular substances. Saccharides are classified according to the number of monosaccharide groups they contain.

The term “polysaccharide” refers to a type of carbohydrate that contains sugar molecules that are linked together chemically, i.e., through a glycosidic linkage. The term refers to any of a class of carbohydrates whose are carbohydrates that are made up of chains of simple sugars. Polysaccharides are polymers composed of multiple units of monosaccharide (simple sugar).

The term “oligosaccharide” refers to compounds containing two to ten monosaccharide units.

Suitable exemplary sugars include, e.g., ribose, glucose, fructose, mannose, idose, gulose, galactose, altrose, allose, xylose, arabinose, threose, glyceraldehydes, and erythrose.

As used herein, “starch” refers to the complex polysaccharides present in plants, consisting of a-(1,4)-D-glucose repeating subunits and α-(1,6)-glucosidic linkages.

As used herein, “dextrin” refers to a polymer of glucose with intermediate chain length produced by partial degradation of starch by heat, acid, enzyme, or a combination thereof.

As used herein, “maltodextrin” or “glucose polymer” refers to non-sweet, nutritive saccharide polymer that consists of D-glucose units linked primarily by α,-1,4 bonds and that has a DE (dextrose equivalent) of less than 20. See, e.g., The United States Food and Drug Administration (21 C.F.R. paragraph 184.1444). Maltodextrins are partially hydrolyzed starch products. Starch hydrolysis products are commonly characterized by their degree of hydrolysis, expressed as dextrose equivalent (DE), which is the percentage of reducing sugar calculated as dextrose on dry-weight basis.

As to any of the above groups, which contain one or more substituents, it is understood, of course, that such groups do not contain any substitution or substitution patterns which are sterically impractical and/or synthetically non-feasible. In addition, the compounds of this invention include all stereochemical isomers arising from the substitution of these compounds.

Selected substituents within the compounds described herein are present to a recursive degree. In this context, “recursive substituent” means that a substituent may recite another instance of itself. Because of the recursive nature of such substituents, theoretically, a large number may be present in any given claim. One of ordinary skill in the art of medicinal chemistry understands that the total number of such substituents is reasonably limited by the desired properties of the compound intended. Such properties include, by of example and not limitation, physical properties such as molecular weight, solubility or log P, application properties such as activity against the intended target, and practical properties such as ease of synthesis.

Recursive substituents are an intended aspect of the invention. One of ordinary skill in the art of medicinal and organic chemistry understands the versatility of such substituents. To the degree that recursive substituents are present in an claim of the invention, the total number will be determined as set forth above.

The compounds described herein can be administered as the parent compound, a pro-drug of the parent compound, or an active metabolite of the parent compound.

“Pro-drugs” are intended to include any covalently bonded substances which release the active parent drug or other formulas or compounds of the present invention in vivo when such pro-drug is administered to a mammalian subject. Pro-drugs of a compound of the present invention are prepared by modifying functional groups present in the compound in such a way that the modifications are cleaved, either in routine manipulation in vivo, to the parent compound. Pro-drugs include compounds of the present invention wherein the carbonyl, carboxylic acid, hydroxy or amino group is bonded to any group that, when the pro-drug is administered to a mammalian subject, cleaves to form a free carbonyl, carboxylic acid, hydroxy or amino group. Examples of pro-drugs include, but are not limited to, acetate, formate and benzoate derivatives of alcohol and amine functional groups in the compounds of the present invention, and the like.

“Metabolite” refers to any substance resulting from biochemical processes by which living cells interact with the active parent drug or other formulas or compounds of the present invention in vivo, when such active parent drug or other formulas or compounds of the present are administered to a mammalian subject. Metabolites include products or intermediates from any metabolic pathway.

“Metabolic pathway” refers to a sequence of enzyme-mediated reactions that transform one compound to another and provide intermediates and energy for cellular functions. The metabolic pathway can be linear or cyclic. Obviously, numerous modifications and variations of the present invention are possible in light of the above teachings. It is therefore to be understood that within the scope of the appended claims, the invention may be practiced otherwise than as specifically described herein.

The practice of the present invention will employ, unless otherwise indicated, conventional techniques of molecular biology, virology, microbiology, recombinant DNA, and immunology, which are within the skill of the art.

### Reactive Oxygen Species and NADPH Oxidase

In general, vertebrates possess two fundamental mechanisms to respond to infection, the innate and the acquired immune system (Fearon et al., 1996). Innate, or natural immunity is the ability to respond immediately to an infectious challenge, regardless of previous exposure of the host to the invading agent. Elements of the innate system include phagocytic cells, namely polymorphonuclear leukocytes (PMNs) and mononuclear phagocytes (e.g., macrophages), and the complement cascade of circulating soluble preenzymic proteins. These elements constitute a relatively nonspecific ‘pattern recognition’ system which has functional analogues in the immune system of a wide variety of multicellular organisms, including plants (Enyedi et al., 1992) and insects (Hoffmann et al., 1999). As such, these evolutionary ancient elements represent a rapid and sensitive surveillance mechanism of host defense when the organism is challenged with an invading microorganism previously ‘unseen’ by the host's immune system. In contrast to the innate system, adaptive immunity is restricted to vertebrates and represents a precisely tuned system by which host cells define specifically the nature of the invading pathogen or tumor cell (Janeway et al., 1994). Such precision, however, requires time for antigens to be processed and specific lymphocytes and antibodies to be generated. Therefore, the adaptive system is slower to respond to new challenges than is the innate system which lacks specificity (Fearon et al., 1996).

Granulocytes arise from pluripotent stem cells located in the bone marrow, and include eosinophils, basophils, and neutrophils. PMNs are the most numerous leukocytes in the human peripheral circulation, and take their name from their typically multilobed nucleus. The daily production of mature PMNs in a healthy adult is in the order of 10¹¹ cells. During acute infection or other inflammatory stresses, PMNs are mobilized from the marrow reservoir, containing up to 10 times the normal daily neutrophil requirement (Nauseef et al., 2000). PMNs are motile, and very plastic cells which allows them to move to sites of inflammation where they serve as a first line of defense against infectious microorganisms. For this purpose, PMNs contain granules filled with proteolytic and other cytotoxic enzymes (Schettler et al., 1991; Borregaard et al., 1997). Besides releasing enzymes, PMNs are also able to phagocytose and to convert oxygen into highly reactive oxygen species (ROS). Following phagocytosis, ingested microorganisms may be killed inside the phagosome by a combined action of enzyme activity and ROS production.

Upon activation, PMNs start to consume a vast amount of oxygen which is converted into ROS, a process known as the respiratory or oxidative burst (Babior et al., 1976; Babior et al., 1978). This process is dependent on the activity of the enzyme NADPH oxidase. This oxidase can be activated by both receptor-mediated and receptor-independent processes. Typical receptor-dependent stimuli are complement components C5a, C3b and iC3b (Ogle et al., 1988), the bacterium-derived chemotactic tripeptide N-formyl-Met-Leu-Phe (fMLP) (Williams et al., 1977), the lectin concanavalin A (Weinbaum et al., 1980), and opsonized zymosan (OPZ) (Whitin et al., 1985). Receptor-independent stimuli include long-chain unsaturated fatty acids and phorbol 12-myristate 13-acetate (PMA) (Schnitzler et al., 1997). Upon activation, the oxidase accepts electrons from NADPH at the cytosolic side of the membrane and donates these to molecular oxygen at the other side of the membrane, either at the outside of the cells or in the phagosomes containing ingested microorganisms. In this way, a one-electron reduction of oxygen to superoxide anion (.O₂—) is catalyzed at the expense of NADPH as depicted in the following equation:

2O₂+NADPH→2.O₂—+NADP⁺+H⁺

Most of the oxygen consumed in this way will not be present as .O₂—, but can be accounted for as hydrogen peroxide which is formed from dismutation of the superoxide radical (Hampton, 1998; Roos et al., 1984):

.O₂+e−+H⁺→H₂O₂

However, hydrogen peroxide (H₂O₂) is bactericidal only at high concentrations (Hyslop et al., 1995) while exogenously generated superoxide does not kill bacteria directly (Babior et al., 1975; Rosen et al., 1979) because of its limited membrane permeability. Therefore, a variety of secondary oxidants have been proposed to account for the destructive capacity of PMNs.

Hydroxyl radicals (.OH), formed by the iron catalyzed Fenton reaction, are extremely reactive with most biological molecules although they have a limited range of action (Samuni et al., 1988).

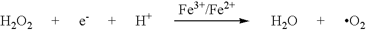

Singlet oxygen (¹O₂) is often seen as the electronically excited state of oxygen and may react with membrane lipids initiating peroxidation (Halliwell, 1978). Most of the H₂O₂ generated by PMNs is consumed by myeloperoxidase (MPO), an enzyme released by stimulated PMNs (Kettle et al., 1997; Nauseef, 1988; Zipfel et al., 1997; Klebanoff, 1999). This heme-containing peroxidase is a major constituent of azurophilic granules and is unique in using H₂O₂ to oxidize chloride ions to the strong non-radical oxidant hypochlorous acid (HOCl) (Harrison et al., 1976). Other substrates of MPO include iodide, bromide, thiocyanite, and nitrite (Van Dalen et al., 1997; Vliet et al., 1997).

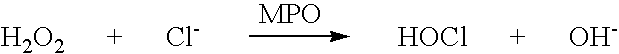

HOCl is the most bactericidal oxidant known to be produced by the PMN (Klebanoff, 1968), and many species of bacteria are killed readily by the MPO/H₂O₂/chloride system (Albrich et al., 1982).

In experimental settings, ROS production by activated phagocytes can be detected using enhancers such as luminol or lucigenin (Faulkner et al., 1993). For ROS-detection, lucigenin must first undergo reduction, while luminol must undergo one-electron oxidation to generate an unstable endoperoxide, the decomposition of which generates light by photon-emission (Halliwell et al., 1998). Luminol largely detects HOCl, which means that luminol detection is mainly dependent on the MPO/H₂O₂ system (McNally et al., 1996), while detection using lucigenin is MPO-independent and more specific for .O₂— (Anniansson et al., 1984). Luminol is able to enter the cell and thereby detects intra- as well as extracellularly produced ROS (Dahlgren et al., 1989), while lucigenin is practically incapable of passing the cell membrane and thereby only detects extracellular events (Dahlgren et al., 1985). However, results should be interpreted with care, because real specificity can never be assumed with any of these light-emission-enhancing compounds (Liochev et al., 1997).

Production of .O₂— seems to occur within all aerobic cells, to an extent dependent on O₂ concentration. In mitochondria, 1-3% of electrons are thought to form .O₂—. The fact that ROS are also quantitatively significant products of aerobic metabolism is illustrated by the following calculation: a normal adult (assuming 70 kg body weight) at rest utilizes 3.5 mL O₂/kg/min, which is identical to 352.8 l/day or 14.7 mol/day. If 1% makes .O₂— this gives 0.147 mol/day or 53.66 mol/year or about 1.7 kg of .O₂— per year. During the respiratory burst, the increase in O₂ uptake can be 10 to 20 times that of the resting O₂ consumption of neutrophils (Halliwell et al., 1998).

The NADPH oxidase, responsible for ROS production, is a multi-component enzyme system which is unassembled (and thereby inactive) in resting PMNs. However, activation of the phagocyte, e.g., by the binding of opsonized microorganisms to cell-surface receptors, leads to the assembly of an active enzyme complex on the plasma membrane (Clark, 1990; Segal et al., 1993). The critical importance of a functioning NADPH oxidase in normal host defense is most dramatically illustrated by the recurrent bacterial and fungal infections observed in individuals with chronic granulomatous disease (CGD), a disorder in which the oxidase is non-functional due to a deficiency in one of the constituting protein components (Smith et al., 1991; Dinauer et al., 1993; Dinauer et al., 1987; Volpp et al., 1988). PMNs from such patients, lacking a functionally competent oxidase, fail to generate .O₂— upon stimulation. Although the formation of ROS by stimulated PMNs may be a physiological response which is advantageous to the host, it can also be detrimental in many inflammatory states in which these radicals might give rise to excessive tissue damage (Weiss, 1989; Fantone et al., 1985; Jackson et al., 1988).

Essential components of the NADPH oxidase include plasma membrane and cytosolic proteins. The key plasma membrane component is a heterodimeric flavocytochrome b which is composed of a 91-kDa glycoprotein (gp91^{phox}) and a 22-kDa protein (p22^{phox}) (Rotrosen et al., 1992; Segel et al., 1992). Flavocytochrome b serves to transfer electrons from NADPH to molecular oxygen, resulting in the generation of O₂—. In PMN membranes, a low-molecular-weight GTP-binding protein, Rap1A, is associated with flavocytochrome b and plays an important role in NADPH oxidase regulation in vivo (Quinn et al., 1989; Gabig et al., 1995). Furthermore, cytosolic proteins p47^{phox}, p67^{phox}, and a second low-molecular-weight GTP-binding protein, Rac2 are required for NADPH oxidase activity (Volpp et al., 1988; Lomax et al., 1989a; Lomax et al., 1989b) and these three proteins associate with flavocytochrome b to form the functional NADPH oxidase (Clark et al., 1990; Heyworth et al., 1991; Quinn et al., 1993; DeLeo et al., 1996). Additionally, a cytosolic protein, p40^{phox}, has been identified, but its role in oxidase function is not completely defined (Wientjes et al., 1993). According to the current model of NADPH oxidase assembly, p47^{phox} and p67^{phox} translocate en bloc to associate with flavocytochrome b during PMN activation (DeLeo et al., 1996; Park et al., 1992; Iyer et al., 1994). Rac2 translocates simultaneously, but independently of the other two cytosolic components, to associate with the membrane-bound flavocytochrome b (Heyworth et al., 1994; Dorseuil et al., 1995). Studies of oxidase assembly in PMNs of patients with various forms of CGD suggest that p47phox binds directly to flavocytochrome b (Heyworth et al., 1991) and at least six regions of flavocytochrome b have been identified as putative sites for interaction with p47^{phox}, including four sites on gp91^{phox} and two sites on p22^{phox} (Kleinberg et al., 1990; Leusen et al., 1994; Leto et al, 1994; Leusen et al., 1994; Nakanish et al., 1992; DeLeo et al., 1995; Sumimoto et al., 1994; Finan et al., 1994).

### Methods of the Invention

The methods of the invention include methods to identify agents that alter virus transduction for viruses with redox sensitive intracellular pathways and the use of those agents to enhance or inhibit viral transduction, methods to modify viral capsids to alter intracellular viral redox sensitivity and modified viruses produced by the methods, and methods to identify receptor and co-receptors for viruses that traffic through Rac containing endosomes. Viruses useful in the methods of the invention are those that are redox sensitive or may be modified to be more or less redox sensitive, e.g., viruses having pathways that include association with Rac1 containing endosomes or alter NADPH oxidase activity, e.g., adenovirus, poxviruses, lentivirus, hepatitis viruses, parvovirus, coxsackievirus and/or influenza viruses. For example, viruses that are redox sensitive may be screened with one or more agents to detect agents that increase or decrease viral transduction by increasing or decreasing NADPH oxidase activity. Viruses may be modified, for instance, viral capsids of redox sensitive or insensitive viruses may be modified and those viruses screened with one or more agents to detect agents that increase or decrease viral transduction.

### Uses of Viruses for Gene Transfer

Viral vectors can be used for administration to an individual for purposes of gene therapy or vaccination. Suitable diseases for therapy include but are not limited to those induced by viral, bacterial, or parasitic infections, various malignancies and hyperproliferative conditions, autoimmune conditions, and congenital deficiencies.

Gene therapy can be conducted to enhance the level of expression of a particular protein either within or secreted by the cell. Vectors of this invention may be used to genetically alter cells either for gene marking, replacement of a missing or defective gene, or insertion of a therapeutic gene. Alternatively, a polynucleotide may be provided to the cell that decreases the level of expression. This may be used for the suppression of an undesirable phenotype, such as the product of a gene amplified or overexpressed during the course of a malignancy, or a gene introduced or overexpressed during the course of a microbial infection. Expression levels may be decreased by supplying a therapeutic or prophylactic polynucleotide comprising a sequence capable, for example, of forming a stable hybrid with either the target gene or RNA transcript (antisense therapy), capable of acting as a ribozyme to cleave the relevant mRNA or capable of acting as a decoy for a product of the target gene.

The introduction of viral vectors by the methods of the present invention may involve use of any number of delivery techniques (both surgical and non-surgical) which are available and well known in the art. Such delivery techniques, for example, include vascular catheterization, cannulization, injection, inhalation, endotracheal, subcutaneous, inunction, topical, oral, percutaneous, intra-arterial, intravenous, and/or intraperitoneal administrations. Vectors can also be introduced by way of bioprostheses, including, by way of illustration, vascular grafts (PTFE and dacron), heart valves, intravascular stents, intravascular paving as well as other non-vascular prostheses. General techniques regarding delivery, frequency, composition and dosage ranges of vector solutions are within the skill of the art.

In particular, for delivery of a vector of the invention to a tissue, any physical or biological method that will introduce the vector to a host animal can be employed. Vector means both a bare recombinant vector and vector DNA packaged into viral coat proteins, as is well known for parvovirus administration. Simply dissolving a viral vector in phosphate buffered saline has been demonstrated to be sufficient to provide a vehicle useful for muscle tissue expression, and there are no known restrictions on the carriers or other components that can be coadministered with the vector (although compositions that degrade DNA should be avoided in the normal manner with vectors). Pharmaceutical compositions can be prepared as injectable formulations or as topical formulations to be delivered to the muscles by transdermal transport. Numerous formulations for both intramuscular injection and transdermal transport have been previously developed and can be used in the practice of the invention. The vectors can be used with any pharmaceutically acceptable carrier for ease of administration and handling.

For purposes of intramuscular injection, solutions in an adjuvant such as sesame or peanut oil or in aqueous propylene glycol can be employed, as well as sterile aqueous solutions. Such aqueous solutions can be buffered, if desired, and the liquid diluent first rendered isotonic with saline or glucose. Solutions of the viral vector as a free acid (DNA contains acidic phosphate groups) or a pharmacologically acceptable salt can be prepared in water suitably mixed with a surfactant such as hydroxypropylcellulose. A dispersion of viral particles can also be prepared in glycerol, liquid polyethylene glycols and mixtures thereof and in oils. Under ordinary conditions of storage and use, these preparations contain a preservative to prevent the growth of microorganisms. In this connection, the sterile aqueous media employed are all readily obtainable by standard techniques well-known to those skilled in the art.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, liquid polyethylene glycol and the like), suitable mixtures thereof, and vegetable oils. The proper fluidity can be maintained, for example, by the use of a coating such as lecithin, by the maintenance of the required particle size in the case of a dispersion and by the use of surfactants. The prevention of the action of microorganisms can be brought about by various antibacterial and antifungal agents, for example, parabens, chlorobutanol, phenol, sorbic acid, thimerosal and the like. In many cases it will be preferable to include isotonic agents, for example, sugars or sodium chloride. Prolonged absorption of the injectable compositions can be brought about by use of agents delaying absorption, for example, aluminum monostearate and gelatin.

Sterile injectable solutions are prepared by incorporating the AAV vector in the required amount in the appropriate solvent with various of the other ingredients enumerated above, as required, followed by filtered sterilization. Generally, dispersions are prepared by incorporating the sterilized active ingredient into a sterile vehicle which contains the basic dispersion medium and the required other ingredients from those enumerated above. In the case of sterile powders for the preparation of sterile injectable solutions, the preferred methods of preparation are vacuum drying and the freeze drying technique which yield a powder of the active ingredient plus any additional desired ingredient from the previously sterile-filtered solution thereof.

For purposes of topical administration, dilute sterile, aqueous solutions (usually in about 0.1% to 5% concentration), otherwise similar to the above parenteral solutions, are prepared in containers suitable for incorporation into a transdermal patch, and can include known carriers, such as pharmaceutical grade dimethylsulfoxide (DMSO).

Compositions of this invention may be used in vivo as well as ex vivo. In vivo gene therapy comprises administering the vectors of this invention directly to a subject. Pharmaceutical compositions can be supplied as liquid solutions or suspensions, as emulsions, or as solid forms suitable for dissolution or suspension in liquid prior to use. For administration into the respiratory tract, a preferred mode of administration is by aerosol, using a composition that provides either a solid or liquid aerosol when used with an appropriate aerosolubilizer device. Another preferred mode of administration into the respiratory tract is using a flexible fiberoptic bronchoscope to instill the vectors. Typically, the viral vectors are in a pharmaceutically suitable pyrogen-free buffer such as Ringer's balanced salt solution (pH 7.4). Although not required, pharmaceutical compositions may optionally be supplied in unit dosage form suitable for administration of a precise amount.

An effective amount of virus is administered, depending on the objectives of treatment. An effective amount may be given in single or divided doses. Where a low percentage of transduction can cure a genetic deficiency, then the objective of treatment is generally to meet or exceed this level of transduction. In some instances, this level of transduction can be achieved by transduction of only about 1 to 5% of the target cells, but is more typically 20% of the cells of the desired tissue type, usually at least about 50%, preferably at least about 80%, more preferably at least about 95%, and even more preferably at least about 99% of the cells of the desired tissue type. As a guide, the number of vector particles present in a single dose given by bronchoscopy will generally be at least about 1×10⁸, and is more typically 5×10⁸, 1×10¹⁰, and on some occasions 1×10¹¹ particles, including both DNAse-resistant and DNAse-susceptible particles. In terms of DNAse-resistant particles, the dose will generally be between 1×10⁶ and 1×10¹⁴ particles, more generally between about 1×10⁸ and 1×10¹² particles. The treatment can be repeated as often as every two or three weeks, as required, although treatment once in 180 days may be sufficient.

To confirm the presence of the desired DNA sequence in the host cell, a variety of assays may be performed. Such assays include, for example, “molecular biological” assays well known to those of skill in the art, such as Southern and Northern blotting, RT-PCR and PCR; “biochemical” assays, such as detecting the presence of a polypeptide expressed from a gene present in the vector, e.g., by immunological means (immunoprecipitations, immunoaffinity columns, ELISAs and Western blots) or by any other assay useful to identify the presence and/or expression of a particular nucleic acid molecule falling within the scope of the invention.

To detect and quantitate RNA produced from introduced DNA segments, RT-PCR may be employed. In this application of PCR, it is first necessary to reverse transcribe RNA into DNA, using enzymes such as reverse transcriptase, and then through the use of conventional PCR techniques amplify the DNA. In most instances PCR techniques, while useful, will not demonstrate integrity of the RNA product. Further information about the nature of the RNA product may be obtained by Northern blotting. This technique demonstrates the presence of an RNA species and gives information about the integrity of that RNA. The presence or absence of an RNA species can also be determined using dot or slot blot Northern hybridizations. These techniques are modifications of Northern blotting and only demonstrate the presence or absence of an RNA species.

While Southern blotting and PCR may be used to detect the DNA segment in question, they do not provide information as to whether the DNA segment is being expressed. Expression may be evaluated by specifically identifying the polypeptide products of the introduced DNA sequences or evaluating the phenotypic changes brought about by the expression of the introduced DNA segment in the host cell.

Thus, the effectiveness of the genetic alteration can be monitored by several criteria, including analysis of physiological fluid samples, e.g., urine, plasma, serum, blood, cerebrospinal fluid or nasal or lung washes. Samples removed by biopsy or surgical excision may be analyzed by in situ hybridization, PCR amplification using vector-specific probes, RNAse protection, immunohistology, or immunofluorescent cell counting. When the vector is administered by bronchoscopy, lung function tests may be performed, and bronchial lavage may be assessed for the presence of inflammatory cytokines. The treated subject may also be monitored for clinical features, and to determine whether the cells express the function intended to be conveyed by the therapeutic or prophylactic polynucleotide.

The decision of whether to use in vivo or ex vivo therapy, and the selection of a particular composition, dose, and route of administration will depend on a number of different factors, including but not limited to features of the condition and the subject being treated. The assessment of such features and the design of an appropriate therapeutic or prophylactic regimen is ultimately the responsibility of the prescribing physician.

### Exemplary Compounds Useful in the Methods of the Invention

Agents that may be useful in the methods of the invention include but are not limited to interleukins, anaphylatoxins, angiotensin II, NSAIDs, e.g., diclofenac, cathelicidins, proline rich antimicrobial peptides, C reactive protein, haemozoin, iodolactones or iodoaldehydes, e.g., iodohexadecanal, carotenoids, ACE inhibitors, antihypertensive drugs, steroids, methotrexate, antibiotics such as tetracycline, nitroarenes, quinines, aromatic N-oxides, aspirin, flavonoids, allicin, αtocopherol, quercetin, catechins, isothiocyanates, NAC, beta carotene, genistein, daidzein, propylgallate, curcumin, pyridoxine-pyrrolidone carboxylates, PDE inhibitors, class IV anesthetics, volatile anethestics, hypocholesterolemic drugs, cyclosporine A, polyphenols, long chain omega 6 arachidonic acid, metadoxine, tirapazamine, AQ4N, RSR13, molexafin Gd, HIF1 inhibitors, nitric oxide donors, nitroaspirin, eicosanoids, corticosteroids, auranofin, butyrate, propionate, oxyresveratrol, reserverol, thiopental succinylcholine, dicoumerol, triptolide, agents disclosed in U.S. Pat. Nos. 6,927,238, 6,864,288, 6,713,605, 6,184,203, 6,090,851, 5,902,831, 5,763,496, 5,726,155, 5,244,916, 5,118,601, and 6,172,116, U.S. published application 20040120926, and U.S. published application 20040001818, cationic peptides such as PR-39, a proline rich antibacterial peptide, DPI, cromolyn, NOS oxidase inhibitors, phenyl arsine oxide, histamine, inhibitors of PLD activity, TNF-alpha, TGF-beta, IL-1, interferon, PDGF, and EGF, Rac, formyl peptides, PMA, calcium ionophores, e.g., ionomycin, or agmatine.

### Exemplary Nox Activators

Agents that may be useful to enhance Nox activity may include but are not limited to interleukins, phospholipids, anaphylatoxins, angiotensin II, angiopoietin, VEGF, streptozotocin, BMP4, gp 91ds-tat (see Walch et al., Athero., 167:285 (2006), Wy-14643 (Reisyn et al., Can. Res., 60:4798 (2000)), formyl peptides such as f-met-Leu-Phe, BDNF (U.S. application publication No. 20060135600), NSAIDs, e.g., diclofenac, TNF-alpha, TGF-beta, IL-1, interferon, PDGF, EGF, Rac, PMA, calcium ionophores, e.g., ionomycin, agmatine, and those disclosed in U.S. Pat. No. 6,172,116.

### Exemplary Nox Inhibitors

Agents that may be useful to inhibit Nox activity include but are not limited to those disclosed in U.S. Pat. Nos. 7,202,053, 7,202,030, 7,067,158, 6,927,238, 6,864,288, 6,713,605, 6,184,203, 6,090,851, 5,902,831, 5,763,496, 5,726,155, 5,244,916, and 5,118,601, U.S. published applications 20040120926, 20060160095, 20060089362, and 20040001818, cationic peptides such as PR-39, a proline rich antibacterial peptide, DPI, piroxicam, MnTMPₓp (Franco et al., Life Sci., 80:709 (2007)), 1NAME (Coyle et al., ASAIO J., 2007), azelnidipine, atorvastatin, parabutoporin, NAC, staurosporine, diisopropylfluorophosphonate, catechols, e.g., methyl substituted catechols, 4-(2-aminoethyl)-benzensulfonyl fluoride, stilbene type phyto-alexin reservatrol, aminoguanidine, ON0174, S17834 (benzo[b]pyran-4-one), suramin, sulphonated aryl or benzamide derivatives (U.S. published application No. 20070037883), isoprenylation inhibitors such as lovastatin and compactin, benzofuranyl and benzothienyl thioalkanes, cromolyn, NOS oxidase inhibitors, phenyl arsine oxide, histamine, inhibitors of PLD activity, and a compound of formula (I).

Compounds of formula (I) are suitable potent and selective inhibitors of NADPH oxidase:

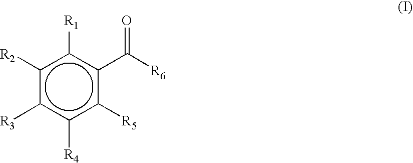

wherein,

R¹ is H, alkyl, alkenyl, alkoxy, halo, haloalkyl, hydroxy, hydroxyalkyl, aryl, heteroaryl, heterocycle, cycloalkyl, alkanoyl, alkoxycarbonyl, amino, imino, alkylamino, acylamino, nitro, trifluoromethyl, trifluoromethoxy, carboxy, carboxyalkyl, alkylthio, alkylsulfinyl, alkylsulfonyl, cyano, NR^{x}R^{y} or COOR^{x}, wherein each R^{x} and R^{y} is independently H, alkyl, alkenyl, aryl, heteroaryl, heterocycle, cycloalkyl or hydroxy;

R² is H, alkyl, alkenyl, alkoxy, halo, haloalkyl, hydroxy, hydroxyalkyl, aryl, heteroaryl, heterocycle, cycloalkyl, alkanoyl, alkoxycarbonyl, amino, imino, alkylamino, acylamino, nitro, trifluoromethyl, trifluoromethoxy, carboxy, carboxyalkyl, alkylthio, alkylsulfinyl, alkylsulfonyl, cyano, NR^{x}R^{y} or COOR^{x}, wherein each R^{x} and R^{y} is independently H, alkyl, alkenyl, aryl, heteroaryl, heterocycle, cycloalkyl or hydroxy;

R³ is H, alkyl, alkenyl, alkoxy, halo, haloalkyl, hydroxy, hydroxyalkyl, aryl, heteroaryl, heterocycle, cycloalkyl, alkanoyl, alkoxycarbonyl, amino, imino, alkylamino, acylamino, nitro, trifluoromethyl, trifluoromethoxy, carboxy, carboxyalkyl, alkylthio, alkylsulfinyl, alkylsulfonyl, cyano, O—R^{z}, NR^{x}R^{y} or COOR^{x}, wherein each R^{x} and R^{y} is independently H, alkyl, alkenyl, aryl, heteroaryl, heterocycle, cycloalkyl or hydroxy; and wherein R^{z} is a monovalent radical of a carbohydrate.

R⁴ is H, alkyl, alkenyl, alkoxy, halo, haloalkyl, hydroxy, hydroxyalkyl, aryl, heteroaryl, heterocycle, cycloalkyl, alkanoyl, alkoxycarbonyl, amino, imino, alkylamino, acylamino, nitro, trifluoromethyl, trifluoromethoxy, carboxy, carboxyalkyl, alkylthio, alkylsulfinyl, alkylsulfonyl, cyano, NR^{x}R^{y} or COOR^{x}, wherein each R^{x} and R^{y} is independently H, alkyl, alkenyl, aryl, heteroaryl, heterocycle, cycloalkyl or hydroxy;

R⁵ is H, alkyl, alkenyl, alkoxy, halo, haloalkyl, hydroxy, hydroxyalkyl, aryl, heteroaryl, heterocycle, cycloalkyl, alkanoyl, alkoxycarbonyl, amino, imino, alkylamino, acylamino, nitro, trifluoromethyl, trifluoromethoxy, carboxy, carboxyalkyl, alkylthio, alkylsulfinyl, alkylsulfonyl, cyano, NR^{x}R^{y} or COOR^{x}, wherein each R^{x} and R^{y} is independently H, alkyl, alkenyl, aryl, heteroaryl, heterocycle, cycloalkyl or hydroxy; and

R⁶ is H, alkyl, alkoxy, haloalkyl, hydroxy, hydroxyalkyl, aryl, heteroaryl, heterocycle, cycloalkyl, amino, alkylamino, acylamino, or NR^{x}R^{y}, wherein R^{x} and R^{y} are each independently H, alkyl, alkenyl, aryl, heteroaryl, heterocycle, cycloalkyl or hydroxy;

or a pharmaceutically acceptable salt thereof.

Compounds of formula (Ia) are suitable potent and selective inhibitor of NADPH oxidase:

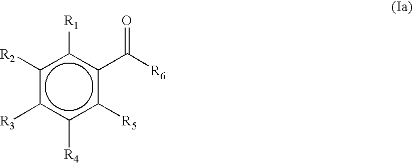

wherein,

R¹ is H;

R² is alkoxy;

R³ is hydroxyl, alkoxy or O—R^{z}, wherein R^{z} is a monovalent radical of a carbohydrate;

R⁴ is H, alkoxy or alkyl;

R⁵ is H or hydroxyl; and

R⁶ is alkyl, haloalkyl, hydroxyalkyl, aryl, heteroaryl, heterocycle, cycloalkyl, amino, alkylamino, or NR^{x}R^{y}, wherein R^{x} and R^{y} are each independently H, alkyl, alkenyl, aryl, heteroaryl, heterocycle, cycloalkyl or hydroxy;

or a pharmaceutically acceptable salt thereof.

Compounds of formula (Ib) are suitable potent and selective inhibitor of NADPH oxidase:

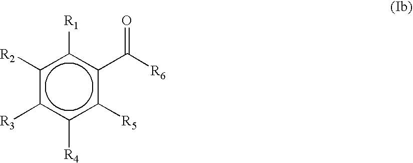

wherein,

R¹ is H;

R² is alkoxy;

R³ is hydroxyl, alkoxy O—R^{z}, wherein R^{z} is a monovalent radical of a carbohydrate;

R⁴ is H, alkyl or alkoxy;

R⁵ is H or hydroxyl; and

R⁶ is alkyl;

or a pharmaceutically acceptable salt thereof.

### Specific Ranges and Values:

Regarding the compound of formula (I): a specific value for R¹ is H; a specific value for R² is alkoxy; another specific value for R² is methoxy; a specific value for R³ is hydroxyl; another specific value for R³ is alkoxy substituted with hydroxyl; another specific value for R³ is 2-hydroxyl-ethoxy; another specific value for R³ is hydroxyl, a specific value for R⁴ is H; another specific value for R⁴ is alkoxy; another specific value for R⁴ is methoxy; another specific value for R⁴ is alkyl; another specific value for R⁴ is methyl; a specific value for R⁵ is H; another specific value for R⁵ is hydroxyl; a specific value for R⁶ is alkyl; and another specific value for R⁶ is methyl.

Regarding the compound of formula (Ia), a specific value for R² is alkoxy. Another specific value for R is methoxy. A specific value for R⁶ is alkyl. Another specific value for R is methyl.

Regarding the compound of formula (Ib), a specific value for R² is alkoxy. Another specific value for R² is methoxy. A specific value for R⁶ is methyl.

A specific compound of formulas (I), (Ia) and (Ib) is apocynin. Apocynin (4-Hydroxy-3-methoxyacetophenone; acetovanillone; a compound of formula II), a cell-permeable phenol, is a potent and selective inhibitor of NADPH oxidase.

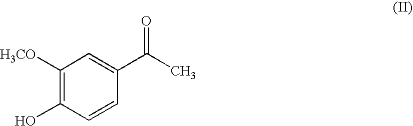

Apocynin is found in dry rhizomes and roots of Picrorhiza species, for example P. kurrooa and P. scrophulariiflora; the latter is also known as Neopicrorhiza scrophulariiflora. Apocynin may also be obtained from other sources, e.g., from the rhizome of Canadian hemp (Apocymum cannabinum) or other Apocynum species (e.g., A. androsaemifolium) or from the rhizomes of Iris species, provided that the extracts do not contain substantial amounts of cardiac glycosides. Picrorhiza kurroa Royle ex Benth is a perennial woody herb, and a crude extract there includes apocynin.

A Picrorhiza extract can be obtained by extracting the rhizomes of Picrorhiza species and subjecting the extract to column chromatography. Alternatively, extracts with high amounts of phenolic compounds can be obtained by pretreating the plant material with mineral acid to convert glycosides to their respective aglycones. If desired, the material may then be defatted to remove wax and other highly lipophilic matter. The material is extracted, for example with ethyl acetate and/or ethanol. The organic solvent is removed and an aqueous solution is obtained. The pH of the extract is increased to 10, e.g., with sodium hydroxide, to deprotonate phenolic compounds and to retain them in the aqueous phase. The aqueous solution is then washed, e.g., with diethyl ether to remove cucurbitacins. The aqueous phase is then reacidified to neutralise phenolic compounds and again extracted with, e.g., diethyl ether. The organic phase is collected and the solvent removed.

Additional suitable compounds of formula (I) include, e.g., compounds of the formula:

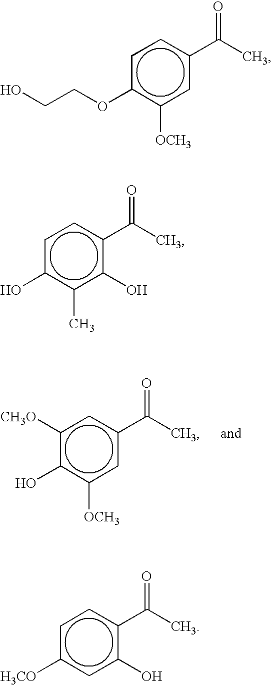

Other compounds useful in therapeutic or prophylactic methods to inhibit or prevent ROS include, but are not limited, to antioxidants in general, azelnidipine or other calcium antagonists, olmesartan or other AT1 receptor blockers, glucocorticoids, e.g., dexamethazone or hydrocortisone, beta-adrenergic agonists, e.g., isoproterenol, lipocortin, pyridine, polyphenols, e.g., vanillin, 4-nitroguaiacol, folic acid and metabolic antagonists thereof, and imidazoles, as well as RNAi, or combinations thereof.

### Dosages, Formulations and Routes of Administration of the Agents of the Invention

The agents of the invention can be formulated as pharmaceutical compositions and administered to a mammalian host, such as a human patient in a variety of forms adapted to the chosen route of administration, i.e., orally or parenterally, by intravenous, intramuscular, topical or subcutaneous routes.

Administration of the agents identified in accordance with the present invention may be continuous or intermittent, depending, for example, upon the recipient's physiological condition, whether the purpose of the administration is therapeutic or prophylactic, and other factors known to skilled practitioners. The administration of the agents of the invention may be essentially continuous over a preselected period of time or may be in a series of spaced doses. Both local and systemic administration is contemplated. When the agents of the invention are employed for prophylactic purposes, agents of the invention are amenable to chronic use, preferably by systemic administration.

One or more suitable unit dosage forms comprising the agents of the invention, which, as discussed below, may optionally be formulated for sustained release, can be administered by a variety of routes including oral, or parenteral, including by rectal, transdermal, subcutaneous, intravenous, intramuscular, intraperitoneal, intrathoracic, intrapulmonary and intranasal routes. For example, for administration to the liver, intravenous administration is preferred. For administration to the lung, airway administration is preferred. The formulations may, where appropriate, be conveniently presented in discrete unit dosage forms and may be prepared by any of the methods well known to pharmacy. Such methods may include the step of bringing into association the agent with liquid carriers, solid matrices, semi-solid carriers, finely divided solid carriers or combinations thereof, and then, if necessary, introducing or shaping the product into the desired delivery system.

The active agent may be administered intravenously or intraperitoneally by infusion or injection. Solutions of the active agent or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion can include sterile aqueous solutions or dispersions or sterile powders comprising the active ingredient which are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the active agent in the required amount in the appropriate solvent with various of the other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

When the agents of the invention are prepared for oral administration, they are preferably combined with a pharmaceutically acceptable carrier, diluent or excipient to form a pharmaceutical formulation, or unit dosage form. The total active ingredients in such formulations comprise from 0.1 to 99.9% by weight of the formulation. By “pharmaceutically acceptable” it is meant the carrier, diluent, excipient, and/or salt must be compatible with the other ingredients of the formulation, and not deleterious to the recipient thereof. The active ingredient for oral administration may be present as a powder or as granules; as a solution, a suspension or an emulsion; or in achievable base such as a synthetic resin for ingestion of the active ingredients from a chewing gum. The active ingredient may also be presented as a bolus, electuary or paste.

The agents may be systemically administered, e.g., orally, in combination with a pharmaceutically acceptable vehicle such as an inert diluent or an assimilable edible carrier. They may be enclosed in hard or soft shell gelatin capsules, may be compressed into tablets, or may be incorporated directly with the food of the patient's diet. For oral administration, the active agent may be combined with one or more excipients and used in the form of ingestible tablets, buccal tablets, troches, capsules, elixirs, suspensions, syrups, wafers, and the like. Such compositions and preparations should contain at least 0.1% of active agent. The percentage of the compositions and preparations may, of course, be varied and may conveniently be between about 2 to about 60% of the weight of a given unit dosage form. The amount of active agent in such useful compositions is such that an effective dosage level will be obtained.

Pharmaceutical formulations containing the agents of the invention can be prepared by procedures known in the art using well known and readily available ingredients. For example, the agent can be formulated with common excipients, diluents, or carriers, and formed into tablets, capsules, suspensions, powders, and the like. Examples of excipients, diluents, and carriers that are suitable for such formulations include the following fillers and extenders such as starch, sugars, mannitol, and silicic derivatives; binding agents such as carboxymethyl cellulose, HPMC and other cellulose derivatives, alginates, gelatin, and polyvinyl-pyrrolidone; moisturizing agents such as glycerol; disintegrating agents such as calcium carbonate and sodium bicarbonate; agents for retarding dissolution such as paraffin; resorption accelerators such as quaternary ammonium compounds; surface active agents such as cetyl alcohol, glycerol monostearate; adsorptive carriers such as kaolin and bentonite; and lubricants such as talc, calcium and magnesium stearate, and solid polyethyl glycols.

For example, tablets or caplets containing the agents of the invention can include buffering agents such as calcium carbonate, magnesium oxide and magnesium carbonate. Caplets and tablets can also include inactive ingredients such as cellulose, pregelatinized starch, silicon dioxide, hydroxy propyl methyl cellulose, magnesium stearate, microcrystalline cellulose, starch, talc, titanium dioxide, benzoic acid, citric acid, corn starch, mineral oil, polypropylene glycol, sodium phosphate, and zinc stearate, and the like. Hard or soft gelatin capsules containing an agent of the invention can contain inactive ingredients such as gelatin, microcrystalline cellulose, sodium lauryl sulfate, starch, talc, and titanium dioxide, and the like, as well as liquid vehicles such as polyethylene glycols (PEGs) and vegetable oil. Moreover, enteric coated caplets or tablets of an agent of the invention are designed to resist disintegration in the stomach and dissolve in the more neutral to alkaline environment of the duodenum.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound may be incorporated into sustained-release preparations and devices.

The agents of the invention can also be formulated as elixirs or solutions for convenient oral administration or as solutions appropriate for parenteral administration, for instance by intramuscular, subcutaneous or intravenous routes.

The pharmaceutical formulations of the agents of the invention can also take the form of an aqueous or anhydrous solution or dispersion, or alternatively the form of an emulsion or suspension.

Thus, the therapeutic agent may be formulated for parenteral administration (e.g., by injection, for example, bolus injection or continuous infusion) and may be presented in unit dose form in ampules, pre-filled syringes, small volume infusion containers or in multi-dose containers with an added preservative. The active ingredients may take such forms as suspensions, solutions, or emulsions in oily or aqueous vehicles, and may contain formulatory agents such as suspending, stabilizing and/or dispersing agents. Alternatively, the active ingredients may be in powder form, obtained by aseptic isolation of sterile solid or by lyophilization from solution, for constitution with a suitable vehicle, e.g., sterile, pyrogen-free water, before use.

These formulations can contain pharmaceutically acceptable vehicles and adjuvants which are well known in the prior art. It is possible, for example, to prepare solutions using one or more organic solvent(s) that is/are acceptable from the physiological standpoint, chosen, in addition to water, from solvents such as acetone, ethanol, isopropyl alcohol, glycol ethers such as the products sold under the name “Dowanol”, polyglycols and polyethylene glycols, C₁-C₄ alkyl esters of short-chain acids, preferably ethyl or isopropyl lactate, fatty acid triglycerides such as the products marketed under the name “Miglyol”, isopropyl myristate, animal, mineral and vegetable oils and polysiloxanes.

The compositions according to the invention can also contain thickening agents such as cellulose and/or cellulose derivatives. They can also contain gums such as xanthan, guar or carbo gum or gum arabic, or alternatively polyethylene glycols, bentones and montmorillonites, and the like.

It is possible to add, if necessary, an adjuvant chosen from antioxidants, surfactants, other preservatives, film-forming, keratolytic or comedolytic agents, perfumes and colorings. Also, other active ingredients may be added, whether for the conditions described or some other condition.

For example, among antioxidants, t-butylhydroquinone, butylated hydroxyanisole, butylated hydroxytoluene and α-tocopherol and its derivatives may be mentioned. The galenical forms chiefly conditioned for topical application take the form of creams, milks, gels, dispersion or microemulsions, lotions thickened to a greater or lesser extent, impregnated pads, ointments or sticks, or alternatively the form of aerosol formulations in spray or foam form or alternatively in the form of a cake of soap.

Additionally, the agents are well suited to formulation as sustained release dosage forms and the like. The formulations can be so constituted that they release the active ingredient only or preferably in a particular part of the intestinal or respiratory tract, possibly over a period of time. The coatings, envelopes, and protective matrices may be made, for example, from polymeric substances, such as polylactide-glycolates, liposomes, microemulsions, microparticles, nanoparticles, or waxes. These coatings, envelopes, and protective matrices are useful to coat indwelling devices, e.g., stents, catheters, peritoneal dialysis tubing, and the like. The agents of the invention can be delivered via patches for transdermal administration. See U.S. Pat. No. 5,560,922 for examples of patches suitable for transdermal delivery of an agent. Patches for transdermal delivery can comprise a backing layer and a polymer matrix which has dispersed or dissolved therein an agent, along with one or more skin permeation enhancers. The backing layer can be made of any suitable material which is impermeable to the agent. The backing layer serves as a protective cover for the matrix layer and provides also a support function. The backing can be formed so that it is essentially the same size layer as the polymer matrix or it can be of larger dimension so that it can extend beyond the side of the polymer matrix or overlay the side or sides of the polymer matrix and then can extend outwardly in a manner that the surface of the extension of the backing layer can be the base for an adhesive means. Alternatively, the polymer matrix can contain, or be formulated of, an adhesive polymer, such as polyacrylate or acrylate/vinyl acetate copolymer. For long-term applications it might be desirable to use microporous and/or breathable backing laminates, so hydration or maceration of the skin can be minimized.

Examples of materials suitable for making the backing layer are films of high and low density polyethylene, polypropylene, polyurethane, polyvinylchloride, polyesters such as poly(ethylene phthalate), metal foils, metal foil laminates of such suitable polymer films, and the like. Preferably, the materials used for the backing layer are laminates of such polymer films with a metal foil such as aluminum foil. In such laminates, a polymer film of the laminate will usually be in contact with the adhesive polymer matrix.

The backing layer can be any appropriate thickness which will provide the desired protective and support functions. A suitable thickness will be from about 10 to about 200 microns.

Generally, those polymers used to form the biologically acceptable adhesive polymer layer are those capable of forming shaped bodies, thin walls or coatings through which agents can pass at a controlled rate. Suitable polymers are biologically and pharmaceutically compatible, nonallergenic and insoluble in and compatible with body fluids or tissues with which the device is contacted. The use of soluble polymers is to be avoided since dissolution or erosion of the matrix by skin moisture would affect the release rate of the agents as well as the capability of the dosage unit to remain in place for convenience of removal.

Exemplary materials for fabricating the adhesive polymer layer include polyethylene, polypropylene, polyurethane, ethylene/propylene copolymers, ethylene/ethylacrylate copolymers, ethylene/vinyl acetate copolymers, silicone elastomers, especially the medical-grade polydimethylsiloxanes, neoprene rubber, polyisobutylene, polyacrylates, chlorinated polyethylene, polyvinyl chloride, vinyl chloride-vinyl acetate copolymer, crosslinked polymethacrylate polymers (hydrogel), polyvinylidene chloride, poly(ethylene terephthalate), butyl rubber, epichlorohydrin rubbers, ethylene vinyl alcohol copolymers, ethylene-vinyloxyethanol copolymers; silicone copolymers, for example, polysiloxane-polycarbonate copolymers, polysiloxane-polyethylene oxide copolymers, polysiloxane-polymethacrylate copolymers, polysiloxane-alkylene copolymers (e.g., polysiloxane-ethylene copolymers), polysiloxane-alkylenesilane copolymers (e.g., polysiloxane-ethylenesilane copolymers), and the like; cellulose polymers, for example methyl or ethyl cellulose, hydroxy propyl methyl cellulose, and cellulose esters; polycarbonates; polytetrafluoroethylene; and the like.

Preferably, a biologically acceptable adhesive polymer matrix should be selected from polymers with glass transition temperatures below room temperature. The polymer may, but need not necessarily, have a degree of crystallinity at room temperature. Cross-linking monomeric units or sites can be incorporated into such polymers. For example, cross-linking monomers can be incorporated into polyacrylate polymers, which provide sites for cross-linking the matrix after dispersing the agent into the polymer. Known cross-linking monomers for polyacrylate polymers include polymethacrylic esters of polyols such as butylene diacrylate and dimethacrylate, trimethylol propane trimethacrylate and the like. Other monomers which provide such sites include allyl acrylate, allyl methacrylate, diallyl maleate and the like.

Preferably, a plasticizer and/or humectant is dispersed within the adhesive polymer matrix. Water-soluble polyols are generally suitable for this purpose. Incorporation of a humectant in the formulation allows the dosage unit to absorb moisture on the surface of skin which in turn helps to reduce skin irritation and to prevent the adhesive polymer layer of the delivery system from failing.

Agents released from a transdermal delivery system must be capable of penetrating each layer of skin. In order to increase the rate of permeation of an agent, a transdermal drug delivery system must be able in particular to increase the permeability of the outermost layer of skin, the stratum corneum, which provides the most resistance to the penetration of molecules. The fabrication of patches for transdermal delivery of agents is well known to the art.

For administration to the upper (nasal) or lower respiratory tract by inhalation, the agents of the invention are conveniently delivered from an insufflator, nebulizer or a pressurized pack or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount.

Alternatively, for administration by inhalation or insufflation, the composition may take the form of a dry powder, for example, a powder mix of the agent and a suitable powder base such as lactose or starch. The powder composition may be presented in unit dosage form in, for example, capsules or cartridges, or, e.g., gelatine or blister packs from which the powder may be administered with the aid of an inhalator, insufflator or a metered-dose inhaler.

For intra-nasal administration, the agent may be administered via nose drops, a liquid spray, such as via a plastic bottle atomizer or metered-dose inhaler. Typical of atomizers are the Mistometer (Wintrop) and the Medihaler (Riker).

The local delivery of the agents of the invention can also be by a variety of techniques which administer the agent at or near the site of disease. Examples of site-specific or targeted local delivery techniques are not intended to be limiting but to be illustrative of the techniques available. Examples include local delivery catheters, such as an infusion or indwelling catheter, e.g., a needle infusion catheter, shunts and stents or other implantable devices, site specific carriers, direct injection, or direct applications.

For topical administration, the agents may be formulated as is known in the art for direct application to a target area. The agents may be applied in pure form, i.e., when they are liquids. However, it will generally be desirable to administer them to the skin as compositions or formulations, in combination with a dermatologically acceptable carrier, which may be a solid or a liquid. Conventional forms for this purpose include wound dressings, coated bandages or other polymer coverings, ointments, creams, lotions, pastes, jellies, sprays, and aerosols. Ointments and creams may, for example, be formulated with an aqueous or oily base with the addition of suitable thickening and/or gelling agents. Lotions may be formulated with an aqueous or oily base and will in general also contain one or more emulsifying agents, stabilizing agents, dispersing agents, suspending agents, thickening agents, or coloring agents. The active ingredients can also be delivered via iontophoresis, e.g., as disclosed in U.S. Pat. No. 4,140,122; 4,383,529; or 4,051,842. The percent by weight of an agent of the invention present in a topical formulation will depend on various factors, but generally will be from 0.01% to 95% of the total weight of the formulation, and typically 0.1-25% by weight.

Useful solid carriers include finely divided solids such as talc, clay, microcrystalline cellulose, silica, alumina and the like. Useful liquid carriers include water, alcohols or glycols or water-alcohol/glycol blends, in which the present compounds can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Drops, such as eye drops or nose drops, may be formulated with an aqueous or non-aqueous base also comprising one or more dispersing agents, solubilizing agents or suspending agents. Liquid sprays are conveniently delivered from pressurized packs. Drops can be delivered via a simple eye dropper-capped bottle, or via a plastic bottle adapted to deliver liquid contents dropwise, via a specially shaped closure.

The agent may further be formulated for topical administration in the mouth or throat. For example, the active ingredients may be formulated as a lozenge further comprising a flavored base, usually sucrose and acacia or tragacanth; pastilles comprising the composition in an inert base such as gelatin and glycerin or sucrose and acacia; and mouthwashes comprising the composition of the present invention in a suitable liquid carrier.

The formulations and compositions described herein may also contain other ingredients such as antimicrobial agents, or preservatives. Furthermore, the active ingredients may also be used in combination with other agents, for example, bronchodilators.

The agents of this invention may be administered to a mammal alone or in combination with pharmaceutically acceptable carriers. As noted above, the relative proportions of active ingredient and carrier are determined by the solubility and chemical nature of the compound, chosen route of administration and standard pharmaceutical practice.

The dosage of the present agents will vary with the form of administration, the particular compound chosen and the physiological characteristics of the particular patient under treatment. Generally, small dosages will be used initially and, if necessary, will be increased by small increments until the optimum effect under the circumstances is reached.

Useful dosages of the agents can be determined by comparing their in vitro activity and in vivo activity in animal models. Methods for the extrapolation of effective dosages in mice, and other animals, to humans are known to the art; for example, see U.S. Pat. No. 4,938,949.

Generally, the concentration of the agent in a liquid composition, such as a lotion, will be from about 0.1-25 wt-%, preferably from about 0.5-10 wt-%. The concentration in a semi-solid or solid composition such as a gel or a powder will be about 0.1-5 wt-%, preferably about 0.5-2.5 wt-%.

The amount of the agent, or an active salt or derivative thereof, required for use alone or with other agents will vary not only with the particular salt selected but also with the route of administration, the nature of the condition being treated and the age and condition of the patient and will be ultimately at the discretion of the attendant physician or clinician.

The agent may be conveniently administered in unit dosage form; for example, containing 5 to 1000 mg, conveniently 10 to 750 mg, most conveniently, 50 to 500 mg of active ingredient per unit dosage form.

In general, however, a suitable dose may be in the range of from about 0.5 to about 100 mg/kg, e.g., from about 10 to about 75 mg/kg of body weight per day, such as 3 to about 50 mg per kilogram body weight of the recipient per day, preferably in the range of 6 to 90 mg/kg/day, most preferably in the range of 15 to 60 mg/kg/day. An apocynin containing composition may contain at least 50 μg, preferably at least 100 μg, up to 1000 mg of apocynin on the basis of daily intake. An example daily intake is between 1 and 100 mg apocynin; preferably a dosage of at least 15 mg/day. For instance, apocynin may be orally administered as a root powder in a dose of 375 mg three times in a day, by intramuscular injection of an alcoholic extract of the root of the plant daily (40 mg/kg) or by aerosol delivery administered in 8 doses for a total of 2 mg. An exemplary formulation and dosage include 300 to 500 mg root powder b.i.d./t.i.d. Moreover, analogs of apocynin may be used instead of or in addition to apocynin. Such analogs are in particular those in which the 4-hydroxyl group is etherified, especially with a hydroxylated alkyl group, such as 2-hydroxyethyl, 2,3-dihydroxypropyl or a sugar moiety. The latter analog in which the sugar moiety is β-D-glucose, is commonly known as androsin. This is the usual form in which apocynin is present in fresh plants.

The active ingredient may be administered to achieve peak plasma concentrations of the active compound of from about 0.5 to about 75 μM, preferably, about 1 to 50 μM, most preferably, about 2 to about 30 μM. This may be achieved, for example, by the intravenous injection of a 0.05 to 5% solution of the active ingredient, optionally in saline, or orally administered as a bolus containing about 1-100 mg of the active ingredient. Desirable blood levels may be maintained by continuous infusion to provide about 0.01-5.0 mg/kg/hr or by intermittent infusions containing about 0.4-15 mg/kg of the active ingredient(s).

The desired dose may conveniently be presented in a single dose or as divided doses administered at appropriate intervals, for example, as two, three, four or more sub-doses per day. The sub-dose itself may be further divided, e.g., into a number of discrete loosely spaced administrations; such as multiple inhalations from an insufflator or by application of a plurality of drops into the eye.

### Reagents to Isolate Endosomal Preparations

The present invention generally provides a method of isolating endosomes. In one embodiment, the method may employ recombinant cells transfected with exogenous nucleic acid having an expression cassette encoding a Rac fusion protein. The method may employ a cell which expresses the Rac fusion protein from an expression cassette which is either transiently or stably introduced to the cell. The expression cassette includes a promoter driving expression of the fusion protein. The promoter may be a constitutive promoter or a regulatable promoter, e.g., inducible.

In one embodiment, the Rae peptide or polypeptide is one which is fused to other sequences, e.g., a glutathione S-transferase (GST) sequence, a His tag, calmodulin binding peptide, tobacco etch virus protease, protein A IgG binding domain, and the like, or a combination of sequences, useful to isolate, purify or detect the linked Rac polypeptide. In one embodiment, His-Rac1 is immobilized on a support, e.g., a multi-well plate.

To prepare expression cassettes encoding a Rac fusion for transformation, the recombinant DNA sequence or segment may be circular or linear, double-stranded or single-stranded. A DNA sequence which encodes an RNA sequence that is substantially complementary to a mRNA sequence encoding a gene product of interest is typically a “sense” DNA sequence cloned into a cassette in the opposite orientation (i.e., 3′ to 5′ rather than 5′ to 3′). Generally, the DNA sequence or segment is in the form of chimeric DNA, such as plasmid DNA, that can also contain coding regions flanked by control sequences which promote the expression of the DNA in a cell. As used herein, “chimeric” means that a vector comprises DNA from at least two different species, or comprises DNA from the same species, which is linked or associated in a manner which does not occur in the “native” or wild-type of the species.

Aside from DNA sequences that serve as transcription units, or portions thereof, a portion of the DNA may be untranscribed, serving a regulatory or a structural function. For example, the DNA may itself comprise a promoter that is active in eukaryotic cells, e.g., mammalian cells, or in certain cell types, or may utilize a promoter already present in the genome that is the transformation target of the lymphotrophic virus. Such promoters include the CMV promoter, as well as the SV40 late promoter and retroviral LTRs (long terminal repeat elements), although many other promoter elements well known to the art may be employed, e.g., the MMTV, RSV, MLV or HIV LTR in the practice of the invention.

Other elements functional in the host cells, such as introns, enhancers, polyadenylation sequences and the like, may also be a part of the recombinant DNA. Such elements may or may not be necessary for the function of the DNA, but may provide improved expression of the DNA by affecting transcription, stability of the mRNA, or the like. Such elements may be included in the DNA as desired to obtain the optimal performance of the transforming DNA in the cell.

The recombinant DNA to be introduced into the cells may contain either a selectable marker gene or a reporter gene or both to facilitate identification and selection of transformed cells from the population of cells sought to be transformed. Alternatively, the selectable marker may be carried on a separate piece of DNA and used in a co-transformation procedure. Both selectable markers and reporter genes may be flanked with appropriate regulatory sequences to enable expression in the host cells. Useful selectable markers are well known in the art and include, for example, antibiotic and herbicide-resistance genes, such as neo, hpt, dhfr, bar, aroA, puro, hyg, dapA and the like. See also, the genes listed on Table 1 of Lundquist et al. (U.S. Pat. No. 5,848,956).

Reporter genes are used for identifying potentially transformed cells and for evaluating the functionality of regulatory sequences. Reporter genes which encode for easily assayable proteins are well known in the art. In general, a reporter gene is a gene which is not present in or expressed by the recipient organism or tissue and which encodes a protein whose expression is manifested by some easily detectable property, e.g., enzymatic activity. Exemplary reporter genes include the chloramphenicol acetyl transferase gene (cat) from Tn9 of E. coli, the beta-glucuronidase gene (gus) of the uidA locus of E. coli, the green, red, or blue fluorescent protein gene, and the luciferase gene. Expression of the reporter gene is assayed at a suitable time after the DNA has been introduced into the recipient cells.

The general methods for constructing recombinant DNA which can transform target cells are well known to those skilled in the art, and the same compositions and methods of construction may be utilized to produce the DNA useful herein.

The recombinant DNA can be readily introduced into the host cells, e.g., mammalian, bacterial, yeast or insect cells, or prokaryotic cells, by transfection with an expression vector comprising the recombinant DNA by any procedure useful for the introduction into a particular cell, e.g., physical or biological methods, to yield a transformed (transgenic) cell having the recombinant DNA so that the DNA sequence of interest is expressed by the host cell. In one embodiment, the recombinant DNA is stably integrated into the genome of the cell.

Physical methods to introduce a recombinant DNA into a host cell include calcium-mediated methods, lipofection, particle bombardment, microinjection, electroporation, and the like. Biological methods to introduce the DNA of interest into a host cell include the use of DNA and RNA viral vectors. Viral vectors, e.g., retroviral or lentiviral vectors, have become a widely used method for inserting genes into eukaryotic cells, such as mammalian, e.g., human cells. Other viral vectors can be derived from poxviruses, e.g., vaccinia viruses, herpes viruses, adenoviruses, adeno-associated viruses, baculoviruses, and the like.

To confirm the presence of a recombinant DNA sequence in the host cell, a variety of assays may be performed. Such assays include, for example, molecular biological assays well known to those of skill in the art, such as Southern and Northern blotting, RT-PCR and PCR; biochemical assays, such as detecting the presence or absence of a particular gene product, e.g., by immunological means (ELISAs and Western blots) or by other molecular assays.

To detect and quantitate RNA produced from introduced recombinant DNA segments, RT-PCR may be employed. In this application of PCR, it is first necessary to reverse transcribe RNA into DNA, using enzymes such as reverse transcriptase, and then through the use of conventional PCR techniques amplify the DNA. In most instances PCR techniques, while useful, will not demonstrate integrity of the RNA product. Further information about the nature of the RNA product may be obtained by Northern blotting. This technique demonstrates the presence of an RNA species and gives information about the integrity of that RNA. The presence or absence of an RNA species can also be determined using dot or slot blot Northern hybridizations. These techniques are modifications of Northern blotting and only demonstrate the presence or absence of an RNA species.

While Southern blotting and PCR may be used to detect the recombinant DNA segment in question, they do not provide information as to whether the recombinant DNA segment is being expressed. Expression may be evaluated by specifically identifying the peptide products of the introduced DNA sequences or evaluating the phenotypic changes brought about by the expression of the introduced DNA segment in the host cell.

The invention will be further described by the following nonlimiting Examples.

### EXAMPLE I

### Methods

### Virus, Cell Culture and Viral Infection

Recombinant type-2 adeno-associated viruses encoding luciferase (AV2Luc) or factor VIII (AV2FVIII) transgenes were used for infection of different cell types. HeLa and IB3 cells were cultured in DMEM supplemented with 10% fetal bovine serum (FBS) and antibiotics. Nox1 and Nox2 knockout and wild-type (wt) littermate control mouse dermal fibroblasts were generated from newborn mice as described in Bosu et al. (2001) and below. For all infections, viruses were applied to cells in serum-free DMEM, and an equal volume of 20% FBS/DMEM was added at 2 hours post-infection.

In studies evaluating the efficiency of viral transduction, AV2Luc virus was used at a multiplicity of infection (MOI) equal to 1,000 particles/cell, and luciferase activity was measured at 24 hours post-infection. In studies evaluating viral entry, cells were incubated with 1,000 particles/cell of AV2FVIII at 4° C. for 30 minutes prior to removing virus, washing cells, and shifting cells to 37° C. Cells were then incubated for the indicated times and postnuclear supernatants (PNS) were prepared for analysis of intracellular AAV2. Taqman PCR was used to quantify the abundance of viral genomes in different subcellular fractions following infection using primer sets and methods described in Ding et al. (2006). When proteasome inhibitors were added to induce AAV2 transduction, LLnL (Boston Biochem, Cambridge, Mass.) and/or doxorubicin (Calbiochem, San Diego, Calif.), were present in the medium at concentrations of 40 μM and 5 μM, respectively. Modulation of endosomal ROS was achieved by adding 1 mg/mL of purified bovine Cu/Zn superoxide dismutase (Cu/ZnSOD) (Oxis Research, Portland, Oreg.) and/or catalase (Sigma-Aldrich, St. Louis, Mo.) to the medium which was then applied to cells 20 minutes prior to viral infections unless indicated otherwise. SOD and/or catalase remained in the medium during infection unless otherwise indicated.

### Vesicular Fractionation and Assays for NADPH-Dependent Superoxide Production

Vesicular fractionation was performed using a previously described protocol (Li et al., 2006b) with minor modifications. Briefly, cells were harvested by scraping and washing twice in 4° C. phosphate-buffered saline (PBS). Cells were then pelleted and resuspended on ice in 1 mL of pre-cooled (4° C.) homogenization buffer (0.25 M sucrose, 10 mM triethanolamine, 1 mM EDTA, 1 mM PMSF, and 100 μg/mL aprotinin) and homogenized using a nitrogen decompression vessel (Parr Instrument, Moline, Ill.). Methods for generating PNS, iodixanol isolation of vesicular fractions, sample collection, quality control, and Nox activity assays were performed as described in Li et al. (2006b).

In brief, vesicular fractions were confirmed by Western blot to contain known endosomal Rab proteins and be devoid of markers for plasma membrane and mitochondria. Nox activities were analyzed by measuring the rate of .O₂⁻ generation using a chemiluminescent, lucigenin-based system. Prior to the initiation of the assay, vesicular fractions were combined with 5 μM lucigenin (Sigma-Aldrich, St. Louis, Mo.) in PBS and incubated in darkness at room temperature for 10 minutes. The reaction was initiated by the addition of 100 μM of NADPH (Sigma-Aldrich, St. Louis, Mo.) and changes in luminescence were measured over the course of 3 min (5 readings/sec). The slope of the luminescence curve (relative light units [RLU] per minute) (r>0.95) was used to calculate the rate of .O₂⁻ formation as an index of NADPH oxidase activity.

Alexa546-Labeling of rAAV2 and Fluorescent Microscopy

Approximately 2×10¹² purified AV2FVIII virions were diluted in 500 μL conjugation buffer (0.1 M sodium carbonate, pH 9.3), incubated at room temperature for 1 hour with 50 nM of Alexa546 reactive dye (Invitrogen, Carlsbad, Calif.) and mixed continuously. To stop the reaction, 1 mL of 10 mM Tris (pH 8.0) was added to the solution. The labeled virus was then purified using ion-exchange high-performance liquid chromatography as described in Kaludov et al. (2002). The final concentration of purified labeled virus was 1×10¹² particles/mL, as determined by slot blot hybridization using a viral DNA probe. EGFP-Rac1 plasmid was a generous gift from Dr. Klaus Hahn and was transfected into HeLa cells following a standard electroporation protocol. At 36 hours post-transfection, transfected HeLa cells were pre-cooled to 4° C. for 10 minutes, followed by incubating cells for 1 hour at 4° C. with Alexa546-labeled AV2FVIII at an MOI of 104 particles/cell. Unbound virus was then removed by washing with fresh media, and viral entry was initiated by shifting cells to 37° C. for indicated periods of time. Cells were then washed with PBS four times prior to fixation in 4% paraformaldehyde. Fixed cells were mounted with VectraShield mounting media, and were examined with a Yokogawa CSU10 confocal microscope.

### In Vivo Study Evaluating AAV2-Mediated Gene Transfer to Nox2 Knockout (KO) Mice

Nox2 KO and the littermate wild type mice on the C57BL6 background were lightly anesthetized in a methoxyflurane chamber. 1×10¹¹ particles of AV2Luc were administered with 20 μM Doxorubin/PBS in a 40 μL volume by nasal aspiration. Mice were euthanized at 2 weeks post-infection and the lungs were collected for luciferase expression assays.

### In Vitro Phospholipase A₂ (PLA₂) Activity Assays Using Purified AAV2 Virus

10¹⁰ purified AV2FVIII virions were treated with various conditions to activate PLA₂ activity in the capsid of purified virions. This included pre-incubation of virus with various concentration of H₂O₂ for 15 minutes at 37° C. or partial heat denaturation at 70° C. for 2 minutes. Catalase was added to virus treated with H₂O₂ at the end of the treatment period to scavenge H₂O₂ prior to the PLA₂ activity assay. PLA₂ activity of virions was determined by the release of radioactive fatty acid from L-3-phosphatidylcholine (PC), 1,2-di[1-¹⁴C]oleoyl using the protocol described by Zadori et al. (2001).

### Trypsin Sensitivity Assays of AAV2 Virions Using MALDI-TOF Mass Spectrometry

Subtle changes in capsid structure of the virion following heat denaturation or exposure to H₂O₂ was assayed using MALDI-TOF mass spectrometry following trypsin digestion. Briefly, 10¹⁰ AAV2 particles were treated with H₂O₂ (which was then removed by dialysis for 1 hour against 20 mM Tris, 20 mM NaCl, pH 8.0) or heated to 70° C. as described above and then incubated with 500 ng of porcine trypsin in 25 mM NH₄HCO₃ (pH 8.0) at 37° C. for 16 hours. The digested products were then sequentially incubated with 10 mM DTT and 55 mM of iodoacetamide to reduce and modify cysteine residues. 1/20 of the original sample was assayed on a Bruker Biflex III MALDI-TOF MS. For the analysis of cysteine modification, the theoretical m/z values of peptides containing individual cysteines were predicted without modification, with iodoacetamide modification, and with different oxidative (sulfenic, sulfinic, or sulfonic) modifications. Spectra were evaluated using ExPASy (http://us.expasy.org/tools/peptide-mass.html) and compared to the theoretical spectra for various VP proteins.

### Assay for Virion Endosomal Escape

A protocol using a 30% iodixanol cushion was developed to separate AAV2 virions in the cytoplasm from those inside endosomes. Briefly, HeLa cells were incubated with 1,000 particles/cell of AAV2 at 4° C. for 30 minutes before removing virus, washing cells, and shifting cells to 37° C. Cells were then incubated for the indicated times and PNS were prepared. A total volume of 500 μl PNS was then loaded on the top of 250 μl 30% iodixanol, followed by centrifugation at 100,000×g for 1 hour. Viral genome within the supernatant and pellet were quantified by real-time PCR.

### Generation of C298S Capsid Mutant AAV2

The capsid domain that contains C289 was cloned from pAV2RepCap into pBluescript II SK (Stratagene, La Jolla, Calif.) using Kpn I. The resultant plasmid, pBluescriptAV2Cap, was used to perform the C289S mutagenesis using the QuickChange Site-Directed Mutagenesis kit (Stratagene). The capsid domain with the C289S mutation was then cloned back into pAV2RepCap using Kpn I, resulting in pAV2RepCapC289S. The mutation was then confirmed by sequencing. Recombinant AAV2 encoding luciferase was generated following a triple plasmid transfection protocol described in Yan et al. (2002) with pAV2RepCap or pAV2RepCapC289S providing either wild type or C298S capsid, respectively. Recombinant AAV2 was purified from both vectors using a standard protocols previously described in Yan et al. (2002). Viral titers were determined by real-time PCR and slot blot hybridization. The titers of purified virus were 6.5×10¹¹ particle/ml for AAV2 with wt capsid, and 5×10¹¹ particles/ml for the AAV2-C289S capsid.

### NADPH Oxidase Deficient Mice and Dermal Fibroblasts.

Nox1 and Nox2 knockout lines used in these studies have been described in Pollock et al. (1995) and Gavazzi et al. (2000). In all comparative studies KO and WT littermate control were used. Mouse dermal fibroblasts were generated from newborn mice as described in Basu et al. (2001). Briefly, to establish the culture of primary dermal fibroblasts, the skin was removed from newborn mice and incubated in 0.25% trypsin-EDTA overnight at 4° C. The dermis layer of the skin was then separated from the epidermis and incubated in 0.2% collagenase in DMEM for 1 hour at 37° C. followed by vigorous shaking to release the fibroblasts. The released fibroblasts were then pelleted, resuspended, and maintained in culture in DMEM supplemented with 10% FBS, 2 mM L-Glutamine, and antibiotics.

### Results

Adeno-associated virus (AAV) is a small single stranded DNA parvovirus most commonly known for it use as a gene therapy vector (Carter, 2005). Its simple 4.7 kb genome encodes two viral genes, Rep and Cap, that are required for replication and encapsidation of its genome. Recombinant AAV (rAAV) has been extensively studied as a gene therapy vector and clinical trials using this vector are growing rapidly. As such, the processes of AAV infection are being increasing studied in an attempt to dissect the biology of the over 10 serotypes thus far identified. The most well studied serotype to date is AAV type 2. As with many of types of viruses, redox stress by UV irradiation or H₂O₂ is known to increase AAV2 transduction and anti-oxidants such as N-acetyl-L-cysteine (NAC) inhibit transduction (Sanlioglu et al., 2004; Sanlioglu et al., 1999). However, the redox-regulated events responsible for this observation remain unknown. As described below, the mechanism responsible for ROS mediated transduction of rAAV-2 was dissected.

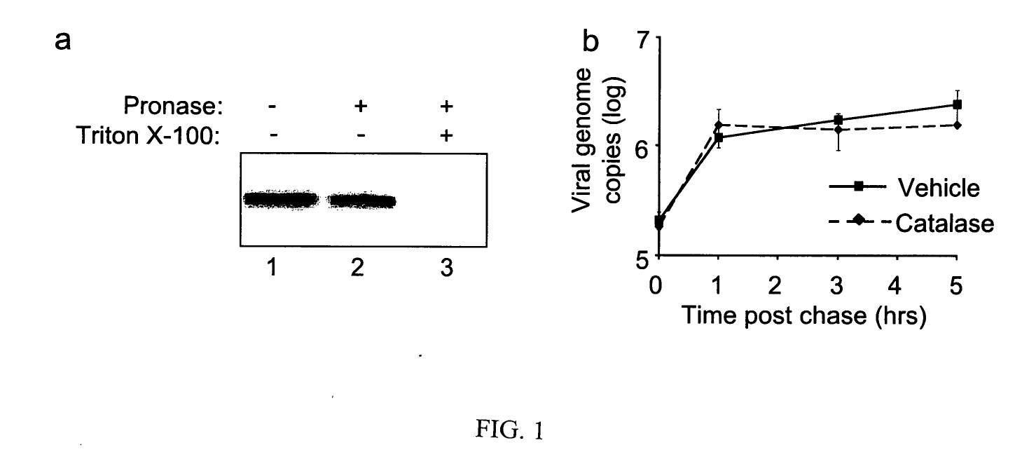

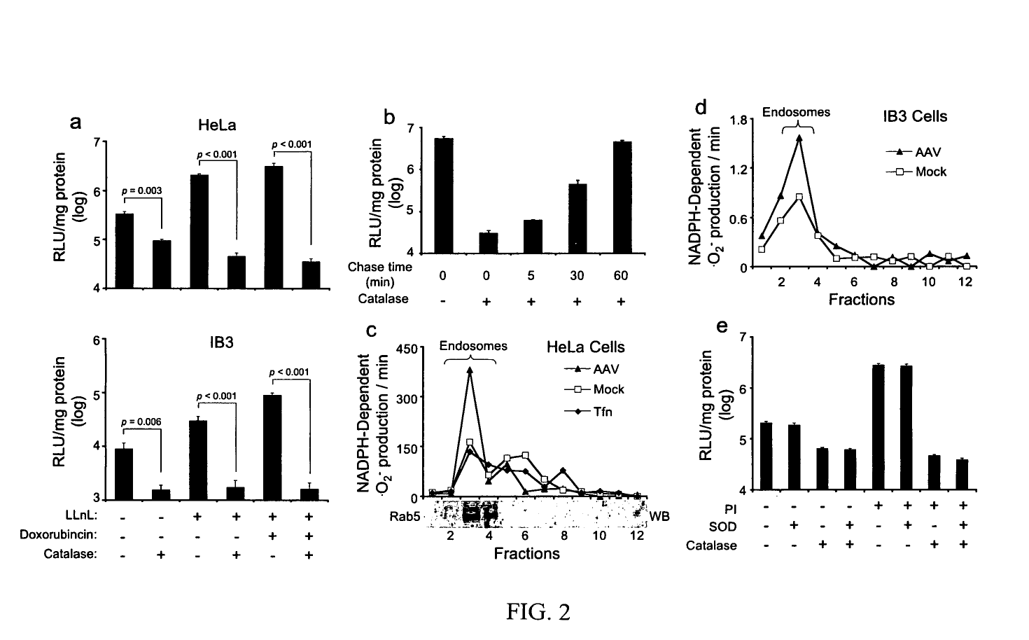

Endosomal trafficking and intracellular processing have been regarded as rate-limiting steps for AAV2 transduction (Duan et al., 2000; Hansen et al., 2000; Hauck et al., 2004). In this context, proteasome inhibition dramatically enhances AAV transduction in vitro and in vivo by increasing nuclear uptake of virus through an as yet a poorly defined mechanism. Given that endosomal processing of AAV2 is inefficient and H₂O₂ is known to enhance AAV2 transduction, endosomal ROS might be important for processing of the virions following infection. To this end, it was tested whether clearance of endosomal H₂O₂ by loading endosomes with purified catalase would inhibit transduction of AAV2. The addition of 1 mg/mL catalase to the media on HeLa cells led to the accumulation of pronase-insensitive catalase inside purified endosomes (FIG. 1A). Indeed, endosomal loading with catalase significantly inhibited AAV2 transduction of both IB3 (a transformed bronchial epithelial cell line) and HeLa cells, as reflected by the expression of a recombinant luciferase transgene (FIG. 2A). Strikingly, catalase loading also completely abolished the ability of proteasome inhibitors to enhance AAV2 transduction in both cell lines (Yan et al., 2004) (FIG. 2A). This inhibitory effect of endosomal catalase loading was not due to impaired viral uptake (FIG. 1B). Time course studies loading catalase at various times during and after infection suggested that catalase acts to inhibit AAV2 transduction at a relatively early stage of viral infection (FIG. 2B). By 30 minutes post-infection, the ability of catalase loading to inhibit viral transduction significantly declined and was completely absent by 60 minutes post-infection. This suggested that H₂O₂ acted to enhance transduction early in the infectious process.

A primary source of endosomal H₂O₂ important in cell signaling has recently been identified as being NADPH oxidases that produce .O₂⁻ (Li et al., 2006). In this context, .O₂⁻ dismutation leads to H₂O₂ formation and this reaction can occur spontaneously at a very rapid rate at low pH normally found in many endosomal compartments (Bielski et al., 1985). Therefore, AAV2 infection might stimulate endosomal Nox activity and hence superoxide production. Using a lucigenin-based chemiluminescent assay (Li et al., 2006), it was determined whether AAV2 infection promoted endosomal NADPH-dependent .O₂⁻ production in iodixanol-fractionated endosomes. Indeed, a 2-3 fold increase in NADPH-dependent .O₂⁻ production was seen in the vesicular fractions of both cells lines following AAV infection (FIGS. 2C-D). Additionally, virally induced NADPH-dependent .O₂⁻ production in the endosomal fraction was inhibited by DPI, a known inhibitor of NADPH oxidases (data not shown). These findings suggested that Nox activation in the endosomal compartment occurs following AAV2 infection. Interestingly, a correlation was observed in the permissiveness of these two cells lines for AAV2 infection and their ability to generate NADPH-dependent .O₂⁻ in the endosomal compartment; vesicular fractions of HeLa cells generated >100-fold higher levels .O₂⁻ than that of IB3 cells, which directly correlated with their relative levels of transduction with AAV2 vector (FIGS. 2A and C-D). These findings also supported the hypothesis that endosomal ROS positively influence AAV2 infection.

The data thus far suggested that H₂O₂ in the endosomal compartment was functionally important for AAV2 infection. However, given that .O₂⁻ and H₂O₂ can react to form hydroxyl radicals, it was unclear if H₂O₂ was the functionally important ROS mediating endosomal processing of AAV2. To address this question, endosomal loading experiments were performed with purified superoxide dismutase-1 (SOD1) and/or catalase. It was hypothesized that if .O₂⁻ was critical for AAV2 processing in the endosomal compartment, then the enhanced conversion of .O₂⁻→H₂O₂ by SOD1 would inhibit AAV2 transduction. However, loading of endosomes with purified SOD1 under conditions known to quench endosomal Nox-mediated .O₂⁻ production (Li et al., 2006b) failed to alter AAV2 transduction in the absence or presence of proteasome inhibitors (FIG. 2E). These findings suggested that .O₂⁻ is not necessary for productive AAV2 infection and that the rate of spontaneous dismutation of .O₂⁻→H₂O₂ is not limiting in the endosomal compartment important for AAV2 processing.

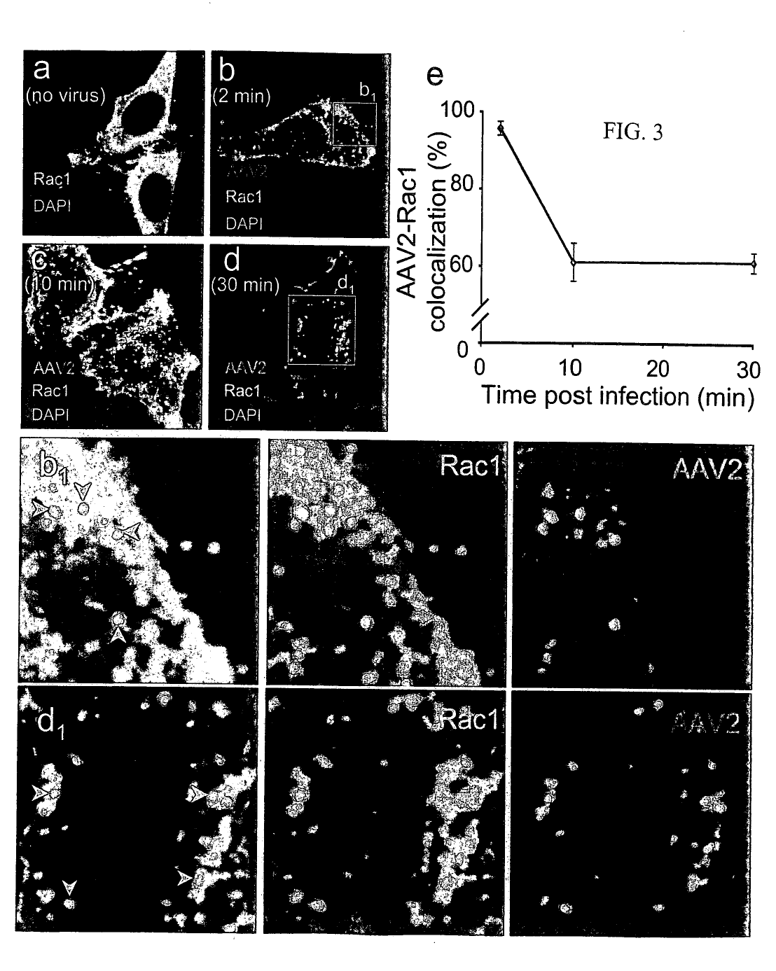

Rac1 GTPase has been reported to be a co-factor for the activation of both the Nox1 and Nox2 enzymatic complex (Lambeth, 2004; Park et al., 2004). In addition, AAV2 infection stimulates activation of Rac1-GTP and the dominant negative N17Rac1 mutant significantly inhibits AAV2 infection (Sanglioglu et al., 2000). These findings support the potential importance of Rac1-regulated Nox activation in AAV2 infection. To this end, it was evaluated whether AAV2 was directly endocytosed into Rac1 bound endosomes using GFP-Rac1 and Alexa546-labeled AAV2. In the absence of viral infection, Rac1 was primarily distributed evenly throughout the cytoplasm (FIG. 3A). Beginning at 2 minutes following viral infection, EGFP-Rac1 localization was seen to increase in endosomes co-localizing with Alexa546-labeled AAV2 and this co-localization progressively moved to vesicular structures in the perinuclear region by 30 minutes post-infection, a region known to accumulate AAV2 (FIGS. 3B-D). Interestingly, using quantitative morphometry a decline in the extent of vesicular AAV2-Rac1 colocalization was observed from 2 minutes (>95%) to 10 minutes (about 60%) post-infection. These morphologic observations support a close link between Rac1 and endosomal processing of AAV2 that are consistent with Nox activation in newly formed vesicles containing AAV2.

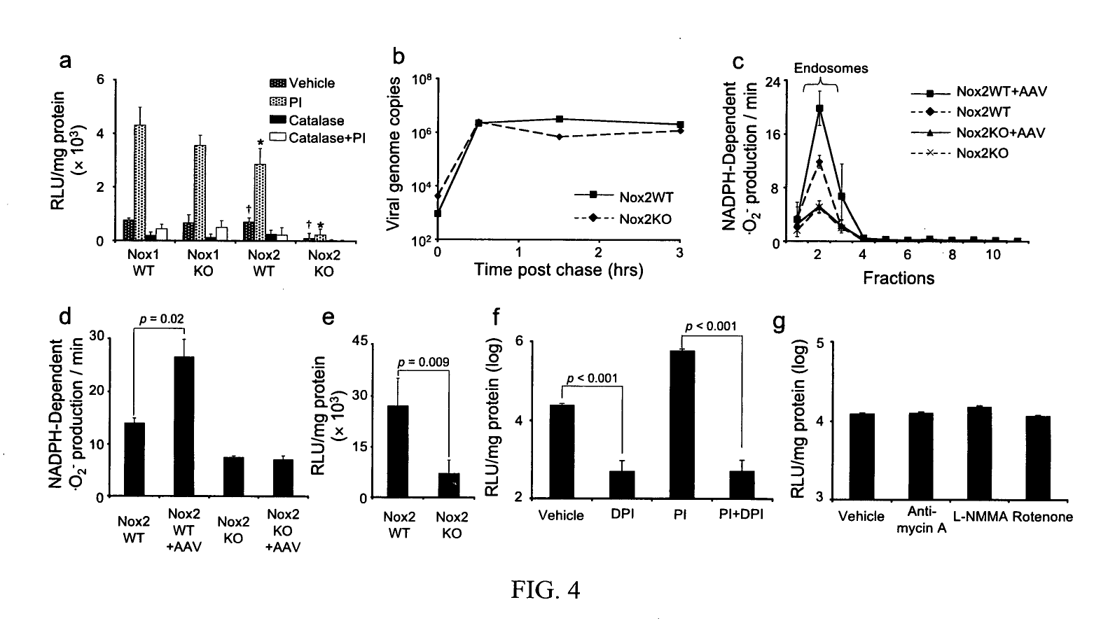

Because Rac1 is a known activator of Nox1 and Nox2, it was hypothesized that ROS generated following AAV2 infection were the result of either Nox1 and/or Nox2 activation in the endosomal compartment. To address this hypothesis, it was evaluated whether AAV2 transduction of Nox1^{-/-} and Nox2^{-/-} knockout primary mouse dermal fibroblasts (PMDF) was reduced in comparison to wild type littermate control PMDFs. Results from these studies (FIG. 4A) demonstrated that the presence of Nox1 did not significantly influence AAV2 transduction of PMDFs; baseline and proteasome inhibitor induced AAV2 transduction was similar between Nox1^{+/+} and Nox1^{-/-} PMDFs and catalase endosomal loading also inhibited transduction in all of these conditions. In contrast, AAV2 transduction in the absence and presence of proteasome inhibitors was significantly (p<0.001) reduced in Nox2^{-/-} PMDFs as compared to Nox2^{+/+} littermate control cells (FIG. 4A). This decrease was not due to impaired viral uptake in knockout cells, as no difference in the uptake of viral genome copies was observed between the Nox2^{+/+} and Nox2^{-/-} PMDFs (FIG. 4B). In contrast to Nox2+/+PMDFs, endosomal catalase loading did not significantly alter AAV2 transduction in Nox2^{-/-} PMDFs (FIG. 4A), suggesting that the lack of Nox2 was sufficient to clear the majority of endosomal H₂O₂ required to facilitate endosomal processing of AAV2. Nox2^{-/-} PMDFs also failed to induce NADPH-dependent .O₂⁻ production in the endosomal fraction following AAV2 infection, while a 2-fold induction was seen Nox2^{+/+} PMDFs (FIGS. 4C-D).

To further substantiate the importance of Nox2 in AAV2 transduction, additional in vivo experiments were performed with recombinant AAV2 delivery to the lung. As seen in primary PMDFs, AAV2 transduction of the lung following nasal delivery of virus demonstrated an about 5-fold lower level of luciferase transgene expression in Nox2^{-/-} mice, as compared to Nox2^{+/+} littermates (FIG. 4E). These findings strongly suggest that Nox2 is the primary source of endosomal ROS production in response to AAV2 infection and that Nox2-derived ROS are functionally important for AAV2 transduction. To confirm that NADPH oxidase was also required for transduction in transformed cells, AAV2 infection of HeLa cells was performed in the presence or absence of DPI (a known inhibitor of NADPH oxidases). Findings from these experiments clearly demonstrated that DPI effectively inhibited AAV2 transduction in the absence (100-fold) and presence (1000-fold) of proteasome inhibitors (FIG. 4F). In contrast, two inhibitors of mitochondria respiration (antimycin A and rotenone) or nitric oxide synthase (N-monomethyl-L-arginine acetate, L-NMMA) did not significantly affect AAV2 transduction (FIG. 3G), suggesting that these pathways for ROS production were not involved.

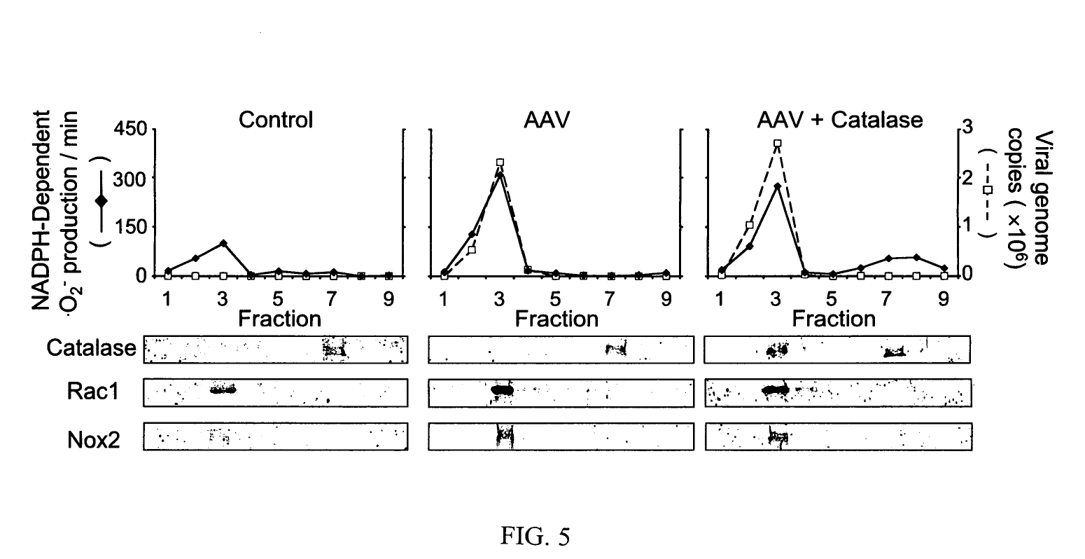

Studies thus far suggested that Rac1 and Nox2 are recruited to AAV2 containing endosomes following infection to facilitate redox-dependent transduction, and that catalase loading of AAV2-containing endosomes inhibited this process. Hence, Rac1, Nox2, and endosomally-loaded bovine catalase should all fractionated to the endosomal compartment with AAV2 virus following infection. To confirm this, each of these components was localized by subcellular fractionation of HeLa cells following infection in the presence and absence of exogenously supplemented bovine catalase in the media. Results from these studies demonstrated that indeed Rac1, Nox2, bovine catalase, and viral genomes all separated to the endosomal fractions coincident with peak NADPH oxidase activity induced by viral infection (FIG. 5). In contrast, endogenous cellular catalase separated in a denser fraction (#7), consistent with the higher density of peroxisomes where catalase is found. These studies also clearly demonstrated that catalase loading did not affect Nox activity or viral accumulation in the endosomal compartment at this early time point (20 minutes) following infection. Furthermore, following AAV2 infection a notable increase in both Nox2 and Rac1 in the endosomal fractions was seen, supporting the fact that these factors are recruited to AAV2 containing endosomes following infection.

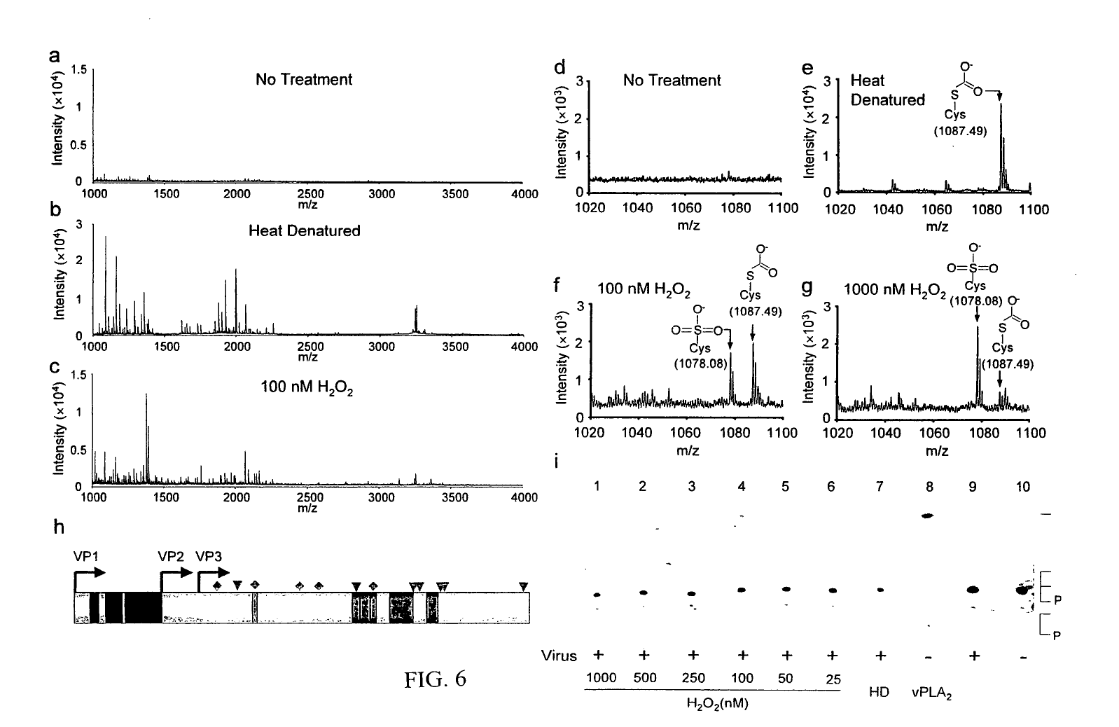

The capsid of AAV2 is composed of three proteins, VP1, VP2, and VP3, which differ in their N-terminal region (FIG. 6H). A detailed understanding of the functionally critical processing events that occur on the AAV capsid following infection remain unclear. Given the functional importance of endosomal ROS on AAV2 transduction seen in these studies, it was hypothesized that endosomal ROS was important for processing AAV2 virions.

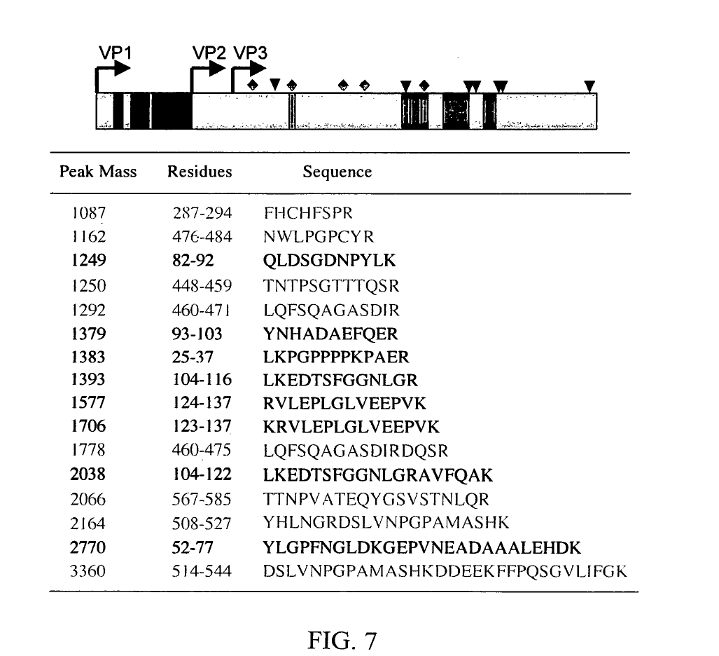

Since no gross morphologic changes in the AAV2 virion could be observed following treatment with 50 to 1000 nM H₂O₂ using electron microscopy (data not shown), the redox-mediated AAV2 capsid changes were likely very subtle structural modifications. To this end, a MALDI-TOF MS trypsin-sensitivity assay was developed to analyze minor structural change of AAV2 virions induced by H₂O₂ treatment. Intact AAV2 virions were extremely resistant to trypsin digestion and gave rise to no appreciable tryptic fragments by MS (FIG. 6A). In contrast, heat-treatment of purified AAV2 virions at 70° C. for 5 minutes allowed for a complete tryptic digestion and the subsequent identification of a majority of VP tryptic peptide fragments covering >90% of the VP amino acid sequence (FIG. 6B). Strikingly, pre-treatment of AAV2 virions with 100 nM of H₂O₂ resulted in a significantly enhanced tryptic digestion liberating a subset of peptides seen in the heat denatured virus (FIG. 6C). The corresponding position of H₂O₂-liberated tryptic peptide fragments as they correspond to the primary sequence of viral capsid proteins is shown in FIG. 6H and FIG. 7. Interestingly, these peptides are concentrated in several major regions, one in the unique N-terminus of VP1 associated with PLA₂ activity (FIG. 6H) and two adjacent to amino acid residues with proposed high surface accessibility in the virion (Xie et al., 2002).

These findings suggest several important implications for endosomal H₂O₂ function in the intracellular processing of AAV2.

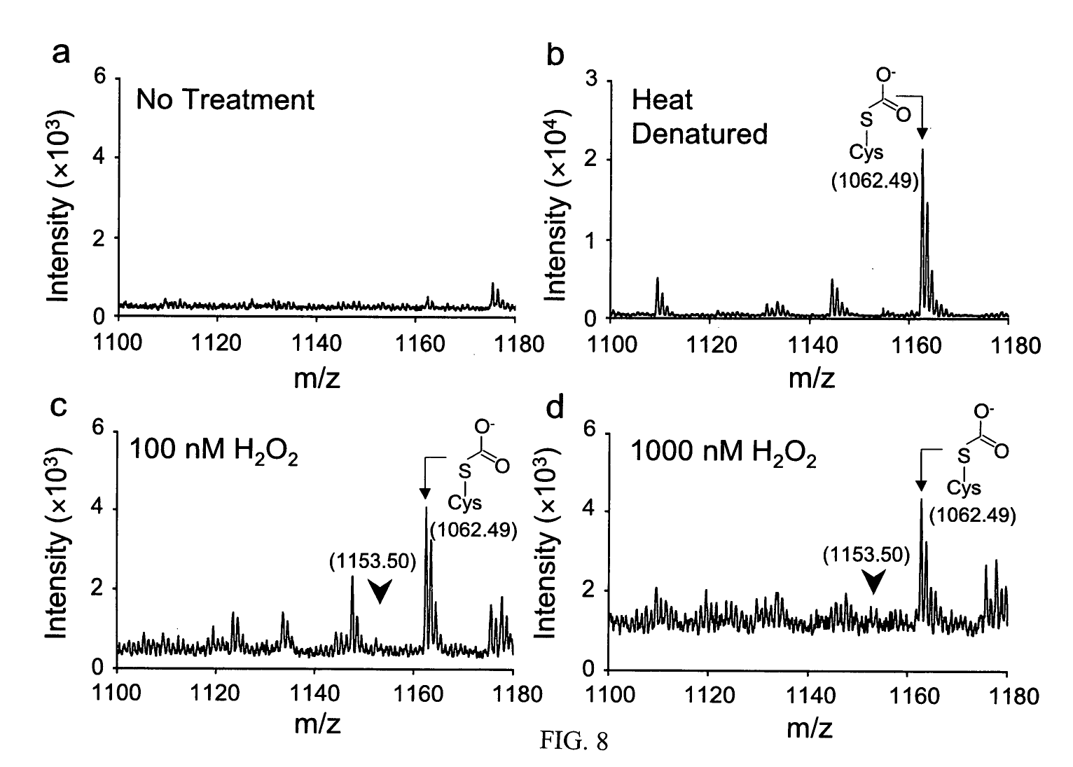

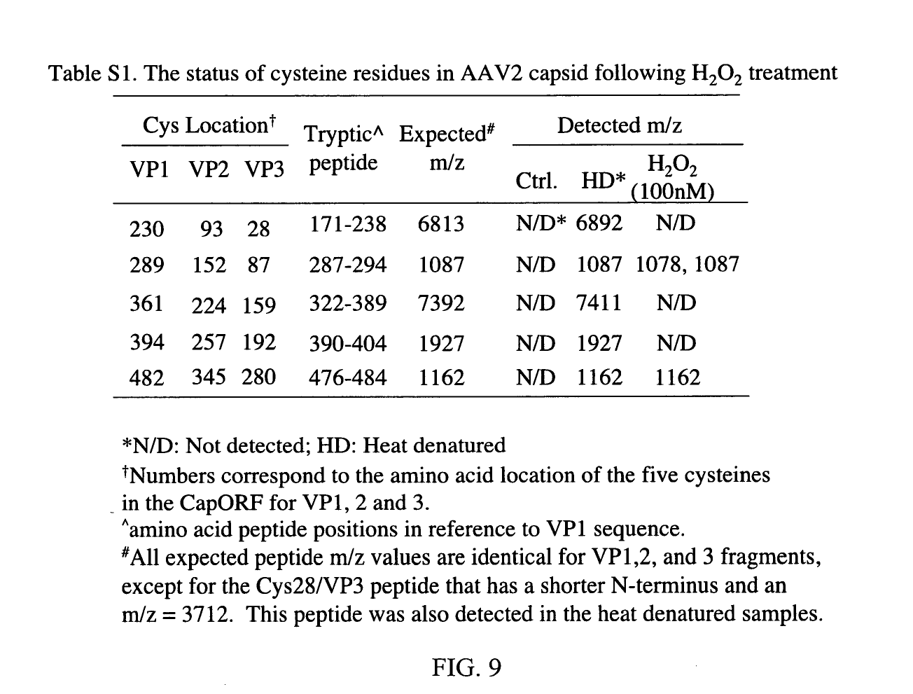

To dissect the molecular modifications induced by H₂O₂ that are responsible for mediating structural alterations in the AAV2 virions, it was hypothesized that redox-modification of cysteine residues on the viral capsid might facilitate this mechanism. Depending on the number of electrons transferred, redox modification of thiol groups can result in various products including disulfide bonds, sulfenic acid, sulfinic acid, sulfonic acid in addition to others (Paget et al., 2003). Using iodoacetamide cysteine modification and trypsin digestion, the status of the five cysteines in the AAV2 cap ORF (FIG. 6H) were analyzed in intact, heat-denatured, or H₂O₂-treated virions by MALDI-TOF MS (FIGS. 6, 8 and 9). Results demonstrated that all cysteines were modified by iodoacetamide in the heat-denatured virions (FIGS. 6B and E, FIG. 8, and FIG. 9) and no labeling of cysteines was observed with intact virions (FIGS. 6A and D, FIG. 8 and FIG. 9). In contrast, treatment of virions with 100 nM H₂O₂ led to a sulfonic (R—SO₃⁻) modification on the thiol group of a single cysteine common to VP1 (C289), VP2 (C152) and VP3 (C87) (FIG. 6F). In addition, treatment of virions with 1000 nM H₂O₂ increased the sulfonic modification of this specific cysteine, while simultaneously decreasing the iodo acetamide modification (FIG. 6G).

In contrast, C482 (referenced to VP1 sequence) within H₂O₂-treated virions demonstrated only the iodoacetamide modification (FIGS. 8-9). The corresponding peptides containing the remaining cysteines in the capsid were not detected following H₂O₂-treatment of virions (FIG. 9), suggesting that these regions were not exposed following H₂O₂ treatment for efficient trypsin digestion. In contrast, the sulfonic acid forming cysteine (C289) and non-redox modified cysteine (C482) were located in regions of the capsid accessible to tryptic digestion following treatment with 100 nM H₂O₂, resulting in the peptides FHCHFSPR and NWLPGPCYR, respectively (FIG. 6H and FIG. 7). The regions of VP2 containing C289 and C482 have also been proposed to be adjacent to the surface accessible amino acids in the capsid (Xie et al., 2002) (FIG. 6H).

It has been reported that an N-terminal region in the VP1 capsid protein contains phospholipase A₂ (PLA₂)-like motif and activity to cleave lipid chains that is highly conserved among most parvoviruses (Girod et al., 2002; Zadori et al., 2001). Mutations in the PLA₂ motif found in AAV2 significantly impairs replication of wt AAV2 at a step following viral entry (Girod et al., 2002; Zadori et al., 2001). Based on the fact that PLA₂ mutant AAV2 or porcine parvovirus enter cells effectively and traffic to the perinuclear late endosomal/lysosomal region efficiently, but fail to initiate viral DNA replication, PLA₂ activity of VP1 may be critical for endosomal processing of virus to the nucleus. Electron cryo-microscopy of AAV2 capsids revealed that the N-termini of VP1 is buried inside the intact virion, and partial denaturation is required to expose this region and PLA₂ activity (Girod et al., 2002; Kronenberg et al., 2005; Kronen et al., 2001; Zadori et al., 2001). The mechanism of in vivo activating AAV2 PLA₂ is not clear, though endosomal acidification has been reported to facilitate the exposure of N-VP1 of the minute virus of mice, a member of the parvovirus family (Mani et al., 2006).

It was hypothesized that the redox-directed exposure of the VP1 N-terminus by AAV2 infection might be important in directing conformational changes in the virion that liberate PLA₂ activity of VP1. To test this hypothesis, it was investigated whether H₂O₂ treatment could induce viral PLA₂ activity in purified recombinant AAV2 virions. Virus was treated with increasing concentrations of H₂O₂ (25 to 1,000 nM) at a H₂O₂:virion ratio ranging from 30:1 to 1, 200:1. Treated virions were assessed for PLA₂ activity by incubation with L-3-phosphatidylcholine (1,2-di[1-¹⁴C]oleoyl), a substrate for PLA₂. Interestingly, PLA₂ activity was mobilized from AAV2 virion at concentrations of H₂O₂ ranging from 50 to 500 nM (FIG. 6A). At the optimal concentration of 100 nM of H₂O₂, which was also the concentration that maximally induced trypsin-sensitivity of the virion, PLA₂ activity was greater than that seen following partial virion heat denaturation that had been previously used to evaluate such activity (FIG. 6I, compare lane 4 to lane 8). These results demonstrated that a relatively narrow window of H₂O₂ concentrations could activate PLA₂ activity in AAV2 virions.

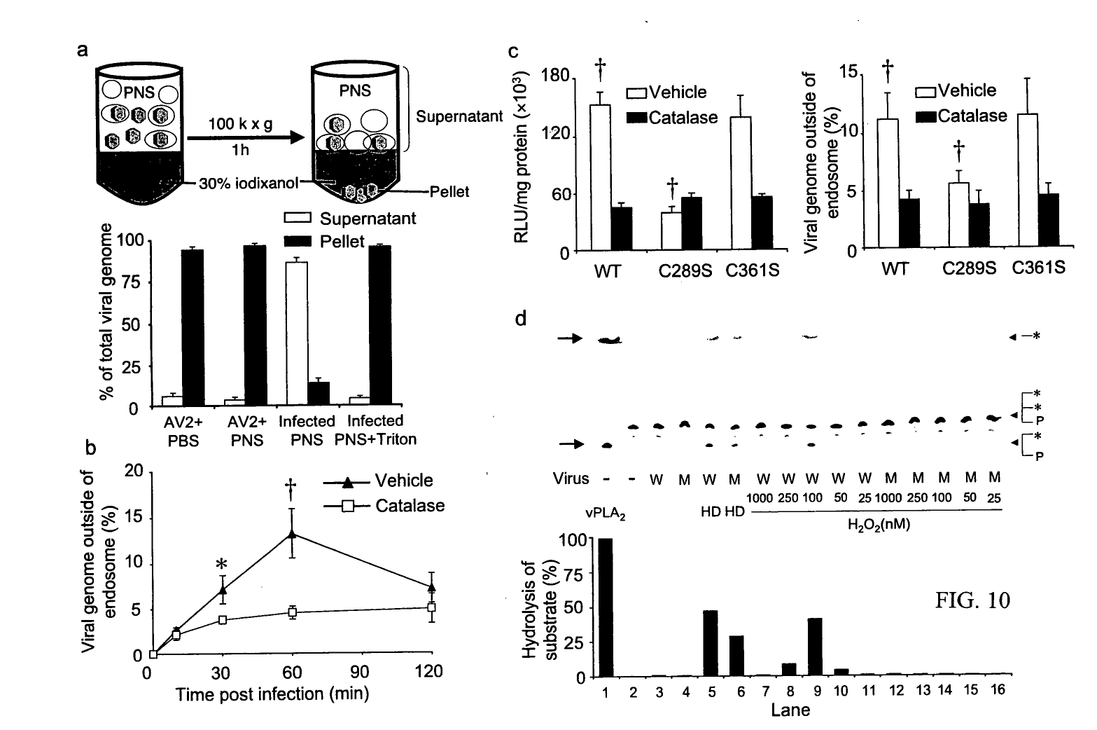

The studies thus far indicated that H₂O₂ treatment promotes conformational changes of AAV2 capsid that induce PLA₂ activity. It was hypothesized that the redox-dependent activation of capsid PLA₂ was important for endosomal escape of virions. To test this hypothesis, an iodixanol cushion to separate free cytoplasmic virions from those inside endosomes (FIG. 10A, top panel). Using reconstitution experiments, it was demonstrated that purified virions spiked into PBS or HeLa cells post-nuclear supernatants (PNS) predominantly pelleted through 30% iodixanol following high-speed centrifugation (100,000×g) (FIG. 10A, bottom panel). In contrast, the majority of virions (>85%) in PNS from HeLa cells infected with AAV2 for 1 hour remained in the supernatant, while the addition of 0.1% Triton X-100 to the PNS prior to fractionation liberated >95% of the virions into pellet (FIG. 10A, bottom panel). To study the importance of H₂O₂ in endosomal escape of AAV2, this system was utilized in combination with catalase endosomal loading. Results demonstrated that viral escape from endosomes (i.e., % in the pellet) peaked (about 15%) at 1 hour following infection in the absence of catalase, while catalase loading significantly inhibited viral escape about 3-fold (FIG. 8B). The reduction of free virus in the cytoplasmic fraction at 2 hours post-infection in the control (no catalase) samples likely represents rapid nuclear transport of free cytoplasmic virions. These results are consistent with the inhibitory effect of catalase on AAV2 transduction and support the hypothesis that H₂O₂-facilitated processing of virions is important for viral endosomal escape.

In vitro studies implicate sulfonic acid modification of Cys289 (referenced to VP1 sequence) in the redox events controlling H₂O₂-mediated AAV2 transduction. To better understand how this cysteine affects AAV2 infection in vivo, recombinant AAV2 virus with a C289S capsid mutation was prepared and tested for its redox-sensitivity of transduction in HeLa cells. Indeed, AAV2-C298S virus demonstrated significantly reduced transduction efficiency and compromised endosomal escape as compared to recombinant virus containing the wt capsid (FIG. 10C). Importantly, residual transduction with AAV2-C298S virus was not sensitive to catalase loading (FIG. 10C), suggesting that infection with this mutant virus was no longer redox-sensitive. As a control, we also mutated a cysteine (C361) that was not exposed following H₂O₂ treatment of virions. Analysis of transduction with a recombinant AAV2-C361S mutant luciferase virus demonstrated that transgene expression was not significantly different than that of virus with a wild type capsid (FIG. 10C). Neither the C298S capsid mutation or catalase loading altered the efficiency of viral entry as reflected by the total amount of viral genome in the PNS following infection (data not shown). In support of C298 being important for redox-dependent processing of the capsid, in vitro H₂O₂ treatment of purified AAV2-C298S virions failed to induce the PLA₂ activity in comparison to virions containing the wt capsid (FIG. 10D, compare lanes 12-16 to lanes 7-11). Interestingly, the C298S mutation did not functionally destroy the PLA₂ domain, as heat denatured C289S virions displayed PLA₂ activity at a level comparable to wild type virions (FIG. 10D, compare lane 6 to lane 5). These findings demonstrate that C298 is important for the redox-activation of the capsid PLA₂ domain and that this process of activation promotes endosomal escape of AAV2.

Structural analysis of AAV2 capsids using electron cryo-microscopy has revealed that the of PLA₂ motif-containing VP1 N-termini is organized as a globular structure buried inside the intact virion (Girod et al., 2002). Partial denaturation is required for the exposure of this region through the fivefold axes-channels on the capsid (Girod et al., 2002) and the in vitro activation of viral capsid PLA₂ (Kronenberg et al., 2005). Although PLA₂ activation by AAV2 capsids has been proposed to be essential for viral processing required for nuclear entry (Kronenberg et al., 2005), the mechanism of in vivo activation of AAV2 PLA₂ is unclear. These studies have discovered a mechanism whereby endosomal NADPH oxidase facilitates productive infection by AAV2 through the oxidation of its capsid. In this context, extremely low levels of H₂O₂ can act to structurally alter the virion by forming sulfonic acid on a unique cysteine(s) within the capsid. This redox-mediated event in turn liberates PLA₂ activity from the virion important for facilitating endosomal escape. Since the capsid is composed of approximately 60 VP protein subunits (all of which contain C289), it is presently unclear how many cysteines must be oxidized to liberate PLA₂ activity. However, quantitative cysteine labeling of virions following 100 nM H₂O₂ treatment suggests that the number of cysteines that form sulfonic acid in the capsid may be quite low (<5%, data not shown). Further elucidation of the redox-dependent structural alterations to viral capsids may lead to improvements in parvoviruses for gene therapy. Moreover, expanding these studies to pathogenic parvoviruses that also contain PLA₂ motifs, such as B19, may also aid in the development of anti-oxidants as anti-viral agents.

### SUMMARY

Viruses have evolved to effectively infect host cells by either inactivating cellular innate immune mechanisms or adapting to such mechanisms to the benefit of virus survival. Reactive oxygen species (ROS) derived from the phagocytic NADPH oxidase (Nox2^{gp91phox}) are one example of an innate immune response typically association with pathogen destruction. As described herein, infection with AAV2 stimulates Nox2-dependent endosomal ROS production and utilized the resultant H₂O₂ to facilitate productive endosomal processing of the virion. MADLI-TOF MS analysis demonstrated that nM quantities of H₂O₂ promoted exposure of the VP1 N-terminus capsid proteins within the virion leading to activation of a phospholipase A₂ motif shown to be critical for parvovirus infection. Those findings demonstrate a new mechanism by which a virus can utilizes host-pathways to productively process its capsid in the endosome, and provide insights into viral-host interaction.

Further elucidation on the interaction between virus and cellular redox balance improves the understanding of the life cycle of different viruses, but also helps to identify drug targets inhibiting replication of pathogenic viruses or promoting transduction with recombinant viruses used in gene therapy approaches.

### EXAMPLE II

### Methods

### Subcellular Fractionation

Buoyant density centrifugation was used for subcellular fractionation and isolation of endosomes containing Nox2 activity. Cells were washed twice with ice-cold PBS and scrapped into a 1.5 mL microfuge tube using the same buffer. The cells were pelleted and resuspended in homogenization buffer (HMB) containing 0.25 M sucrose, 20 mM HEPES pH 7.4, 1 mM EDTA, and an EDTA-free protease inhibitor cocktail. The cells were homogenized using nitrogen cavitation in a cell disruption high-pressure chamber (Parr instruments, Moline, Ill.). The pressure was raised to 650-psi for 5 minutes and released suddenly. The homogenate was centrifuged at 3000×g for 15 minutes to pellet unbroken cells, nuclei, and heavy mitochondria. The heavy mitochondrial supernatant (HMS) was bottom loaded into an iodixanol discontinuous gradient in a 12.5 mL SW41Ti ultracentrifuge tube using a previously described method with modifications (Graham et al., 1994; Xia et al., 1998).

The discontinuous gradient was composed of 1.25 mL HMB without EDTA followed by bottom loading of the following % iodixanol steps sequentially with 1.0 mL 2.5%, 1.0 mL 5%, 1.5 mL 9%, 1.5 mL 14%, 2.5 mL 19%, 1.5 mL 26%, and finally the HMS in 2 mL 32%. Iodixanol concentrations were prepared fresh using a 50% iodixanol working solution (WS) diluted with HMB without EDTA. The WS was prepared by adding 1 part buffer containing 0.25 M sucrose, and 120 mM HEPES pH 7.4 to 5 parts iodixanol 60% stock solution. The gradients were centrifuged at 100,000×g using an SW41Ti swinging rotor overnight at 4° C. The fractions were collected from the top of the tube using a fraction collector (Labconco, Kansas city, Mo.) in 500 μl fractions on ice. The density gradient was designed to optimally separate the following compartments based on previous studies (Billington et al., 1998; Graham et al., 1994; Graham, 2002; Graham et al., 1996; Plonne et al., 1999): Fr#1-5 plasma membrane (density 1.03-1.05 g/mL); Fr#7-13 endosomal compartment (density 1.055-1.11 g/mL); Fr#8-10 Golgi apparatus (density 1.06-1.09 g/mL); Fr#10-13 light endoplasmic reticulum (density 1.09-1.11 g/mL); Fr#13-18 lysozomes (density 1.11-1.13 g/mL): Fr#18-21 light mitochondria (density 1.13-1.15 g/mL); Fr#19-20 heavy endoplasmic reticulum (density 1.145 g/mL); Fr#21-24 peroxisomes (density 1.18-1.2 g/mL); and Fr#22-24 cytosolic proteins (density 1.26 g/mL).

### Vesicular Immuno-Isolation of Rac1 Redox-Active Endosomes

Affinity isolation of HA-tagged Rac1 endosomes was performed using methods previously described for immunoabsorption of Rab5 endosomes (Li et al., 2005; Trischler et al., 1999). Cells were infected with a recombinant adenovirus expressing HA-tagged Rac1 (Ad.HA-Rac1) 48 hours prior to IL-1β treatment at 1 ng/mL. Following iodixanol isolation of intracellular vesicles, one half of the combined peak vesicular fraction was used directly for biochemical analyses of superoxide production and the other half was used for immuno-affinity isolation using Dynabeads M-500 (Dynal Bioscience) coated with the anti-HA antibody. Prior to use, beads were coated with antibodies as follows: the secondary antibody (anti-rat) was conjugated to Dynabeads (4×10⁸ beads/mL) in 0.1 M of borate buffer (pH 9.5) for 24 hours at 25° C. with slow rocking. The beads were then placed into the magnet for 3 minutes and washed in 0.1% (w/v) BSA/PBS for 5 minutes at 4° C. A final wash in 0.2 M Tris (pH 8.5)/BSA was performed for 24 hours. Finally, the beads were resuspended in BSA/PBS and conjugated to 4 μg of primary anti-HA antibody per 10⁷ beads overnight at 4° C. and then washed in BSA/PBS. Vesicular fractions were mixed with 700 μL of coated beads in PBS containing 2 mM EDTA, 5% BSA, and protease inhibitors. The mixture was incubated for 6 hours at 4° C. with slow rocking, followed by magnetic capture and washing in the same tube three times (15 minutes each). Beads with HA-enriched endosomes were then resuspended in PBS. The bound pellets (P) and wash supernatants (S) were then evaluated for NADPH-dependent superoxide production and association of HA-Rac1, p67phox, SOD1, IL-IR1, TRAF6, TNFR1, TRAF2, and Rab5 by Western blotting.

### Results

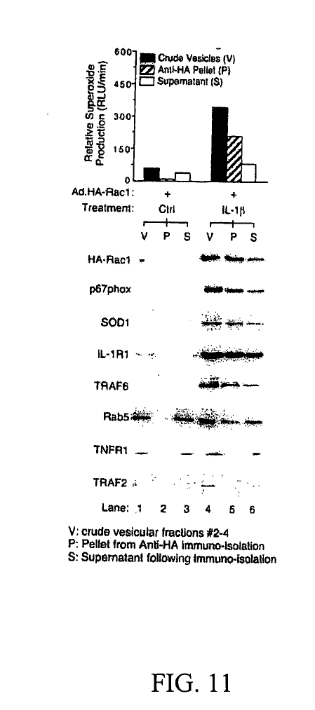

HA-Rac1 incorporation into crude vesicular fractions was significantly enhanced by IL-1β stimulation (FIG. 11, lane 4). Rac1 was found only at low levels in unstimulated vesicles (lane 1). These findings support the notion that Rac1 (an essential activator of Nox2) is specifically recruited to the endosomal compartment following IL-1β stimulation. Immuno-affinity isolation of HA-Rac1-bound endosomes demonstrated that the purification procedure was capable of isolating approximately 75% of the HA-immunoreactive endosomes (lane 5 versus lane 6). This was a similar efficiency as that previous reported for HA-Rab5 isolation from this cell line (Li et al., 2005). As anticipated, this fractional enrichment for HA-Rac1 in the anti-HA-bound pellet mirrored the enrichment seen in its capacity to produce NADPH-dependent .O₂. Similarly, SOD1, p67phox (a Nox2 activator subunit), IL-1R1, and the IL-1R1 specific effector TRAF6 were all enriched on HA-Rac1 endosomes relative to a general endosomal marker (Rab5). In the absence of IL-1β stimulation, SOD1 and p67phox failed to recruit to endosomal membranes and only low levels of IL-1R1/TRAF6 in the endosomal compartment was seen (lane 1). As a negative control for signal specificity, TNFR1 and its specific effector TRAF2 were also evaluated. No TNFR1/TRAF2 was recruited to IL-1β-activated, Rac1-containing endosomes (Lane 5). These findings provide direct evidence for the enrichment of SOD1 in redox-active endosomes containing ligand activated IL-1R1/TRAF6 complexes and Rac1.

Thus, affinity isolation of HA-tagged Rac1 endosomes following viral infection may be useful to identify new receptors important for AAV or other parvovirus receptor entry pathways, and is applicable to any type of virus that moves through Nox-active endosomes.

### REFERENCES

Albrich et al., FEBS Lett., 144:157 (1982).
Aniansson et al., Acta Pathol. Microbiol. Immunol. Scand. Sect. C, 92:357 (1984).
Babior et al., J. Clin. Invest., 58:989 (1976).
Babior et al., J. Lab. Clin. Med., 85:235 (1975).
Babior, N. Engl. J. Med., 298:659 (1978).
Baruchel et al., J. Leukoc. Biol., 52:111 (1992).
Basu et al., BMC Cell Biol., 2:15 (2001).
Bielski et al., J. Phys. Chem. Ref. Data, 14:1041 (1985).
Billington et al., Anal. Biochem., 258:251 (1998).
Borregaard et al., Blood, 89:3503 (1997).
Buffinton et al., Free Radic. Res. Commun., 16:99 (1992).
Cai et al., Free Radic. Biol. Med., 34:928 (2003).
Carter, Hum. Gene Ther., 16:541 (2005).
Choi et al., Nature, 435:347 (2005).
Clark et al., J. Clin. Invest., 85:714 (1990).
Clark, J. Infect. Dis., 161:1140 (1990).
Dahlgren et al., Infect. Immun., 47:326 (1985).
DeLeo et al., J. Leukoc. Biol., 60:677 (1996).
DeLeo et al., Proc. Natl. Acad. Sci. USA, 92:7110 (1995).
Dinauer et al., Nature, 327:717 (1987).
Dinauer, et al., Crit. Rev. Clin. Lab. Sci., 30:329 (1993).
Ding et al., Mol. Ther., (2006)
Dollard et al., J. Virol., 68:7800 (1994).
Dorseuil et al., J. Leukocyte Biol., 58:108 (1995).
Droge et al., Immunol Today, 13:211 (1992).
Duan et al., J. Clin. Invest., 105:1573 (2000).
Enyedi et al., Cell, 70:879 (1992).
Fantone, et al., Hum. Pathol., 16:973 (1985).
Fearon et al., Science, 272:50 (1996).
Finan et al., J. Biol. Chem., 269:13752 (1994).
Gabig et al., Blood, 85:804 (1995).
Girod et al., J. Gen. Virol., 83:973 (2002).
Graham et al., Anal. Biochem., 220:367 (1994).
Graham et al., Z. Gastroenterol., 34:76 (1996).
Graham, Scientific World Journal, 2:1400 (2002).
Halliwell, B. and Gutteridge, J. M. C. Free radicals in biology and medicine, Third Edition; Oxford Science Publications, pp 33-34 (1998).
Hansen et al., J. Virol., 74:992 (2000).
Harrison et al., J. Biol. Chem., 251:1371 (1976).
Hauck et al., J. Virol., 78:13678 (2004).
Heyworth et al., J. Biol. Chem., 269:30749 (1994).
Heyworth et al., J. Clin. Invest., 87:352 (1991).
Hoffmann et al., Science, 284:1313 (1999).
Hyslop et al., Free Radic. Biol. Med., 19:31 (1995).
Iyer et al., J. Biol. Chem., 269:22405 (1994).
Jackson et al., Hematol. Oncol. Clin. North. Am., 2:317 (1988).
Janeway et al., Cell, 76:275 (1994).
Kaludov et al., Hum. Gene Ther., 13:1235 (2002).
Kamata et al., Cell, 120:649 (2005).
Kannan et al., Free Radic. Biol. Med., 37:229 (2004).
Kettle et al., Redox Rep., 3:3 (1997).
Klebanoff, Proc. Assoc. Am. Physicians, 111:383 (1999).
Kleinberg et al., J. Biol. Chem., 265:15577 (1990).
Kronenberg et al., EMBO Rep., 2:997 (2001).
Kronenberg et al., J. Virol., 79:5296 (2005).
Lambeth, Nat. Rev. Immunol., 4:181 (2004).
Leto et al., Proc. Natl. Acad. Sci. U.S.A., 91:10650 (1994).
Leusen et al., J. Clin. Invest., 93:2120 (1994).
Leusen et al., J. Exp. Med., 180:2329 (1994).
Li et al., J. Biol. Chem., 281:1495 (2006a).
Li et al., Mol. Cell. Biol., 26:140 (2006b).
Liochev et al., Arch. Biochem. Biophys., 337:115 (1997).
Loguercio et al., Free Radic. Biol. Med., 34:1 (2003).
Lomax et al., Science, 245:409 (1989)
Lomax et al., Science, 246:987 (1989).
Mani et al., J. Virol., 80:1015 (2006).
McFadden, Science, 279:40 (1998).
McNally et al., J. Biolumin. Chemilumin., 11:99 (1996).
Mihm et al., Aids, 5:497 (1991).
Nakamura et al., Antioxid. Redox. Signal, 4:455 (2002).
Nakanishi et al., J. Biol. Chem., 267:19072 (1992).
Nauseef et al., Mandell, G. L., Bennett, J. E. and Dolin, R. (Eds.); Fifth Edition, Churchill Livingstone, Philadelphia, USA., Chapter 8: Granulocytic Phagocytes (2000).
Nauseef, Hematol. Oncol. Clin. North Am., 2:135 (1988).
Needleman and Wunsch, J. Mol. Biol., 48:443 (1970).
Newman et al., J. Exp. Med., 180:359 (1994).
Oda et al., Science, 244:974 (1989).
Ogle et al., J. Immunol. Methods, 115:17 (1988).
Paget et al., Annu. Rev. Genet., 37:91 (2003).
Park et al., J. Biol. Chem., 267:17327 (1992).
Park et al., Mol Cell Biol, 24:4384 (2004).
Pearson and Lipman, Proc. Natl. Acad. Sci. USA, 85:2444 (1988).
Plonne et al., Anal. Biochem., 276:88 (1999).
Quinn et al., J. Biol. Chem., 268:20983 (1993).
Quinn et al., Nature, 342:198 (1989).
Rhee et al., Sci. STKE, 2000:PE1 (2000).
Rosen et al., J. Exp. Med., 149:27 (1979).
Rotrosen et al., Science, 256:1459 (1992).
Russell et al., Proc. Natl. Acad. Sci. USA, 92:5719 (1995).
Sambrook, Fritsch, and Maniatis, Molecular Cloning: A Laboratory Manual, Second Edition (1989).
Sanlioglu et al., Gene Ther., 6:1427 (1999).
Sanlioglu et al., J. Virol., 74:9184 (2000).
Sanlioglu et al., Virus Res., 104:51 (2004).
Schettler et al., Eur. J. Biochem., 197:197 (1991).
Schnitzler et al., Adv. Exp. Med. Biol., 418:897 (1997).
Schreck et al., J. Virol., 66:6288 (1992).
Schwarz, Free Radic. Biol. Med., 21:641 (1996).
Segal et al., Biochem. J., 284:781 (1992).
Segal et al., Trends Biochem. Sci., 18:43 (1993).
Seisenberger et al., Science, 294:1029 (2001).
Shisler et al., Science, 279:102 (1998).
Smith and Waterman, Adv. Appl. Math., 12:482 (1981).
Smith et al., Blood, 77:673 (1991).
Sumimoto et al., Proc. Natl. Acad. Sci. USA, 91:5345 (1994).
Teoh et al., J. Virol., 79:5799 (2005).
Trischler et al., J. Cell Sci., 112:4773 (1999).
Van Dalen et al., Biochem. J., 327:487 (1997).
Vliet et al., J. Biol. Chem., 272:7617 (1997).
Volpp et al., Science, 242:1295 (1988).
Wagner et al., Hum. Gene Ther., 13:1349 (2002).
Weinbaum et al., Nature, 286:725 (1980).
Weiss, N. Engl. J. Med., 320:365 (1989).
Whitin et al., Blood, 66:1182 (1985).
Wientjes et al., Biochem. J., 296:557 (1993).
Williams et al., Proc. Natl. Acad. Sci. USA, 74:1204 (1977).
Xia et al., Biochemistry, 37:16465 (1998).
Xie et al., Proc. Natl. Acad. Sci. USA, 99:10405 (2002).
Yan et al., Meth. Enz., 346:334 (2002).
Yan et al., J. Virol., 78:2863 (2004).
Yan et al., J. Biol. Chem., 281:29684 (2006).
Young et al., J. Virol., 74:3953 (2000).
Zadori et al., Dev. Cell, 1:291 (2001).
Zipfel et al., Biochem. Biophys. Res. Commun., 232:209 (1997).

All publications, patents and patent applications are incorporated herein by reference. While in the foregoing specification, this invention has been described in relation to certain preferred embodiments thereof, and many details have been set forth for purposes of illustration, it will be apparent to those skilled in the art that the invention is susceptible to additional embodiments and that certain of the details herein may be varied considerably without departing from the basic principles of the invention.

What is claimed:

1. A method to identify an agent that alters parvovirus transduction of mammalian cells, comprising:
a) contacting mammalian cells, one or more agents and a redox sensitive parvovirus, to yield a mixture; and
b) identifying one or more of the agents in the mixture that alter endosomal NADPH oxidase activity relative to corresponding mammalian cells contacted with the parvovirus but not the one or more agents.

2. The method of claim 1 wherein parvovirus transduction is inhibited.

3. The method of claim 1 wherein parvovirus transduction is enhanced.

4. The method of claim 1 wherein the parvovirus is a pathogenic parvovirus.

5. The method of claim 1 wherein the parvovirus is adeno-associated virus (AAV).

6. The method of claim 1 wherein the parvovirus is recombinant AAV.

7. A method to identify viral capsid modifications that enhance parvovirus transduction of mammalian cells, comprising:
a) contacting mammalian cells and a parvovirus having a modified viral capsid, wherein at least one modification is an alteration in the number or position of redox sensitive residues in the capsid; and
b) identifying whether the transduction of the mammalian cells by the modified parvovirus is altered relative to transduction of corresponding mammalian cells by a corresponding unmodified parvovirus.

8. The method of claim 7 wherein transduction by the modified parvovirus is enhanced.

9. The method of claim 7 wherein the modification is an increase in the number of redox-sensitive residues.

10. The method of claim 7 wherein the modification is an increased number of cysteines, lysines, histidines, or methionines, or any combination thereof.

11. The method of claim 7 wherein the capsid is post translationally modified.

12. A method to enhance parvovirus infection of mammalian cells, comprising:
contacting mammalian cells with parvovirus and an agent that enhances endosomal NADPH oxidase activity.

13. The method of claim 12 wherein the parvovirus is adeno-associated virus (AAV).

14. The method of claim 13 wherein the AAV is recombinant AAV.

15. A method to enhance transgene expression in a mammalian cell, comprising contacting mammalian cells with an amount of an agent selected to enhance endosomal NADPH oxidase activity and an amount of a recombinant parvovirus having a transgene, so as to enhance expression of the transgene.

16. The method of claim 15 wherein the transgene encodes a therapeutic gene product.

17. The method of claim 16 wherein the gene product is a polypeptide or peptide.

18. The method of claim 15 wherein the cells are lung cells, epithelial cells, liver cells, muscle cells, hematopoietic cells, heart cells or neuronal cells.

19. The method of claim 15 wherein the cells are human cells.

20. The method of claim 15 wherein the cells are non-human mammalian cells.

21. A method to inhibit parvovirus infection of mammalian cells, comprising:
contacting mammalian cells with parvovirus and an agent that inhibits NADPH oxidase activity.

22. A method to alter parvovirus infection of mammalian cells, comprising:
contacting mammalian cells with a parvovirus the capsid of which is modified to alter the number or position of redox sensitive amino acid residues.

23. The method of claim 22 wherein the number of cysteine, lysine, histidine, or methionine residues, or any combination thereof, is altered.

24. A method to alter viral production, comprising:
contacting mammalian cells with a parvovirus the capsid of which is modified to alter the number or position of redox sensitive amino acid residues.

25. A method to identify an agent that alters NADPH oxidase activity in parvovirus transduced mammalian cells, comprising:
providing mammalian cells contacted with an agent and a parvovirus; and
identifying whether the agent alters NADPH oxidase activity in the parvovirus containing cells relative to mammalian cells contacted with the parvovirus but not contacted with the agent.

26. The method of claim 25 wherein the agent enhances NADPH oxidase activity.

27. The method of claim 25 wherein the agent decreases NADPH oxidase activity.

28. The method of claim 21 wherein the agent that inhibits NADPH oxidase activity is DPI, apocynin or a combination thereof.

29. The method of claim 28 wherein the parvovirus is adeno-associated virus 2 (AAV2).

30. A method to identify a viral receptor or co-receptor comprising:
isolating Rac containing endosomes from mammalian cells infected with a virus and identifying molecules in the virus infected Rac containing endosomes that are not present in Rac containing endosomes in uninfected mammalian cells.

31. The method of claim 30 wherein the virus is a parvovirus.

32. The method of claim 31 wherein the parvovirus is a adeno-associated virus (AAV).

33. The method of claim 30 wherein in the Rac containing endosomes comprise recombinant Rac.

34. The method of claim 33 wherein the recombinant Rac comprises a fusion protein.

35. The method of claim 30 wherein the virus is a pathogenic virus.

36. The method of claim 35 wherein the pathogenic virus is B19.

37. The method of claim 30 wherein the virus is an adenovirus, poxvirus, lentivirus, hepatitis virus, parvovirus, coxsackievirus or influenza virus.
